(19) United States
(12) Patent Application Publication
(10) Pub. No.: US 2026/0125698 A1
(43) Pub. Date: May 7, 2026

(54) INDUCTION OF MEIOTIC RECOMBINATION USING A CRISPR SYSTEM

(71) Applicants: MEIOGENIX, PARIS (FR); INSTITUT CURIE, PARIS CEDEX 05 (FR); CENTRE NATIONAL DE LA RECHERCHE SCIENTIFIQUE, PARIS (FR); SORBONNE UNIVERSITE, PARIS (FR)

(72) Inventors: ALAIN NICOLAS, PARIS (FR); ALEXANDRE SERERO, COLOMBES (FR); LUC MATHIS, BOURG-LA-REINE (FR)

(21) Appl. No.: 18/878,108

(22) PCT Filed: Jun. 23, 2023
(86) PCT No.: PCT/EP2023/067164

§ 371 Date: Dec. 23, 2024

(30) Foreign Application Priority Data
Jun. 24, 2022 (EP) 22305923.9

(51) Int. Cl. C12N 15/82; C12N 9/22; C12N 15/11

(52) U.S. Cl. CPC C12N 15/8274; C12N 9/226; C12N 15/11; C12N 15/8213; C12N 15/8238; C12N 2310/20; C12N 2830/002

(57) ABSTRACT

The present invention relates to methods for inducing targeted meiotic recombination in a eukaryotic cell, wherein the method is designed to maintain efficient induction of meiotic recombination using a CRISPR system even after a large number of mitotic cell cycles by reducing of the activity of CRISPR nuclease outside of meiosis and/or using several guide gRNAs specific of different sites in a targeted chromosomal region thereby increasing the probability to retain at least one intact target site for gRNA available in each meiotic cell.

### CROSS-REFERENCE TO RELATED APPLICATION

This application is the U.S. national stage application of International Patent Application No. PCT/EP2023/067164, filed Jun. 23, 2023.

### REFERENCE TO AN ELECTRONIC SEQUENCE LISTING

The contents of the electronic sequence listing (311252000600SUBSEQLIST.xml; Size: 160,729 bytes; and Date of Creation: Oct. 7, 2025) is herein incorporated by reference in its entirety.

### FIELD

The present invention pertains to the field of targeted genetic modifications in eukaryotes. It relates in particular to a process for modifying a eukaryotic cell by inducing targeted meiotic recombination.

### TECHNOLOGICAL BACKGROUND OF THE INVENTION

The modification of genetic material of eukaryotic organisms has developed greatly over the last twenty years, and has found applications in plants, humans, animal cells as well as micro-organisms such as yeasts, in particular in agriculture, human health, food and environmental protection.

Yeasts find their application in a wide variety of industrial fields. Because many species are harmless, yeasts are in particular used in the food industry as fermentation agent in baking, brewing, wine-making or distilling, or in the form of extracts as nutritional elements or flavouring agents. They can also find use in the industrial production of bioethanol or molecules of interest such as vitamins, antibiotics, vaccines, enzymes, or steroid hormones, or in processes for degrading cellulosic materials. Similarly, plants are used in many industrial fields, whether in the agri-food, cosmetic or pharmaceutical industries.

The diversity of industrial applications of yeast and plants implies that there is a constant demand for yeast strains and plant varieties with improved traits or, at least, adapted to a new use or new culture conditions. In order to obtain a eukaryotic cell or organism with a particular trait, the person skilled in the art can use sexual reproduction and select a hybrid cell or organism providing the desired combination of parental traits. This method is however random and the selection step can lead to significant delays, in particular in the case of yeasts and plants.

Alternatively, the skilled person can also modify the genetic heritage of a cell or an organism by a recombinant DNA technique. This modification can nevertheless constitute an impediment to its exploitation, whether for regulatory, sanitary or environmental reasons, in particular in the case of plants considered as genetically modified organisms (GMO).

A third alternative consists of causing a reassortment of alleles of paternal and maternal origin in the genome, during meiotic recombination. Meiotic recombination is an exchange of DNA between homologous chromosomes during meiosis. After DNA replication, recombination is initiated by the formation of double-strand breaks in one (or the other) of the chromatids of the homologous chromosomes, followed by the repair of these breaks, using a chromatid of the homologous chromosome as a template. However, meiotic recombination has the disadvantage of being non-uniform. Indeed, the double-strand break sites at the origin of recombination are not distributed homogeneously in the genome. A distinction can thus be made between so-called ‘hot’ chromosomal regions where the frequency of recombination is high, and so-called ‘cold’ chromosomal regions where the frequency of recombination can be up to 100 times lower.

The control of double-strand break formation and, hence, of meiotic recombination, is crucial for the development of genetic engineering techniques. In particular, it has been shown that it is possible to modify the double-strand break formation sites by fusing Spo11 with the DNA-binding domain of the transcriptional activator Gal4 (Pecina et al., 2002, Cell, 111, 173-184 or WO/2004/016795) or with a CRISPR nuclease (see e.g. International patent applications WO 2016/120480, WO 2021/234315 and WO 2021/234317).

In contrast to the germline of animals, plants spend most of their life cycle maintaining a limited population of undifferentiated stem cells that are activated to become the germline only for a brief time in the plant's life. This process involves a switch in cell identity, which necessitates a distinct change in gene expression patterns. In particular, the repair of DNA double-strand breaks (DSBs) may occur via different mechanisms in somatic tissues. These DNA DSB are predominantly repaired via nonhomologous end-joining (NHEJ) during the growth of somatic tissues whereas they are predominantly repaired via homologous recombination (HR) during meiosis. NHEJ is an error-prone process that simply fuses the two broken ends together, often after some chewing back of the DNA, thus creating insertion/deletion events. On the other side, HR is essentially error-free as it uses the genetically identical sister chromatid as a template for repair.

The main concern for the application of the CRISPR technology in plant is to efficiently generate heritable mutations in T2 offspring population and to limit non-heritable mutations that can be induced in somatic cells. Indeed, during somatic growth, the repair of CRISPR DSB induces insertion/deletion, in the region targeted by the guide RNAs. The target sequences recognized by gRNAs may thus be destroyed by NHEJ mutagenesis with a certain probability at each cell cycle, usually between 5% and 60% per cycle. The result of this random mutagenesis is a chimerism of the transgenics that express CRISPR/Cas system throughout the cell/organism life cycle. The net result is a progressive decrease over time of the number of target sites available for cleavage by the gRNA/CRISPR/Cas system. In organisms that undergo a large number of mitotic cell cycles before meiosis such as plants, the availability of CRISPR targeted sites when cells enter flowering or sexual reproduction cycles thus becomes unpredictable, lowering the efficiency of the CRISPR system during meiosis.

Therefore, there is a need to develop methods that allow efficient induction of meiotic recombination using a CRISPR system in eukaryotic organisms, and in particular in organisms that undergo a large number of mitotic cell cycles before meiosis such as plants.

### SUMMARY OF THE INVENTION

The inventors herein provide different strategies to maintain efficient induction of meiotic recombination using a CRISPR system even after a large number of mitotic cell cycles. These strategies are essentially based on the reduction of the activity of CRISPR nuclease outside of meiosis, preferably outside of meiosis prophase, and/or on the use of several guide gRNAs specific of different sites in the targeted chromosomal region thereby increasing the probability to retain at least one intact target site for gRNA available in each meiotic cell.

In particular, the methods envisioned herein provide strategies for:
stimulating crossing-over during meiosis thanks to the activity of a CRISPR nuclease; and
reducing/inhibiting the activity of this nuclease outside of meiosis, in particular to reduce/inhibit nuclease activity in somatic cells.

Thus, in a first aspect, the present invention relates to a method for inducing targeted meiotic recombination in a non-human eukaryotic cell.

Such method comprises:
introducing into said cell:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
wherein the activity of the CRISPR nuclease is repressed during the mitotic phase, and wherein the CRISPR nuclease is not fused with a Spo11 protein.

Preferably, the present invention relates to a method for inducing targeted meiotic recombination in a cell, said method comprising:
introducing into said cell:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
wherein the activity of the CRISPR nuclease is repressed outside of meiosis,
wherein the CRISPR nuclease is not fused with a Spo11 protein, and
wherein the cell is a yeast cell, a plant cell or a fungus cell.

In a second aspect, the invention relates to a method for inducing targeted meiotic recombination in a non-human eukaryotic cell, said method comprising:
introducing into said cell:
a) a CRISPR nuclease, preferably a class II CRISPR nuclease or a nucleic acid encoding said nuclease,
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region; and
c) a fusion protein comprising a Spo11 protein operably linked to a DNA binding domain, or a nucleic acid encoding said fusion protein, preferably under the control of a meiosis-specific promoter; and
inducing said cell to enter meiotic prophase I,
wherein the CRISPR nuclease is not fused with a Spo11 protein and, optionally wherein the activity of the CRISPR nuclease is repressed during the mitotic phase.

Preferably, the invention concerns method for inducing targeted meiotic recombination in a cell, said method comprising:
introducing into said cell:
a) a CRISPR nuclease, preferably a class II CRISPR nuclease or a nucleic acid encoding said nuclease,
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region; and
c) a fusion protein comprising a Spo11 protein operably linked to a DNA binding domain, or a nucleic acid encoding said fusion protein, preferably under the control of a meiosis-specific promoter; and
inducing said cell to enter meiotic prophase I,
wherein the CRISPR nuclease is not fused with a Spo11 protein,
optionally wherein the activity of the CRISPR nuclease is repressed outside of meiosis, and wherein the cell is a yeast cell, a plant cell or a fungus cell.

Preferably, the activity of the CRISPR nuclease is repressed outside of meiosis.

In a third aspect, the invention further concerns a method for generating variants of a non-human eukaryotic organism, said method comprising:
introducing into a cell of said organism:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the activity of the CRISPR nuclease is repressed during the mitotic phase, and wherein the CRISPR nuclease is not fused with a Spo11 protein.

Preferably, the invention concerns a method for generating variants of an organism, said method comprising:
introducing into a cell of said organism:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the activity of the CRISPR nuclease is repressed outside of meiosis,
wherein the CRISPR nuclease is not fused with a Spo11 protein, and
wherein the organism is a yeast, a plant or a fungus.

In a fourth aspect, the invention concerns a method for generating variants of a non-human eukaryotic organism, said method comprising:
introducing into a cell of said organism:
a) a CRISPR nuclease, preferably a class II CRISPR nuclease or a nucleic acid encoding said nuclease, and
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the CRISPR nuclease is not fused with a Spo11 protein and, optionally wherein the activity of the CRISPR nuclease is repressed during the mitotic phase.

Preferably, the invention concerns method for generating variants of an organism, said method comprising:
introducing into a cell of said organism:
a) a CRISPR nuclease, preferably a class II CRISPR nuclease or a nucleic acid encoding said nuclease, and
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the CRISPR nuclease is not fused with a Spo11 protein and wherein the activity of the CRISPR nuclease is repressed outside of meiosis, and
wherein the organism is a yeast, a plant or a fungus.

The activity of the CRISPR nuclease may be repressed during the mitotic phase by placing the expression of said CRISPR nuclease and/or said guide RNAs under the control of a promoter inducible during the meiotic phase or a meiosis-specific promoter.

Preferably, the activity of the CRISPR nuclease is repressed outside of meiosis by placing the expression of said CRISPR nuclease and/or said guide RNAs under the control of a promoter inducible during the meiotic phase or under the control of a meiosis-specific promoter.

In particular, the meiosis specific promoter is selected from the group consisting of AtDMC1 promoter, Rec8 promoter, Spo13 promoter, MGE1p promoter, MGE2p promoter, MGE3p promoter, ZmDMC1 promoter, Maize Spo11-1 promoter (ZmSPO11-1), Tomato Spo11-1 promoter (SlSPO11-1) and Arabidopsis Spo11-1 promoter (AtSPO11-1), and variant thereof exhibiting at least 80%, 85%, 90%, 95%, 96%, 97%, 98%, or at least 99% identity to any of these promoters and a meiosis specific promoter activity, preferably from the group consisting of Maize Spo11-1 promoter (ZmSPO11-1, SEQ ID NO: 103) Tomato Spo11-1 promoter (SlSPO11-1, SEQ ID NO: 104) and Arabidopsis Spo11-1 promoter (AtSPO11-1, SEQ ID NO: 105), and variant thereof exhibiting at least 80%, 85%, 90%, 95%, 96%, 97%, 98%, or at least 99% identity to any of SEQ ID NO: 103, 104 or 105 and a meiosis specific promoter activity.

The activity of the CRISPR nuclease may be repressed during the mitotic phase by expressing an inhibitor of CRISPR endonuclease activity during the mitotic phase.

Preferably, the activity of the CRISPR nuclease is repressed outside of meiosis by expressing an inhibitor of CRISPR endonuclease activity outside of meiosis.

The activity of the CRISPR nuclease may also be repressed during the mitotic phase by using a system triggering the degradation of the CRISPR nuclease during the mitotic phase. In particular, the system triggering the degradation of the CRISPR nuclease during the mitotic phase is an auxin-inducible degron system.

Preferably, the activity of the CRISPR nuclease is repressed outside of meiosis by placing the expression of the CRISPR nuclease and/or the guide RNA(s) under the control of a promoter inducible during the meiotic phase or a meiosis-specific promoter and by expressing an inhibitor of CRISPR endonuclease activity outside of meiosis.

The activity of the CRISPR nuclease may be repressed during the mitotic phase by using CRISPR nuclease which is a variant of a wild-type CRISPR nuclease, said variant exhibiting a reduced endonuclease activity by comparison to said wild-type CRISPR nuclease.

Preferably, the activity of the CRISPR nuclease is repressed outside of meiosis by using a CRISPR nuclease which is a variant of a wild-type CRISPR nuclease, said variant exhibiting a reduced endonuclease activity by comparison to said wild-type CRISPR nuclease.

In the methods described herein, the cell may comprise a plurality of guide RNAs targeting the same chromosomal region, or a plurality of nucleic acids encoding said guide RNAs.

Preferably, the cell comprises at least two, at least three, at least four, or at least five, preferably at least three or five guide RNAs targeting the same chromosomal region.

In particular, the cell comprises at least five guide RNAs targeting the same chromosomal region or a plurality of nucleic acids encoding said guide RNAs, said chromosomal region being of less than 30 kilobase (kb) long.

Particularly, the cell comprises seven or more guide RNAs targeting the same chromosomal region or a plurality of nucleic acids encoding said guide RNAs, said chromosomal region being of less than 30 kilobase (kb) long.

Preferably, the cell comprises ten or more guide RNAs targeting the same chromosomal region or a plurality of nucleic acids encoding said guide RNAs, said chromosomal region being of less than 30 kilobase (kb) long.

In a particular embodiment, the cell further comprises a fusion protein comprising a Spo11 protein operably linked to a DNA binding domain, or a nucleic acid encoding said fusion protein, preferably under the control of a meiosis-specific promoter. Preferably, the DNA binding domain is selected from the group consisting of TAL effector DNA binding domains, B3 DNA binding domains, zinc finger DNA binding domains, helix-turn-helix DNA binding domains, leucine zipper DNA binding domains, HMG-box domains, transcription factor DNA binding domains such as GAL4 binding domain, inactivated CRISPR nucleases and inactivated meganucleases. Preferably, such fusion protein is placed under the control of a meiosis specific promoter.

The CRISPR nuclease used in the invention is preferably selected from the group consisting of Cas9 and Cpf1 nucleases.

Alternatively, the CRISPR nuclease used in the invention is a dead Cas9 (dCas9) or a dead Cpf1 (dCpf1).

The Spoil protein is Spo11-1, Spo11-2 or Spo11-3.

Preferably, the Spo11 protein is from Oryza sativa, Brassica campestris, Zea mays, Capsicum baccatum, Carica papaya or Solanum lycopersicum.

Preferably, the cell does not comprise a fusion protein comprising a CRISPR nuclease and a Spo11 partner, in particular a Spo11 partner selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto.

Particularly, the cell does not comprise a fusion protein comprising a CRISPR nuclease and a Spo11 partner, in particular a Spo11 partner selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto.

In particular, the cell does not comprise a fusion protein comprising i) a CRISPR nuclease and ii) a DNA binding domain, or a protein/amino acid sequence comprising at least 50 amino acids.

In a fifth aspect, the invention concerns a non-human eukaryotic host cell comprising:
a) a CRISPR nuclease, preferably a class II CRISPR nuclease, or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region, and
c) optionally a fusion protein comprising a Spo11 protein operably linked to a DNA binding domain, or a nucleic acid encoding said fusion protein,
wherein the CRISPR nuclease is not fused with a Spo11 protein and
wherein the activity of the CRISPR nuclease is repressed during the mitotic phase by
(i) placing the expression of said CRISPR nuclease and/or said guide RNAs under the control of a promoter inducible during the meiotic phase or a meiosis-specific promoter, and/or
(ii) expressing an inhibitor of CRISPR endonuclease activity during the mitotic phase, and/or
(iii) using a system triggering the degradation of the CRISPR nuclease during the mitotic phase, and/or
(iv) using CRISPR nuclease which is a variant of a wild-type CRISPR nuclease, said variant exhibiting a reduced endonuclease activity by comparison to said wild-type CRISPR nuclease.

In particular; in the method or the host cell according to the invention, when the cell is a plant cell, the CRISPR nuclease and/or the guide RNA(s) may be introduced into the plant cell, preferably into inflorescence, by a viral vector, preferably a viral vector derived from a Geminivirus.

Preferably, the cell envisioned by the invention is a yeast cell, a plant cell or a fungus cell. More preferably, the cell envisioned by the invention is a yeast cell or a plant cell, even more preferably a plant cell.

In particular, the cell may be a plant cell selected from the group consisting of rice, wheat, soybean, corn, tomato, onion, cucumber, lettuce, asparagus, carrot, turnip, Arabidopsis thaliana, millet, barley, rapeseed, cotton, grapevine, sugar cane, beet, cotton, sunflower, oil palm, coffee, tea, cocoa, chicory, bell pepper, chili, lemon, orange, nectarine, mango, apple, banana, peach, apricot, sweet potato, yams, almond, hazelnut, strawberry, melon, watermelon, olive, potato, zucchini, eggplant, avocado, cabbage, plum, cherry, pineapple, spinach, apple, tangerine, mandarin, grapefruit, pear, grape, clove, cashew, coconut, sesame, rye, hemp, tobacco, berries, raspberry, blackcurrant, peanut, castor beans, vanilla, poplar, eucalyptus, green foxtail, cassava, roses, tulips, orchids, rubber tree and geraniums.

Preferably, the cell is a tomato cell, a rice cell, a corn cell or a soja cell. Even more preferably, the cell is a tomato cell.

### BRIEF DESCRIPTION OF THE DRAWINGS

FIG. 1: TetON method to reduce the leaky activity of FnCpf1+crRNA expression in mitotic cells. (A) Schematic representation of the recombination assay in the GAL2 region. The flanking NatMX and HphMX cassettes were inserted upstream and downstream of the GAL2 gene in trans configuration. Crossover between these markers generate two reciprocal products, without these markers (RI) or carrying both markers (R2). The site targeted by SpCas9 and FnCpf1 is located in the GAL2 gene promoter (black bar). Genomic DNA digestion by XbaI makes parental (P1 and P2) and recombinant (R1 and R2) fragments of different sizes (7.3, 7.7, 6.3 and 8.7 kb, respectively). These fragments were detected by Southern blot using a DNA probe located in the GAL2 coding sequence. (B) In these experiments, FnCpf1 is expressed under the meiosis-specific REC8 promoter and the crRNA under the SNR52 promoter or a doxycycline-inducible SNR52 promoter (TetON system). The utilization of the TetON system (right panel) decreases the frequency of targeted DSB formation and recombinant molecules in mitotic cells. DSB formation at the GAL2 promoter was analyzed by Southern blot in cells harvested during mitotic growth and meiotic progression (TO-8h). Digestion of the genomic DNA by XbaI allows to visualize and quantify the P1, P2, R1 and R2 bands separated by their different size (FIG. 1A). At the left of the blot, the thick arrows indicate the expected location of the targeted FnCpf1 DSB1 and DSB2 sites on the P1 and P2 homologous chromosomes, respectively. The thin arrows indicate the parental (P1 and P2) and recombinant (R1 and R2) fragments. On the right, a map shows the open reading frames (open arrows indicate the transcriptional sense of the natural genes) and the position of the probe (hatched rectangle). The FnCpf1 target site is shown with black bars on each parental molecule. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

FIG. 2: Auxin-inducible degron system to eliminate SpCas9 expression in mitotic cells. In these experiments, SpCas9 was fused to an auxin-inducible degron. The auxin was added to the mitotic growth media (right panel) or not (left panel). DSB formation at the GAL2 promoter was analyzed by Southern blot in cells harvested during mitotic growth (Mito) and meiotic progression (TO-8h). The heterozygous markers NatMX and HphMX were introduced on either side of the targeted region in the parental chromosomes P1 and P2 as described in FIG. 1A. Digestion of the genomic DNA by XbaI allows to visualize and quantify the P1, P2, R1 and R2 bands separated by their different size (FIG. 1A). At the left of the blot, thick arrows indicate the targeted SpCas9 DSBs on the homologous chromosomes and thin arrows indicate the parental (P1 and P2) and recombinant (R1 and R2) fragments. On the right, a map shows the open reading frames (open arrows indicate the transcriptional sense) and the position of the probe (hatched rectangle). The SpCas9 target sites are shown as black bars on the homologous chromosomes. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

FIG. 3: Clustered targeting of dCas9-Spo11 in the GAL2 promoter enhanced DSB frequency in the targeted locus. In these experiments, the SpdCas9-Spo11 fusion is expressed under the constitutive ADH1 promoter and the diverse gRNA(s) expressed under the control of the RPR1 promoter. DSB formation at the GAL2 promoter was analyzed by Southern blot in cells harvested during meiotic progression (TO-8 h). The pGAL2 sites targeted by the sgRNA(s) are indicated at the top of the blot. The poly sgRNAs were released from the endogenous processing of synthetic polycistronic gene sequences (PGS), that encode gRNA-tRNA cassettes (see Materials & Methods). The 3 synthetic PGS encode gRNA-tRNA cassettes expressing mature gRNAs targeting the -A, -B and -D/E (pAS685), sgRNA-7,8 and 10 (pAS686) and sgRNA-4,5,6,8 and 9 (pAS687) genomic sites located within the GAL2 promoter. To express 2 different PGS in the same diploid strain, their sequences were integrated at the ARG4 locus (chromosome VIII), one on each homologous chromosome. At the left of the blot, a map shows the open reading frames (open arrows indicate the transcriptional sense) and the position of the probe (hatched rectangle). At the right of the blot, the arrows indicate the expected location of the targeted SpdCas9-Spo11-induced DSBs. The SpdCas9-Spo11 target sites are shown as black bars. Frequency of DSBs corresponds to the sum of the DSB signal detected in the GAL2 promoter compared to the total signal in the lane. Regarding the targeting of dCas9-Spo11 with the individual sgRNA-A, B or D/E, the mean of DSB frequencies observed with the 3 single sgRNA is indicated. The experiments were performed in diploid strains homozygous for the mutant sae2 allele, allowing the accumulation of unrepaired Spo11-dependent DSBs.

FIG. 4. Meiotic SpCas9-induced DSBs are resected. In these experiments, SpCAS9 is expressed under the meiosis-specific REC8 promoter and the sgRNA-pGAL2-D/E under the doxycycline-inducible RPR1 promoter (TetON system). DSB formation at the GAL2 promoter was analyzed by Southern blot in cells harvested during mitotic growth (Mito) and meiotic progression. Genomic DNA digested with XbaI. At the left of the blot, an arrow indicates the expected location of the targeted SpCas9 DSB on the chromosome XII. On the right, a map shows the open reading frames (open arrows indicate the transcriptional sense of the natural genes) and the position of the probe (hatched rectangle). The SpCas9 target site is shown as a black bar. DSB frequency corresponds to the radioactivity in the DSB band compared to the total signal in the lane.

FIG. 5. SpCas9 stimulates meiotic recombination in the targeted GAL2 region in SPO11 but not in spo11Δ cells. In these experiments, SpCAS9 is expressed under the meiosis-specific REC8 promoter and the sgRNA-pGAL2-D/E under the doxycycline-inducible RPR1 promoter (TetON system). (A) DSB formation at the GAL2 promoter was analyzed by Southern blot in cells harvested during mitotic growth (Mito) and meiotic progression. The heterozygous markers NatMX and HphMX were introduced on either side of the targeted region in the parental chromosomes P1 and P2 as described in FIG. 1A. Digestion of the genomic DNA by XbaI allows to visualize and quantify the P1, P2, R1 and R2 bands separated by their different size (FIG. 1A). At the left of the blot, the thick arrows indicate the expected location of the targeted SpCas9 DSB1 and DSB2 sites on the P1 and P2 homologous chromosomes, respectively. The thin arrows indicate the parental (P1 and P2) and recombinant (R1 and R2) fragments. On the right, a map shows the open reading frames (open arrows indicate the transcriptional sense of the natural genes) and the position of the probe (hatched rectangle). SpCas9 target site is shown as a black bar. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands. (B) Tetrads from diploid strains were dissected and spore viability was measured (%). The total number of dissected tetrads is reported in parentheses. Recombination rates were measured by examining the segregation of the NatMX and HphMX cassettes in the meiotic products upon dissection of 191 WT and 228 CAS9 tetrads, respectively. (see FIG. 1A describing the schematic representation of the recombination assay in the GAL2 region).

FIG. 6. Co-expression of three distinctive gRNAs allows poly-targeting of SpCas9 and FnCpf1 in the GAL2 promoter. In these experiments, SpCAS9 and FnCPF1 are expressed under the meiosis-specific REC8 promoter. Expression of sgRNAs and crRNAs is controlled by the RPR1 and SNR52 promoters, respectively. DSB formation at the GAL2 promoter was analyzed by Southern blot in cells harvested during mitotic growth (Mito) and meiotic progression. Genomic DNA digested with XbaI. At the left of the blot, an arrow indicates the expected location of the targeted SpCas9 and FnCpf1 DSBs on the chromosome XII. The asterisk indicates a cross hybridizing band. On the right, a map shows the open reading frames (open arrows indicate the transcriptional sense of the natural genes) and the position of the probe (hatched rectangle). The SpCas9 and FnCpf1 target sites are shown as black bars. DSB frequency corresponds to the radioactivity in the DSB band compared to the total signal in the lane.

FIG. 7. Poly-targeting of SpCas9 and FnCpf1 in the GAL2 promoter enhanced meiotic recombination in the targeted GAL2 region. (A) In these experiments, SpCAS9 and FnCPF1 are expressed under the meiosis-specific REC8 promoter. Expression of sgRNAs and crRNAs is controlled by the RPR1 and SNR52 promoters, respectively. DSB formation at the GAL2 promoter was analyzed by Southern blot in cells harvested during mitotic growth (Mito) and meiotic progression. The heterozygous markers NatMX and HphMX were introduced on either side of the targeted region in the parental chromosomes P1 and P2 as described in FIG. 1A. Digestion of the genomic DNA by XbaI allows to visualize and quantify the P1, P2, R1 and R2 bands separated by their different size (FIG. 1A). At the left of the blot, the arrows indicate the expected location of the targeted CRISPR DSB-A, -B, -D/E sites on the P1 and P2 homologous chromosomes, respectively. On the right, a map shows the open reading frames (open arrows indicate the transcriptional sense of the natural genes) and the position of the probe (hatched rectangle). SpCas9 and FnCpf1 target sites are shown as black bars. The arrows indicate the parental (P1 and P2) and recombinant (R1 and R2) fragments. (B) DSB frequency corresponds to the sum of the radioactivity in the DSB-A, -B, -D/E bands compared to the total signal in the lane. (C) Frequency of recombinant molecules was measured as the percentage of radioactivity in R1+R2 relative to the total amount of radioactivity in the parental P1+P2 plus recombinant R1+R2 bands.

FIG. 8. Comparison of SpCas9 poly-targeting and co-targeting of SpCas9 plus SpdCas9-Spo11 in the GAL2 region. (A) In these experiments, SpCAS9 and SpdCAS9-SPO11 are expressed under the meiosis-specific REC8 and the constitutive ADH1 promoters, respectively. Expression of sgRNAs is controlled by doxycycline-inducible RPR1 promoter (TetON system). The sgRNAs-1, -2, -3, -5, -6, -7 were released from the endogenous processing of a synthetic polycistronic gene sequence (PGS), that encodes tRNA-gRNA cassettes (see Materials & Methods). The 7 sgRNAs targeting the GAL2 gene sequence were expressed from the pAS704 plasmid that carries the synthetic PGS with 6 sgRNAs and the sgRNA-4 expression cassette. DSB formation in the GAL2 gene was analyzed by Southern blot in cells harvested during mitotic growth (Mito) and meiotic progression. The heterozygous markers NatMX and HphMX were introduced on either side of the targeted region in the parental chromosomes P1 and P2 as described in FIG. 1A. Digestion of the genomic DNA by XbaI allows to visualize and quantify the P1, P2, R1 and R2 bands separated by their different size (FIG. 1A). At the right of the blot, the arrows indicate the expected location of the targeted DSB-1 to -7 sites on the P2 homologous chromosome (DSB-1 to -7 on the P1 homologous chromosome generate DNA fragments that are too small to be detected on this blot). On the right, a map shows the open reading frames (open arrows indicate the transcriptional sense of the natural genes) and the position of the probe (hatched rectangle). Cas9 and dCas9 target sites are shown as black bars. At the left of the blot, arrows indicate the parental (P1 and P2) and recombinant (R1 and R2) fragments. (B) DSB frequency corresponds to the sum of the radioactivity in the DSB-1 to-7 bands compared to the total signal in the lane. (C) Frequency of recombinant molecules was measured as the percentage of radioactivity in R1+R2 relative to the total amount of radioactivity in the parental P1+P2 plus recombinant R1+R2 bands.

FIG. 9. Poly-targeting of SpCas9 enhanced meiotic recombination in the targeted pericentromeric GAL3 region. (A) Schematic representation of the recombination assay in the GAL3 region. The TRP1 gene was disrupted by the hisG sequence and the HphMX cassette was inserted downstream of the GAL3 gene in trans configuration regarding the TRP1 marker. Crossover between the TRP1 and HphMX markers generate two reciprocal products, without these markers (R2) or carrying both markers (R1). The sites targeted by SpCas9 are located in the GAL3 gene promoter (black bar). Genomic DNA digestion by SpeI and PvuII makes parental (P1 and P2) and recombinant (R1 and R2) fragments of different sizes (5.4, 7.9, 6.8 and 6.5 kb, respectively). These fragments were detected by Southern blot using a DNA probe located in the GAL3 coding sequence. (B) In these experiments, SpCAS9 is expressed under the meiosis-specific REC8 and promoter. Expression of sgRNAs is controlled by doxycycline-inducible RPR1 promoter (TetON system). The sgRNAs-1, -2, -3, -5, -6, -7 were released from the endogenous processing of a synthetic polycistronic gene sequence (PGS), that encodes sgRNA-tRNA cassettes (see Materials & Methods). The 6 sgRNAs targeting the GAL3 promoter were expressed from the pAS705 plasmid, that carries the synthetic PGS. DSB formation in the GAL3 promoter was analyzed by Southern blot in cells harvested during mitotic growth (Mito) and meiotic progression. The heterozygous markers TRP1 and HphMX flanked the targeted region in the parental chromosomes P1 and P2 as described in FIG. 1A. Digestion of the genomic DNA by SpeI and PvuII allows to visualize and quantify the P1, P2, R1 and R2 bands separated by their different size (FIG. 9A). On the blot, the arrows indicate the expected location of the targeted DSB-1 to -7 sites on the P1 and P2 homologous chromosomes, respectively. On the right, a map shows the open reading frames (open arrows indicate the transcriptional sense of the natural genes) and the position of the probe (hatched rectangle). SpCas9 target sites are shown as black bars. At the right of the blot, arrows indicate the parental (P1 and P2) and recombinant (R1 and R2) fragments. (C) DSB frequency corresponds to the sum of the radioactivity in the DSB-4 or DSB-1, -2, -3, -5, -6, -7 bands compared to the total signal in the lane. (D) Frequency of recombinant molecules was measured as the percentage of radioactivity in R1+R2 relative to the total amount of radioactivity in the parental P1+P2 plus recombinant R1+R2 bands.

### DETAILED DESCRIPTION OF THE INVENTION

In a first aspect, the present invention relates to a method for inducing targeted meiotic recombination in an eukaryotic cell, in particular a non-human eukaryotic cell, said method comprising introducing into said eukaryotic cell a) a CRISPR nuclease, or a nucleic acid encoding said nuclease, and b) one or more guide RNAs or one or more nucleic acids encoding said guide RNAs, said guide RNAs comprising an RNA structure for binding to the CRISPR nuclease and a sequence complementary to a targeted chromosomal region of said cell and inducing said cell to enter meiotic prophase I.

Said method is further designed to preserve the integrity, during the mitotic phase, of the chromosomal region targeted by the guide RNAs during the meiotic phase of the cell cycle and/or to increase the probability of having at least one preserved site recognized by at least one guide RNA in the chromosomal region targeted during the meiotic phase.

In an embodiment, the chromosomal region targeted by the guide RNAs is of less than 30 kb, preferably less than 25 kb, even more preferably less than 20 kb. In particular, the chromosomal region targeted by the guide RNAs is of between 0.5 and 30 kb long, preferably between 0.5 and 20 kb.

In particular, the chromosomal region targeted by the guide RNAs may be a pericentromeric region. By “pericentromeric region” is meant a specific chromosomal region that expand from both sides of the centromere, where the kinetochore assembles to ensure the faithful segregation of sister chromatids during mitosis and meiosis. A pericentromeric region containing repetitive sequences and transposable elements surrounds the centromere and adopts a particular chromatin state characterized by specific histone variants and post-translational modifications and forms a transcriptionally repressive chromosomal environment. Pericentromeric regions are typically enriched in cohesion and condensin. Their size varies according to the organisms. In particular, the pericentromeric region may be a region within 30 kb and 5 Mb from the centromere. More particularly, the pericentromeric region may be a region within 30 kb and 50 kb from the centromere in yeasts and within 5 Mb from the centromere in plants.

The integrity of the chromosomal region targeted by the guide RNAs may be preserved by repressing the activity of the CRISPR nuclease during the mitotic phase. In particular, this activity may be repressed during the mitotic phase (i) by placing the expression of the CRISPR nuclease and/or guide RNAs under the control of a promoter inducible during the meiotic phase or a meiosis-specific promoter, (ii) by expressing an inhibitor of CRISPR endonuclease activity during the mitotic phase, (iii) by using a system triggering the degradation of the CRISPR nuclease during the mitotic phase and/or (iv) by using a CRISPR nuclease which is a variant of a wild-type CRISPR nuclease, said variant exhibiting a reduced endonuclease activity by comparison to said wild-type CRISPR nuclease.

Alternatively or in addition, the probability of having at least one preserved site recognized by at least one guide RNA in the chromosomal region targeted during the meiotic phase may be increased by using a plurality of guide RNAs targeting the same chromosomal region.

Each of these techniques may be used alone or in combination with any of the others.

In an embodiment, the method of the invention for inducing targeted meiotic recombination in a eukaryotic cell comprises
introducing into said cell:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
wherein the activity of the CRISPR nuclease is repressed during the mitotic phase.

Preferably, the method of the invention for inducing targeted meiotic recombination in a cell comprises:
introducing into said cell:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
wherein the activity of the CRISPR nuclease is repressed outside of meiosis,
wherein the CRISPR nuclease is not fused with a Spo11 protein, and
wherein the cell is a yeast cell, a plant cell or a fungus cell.

As mentioned above, the activity of the CRISPR nuclease during the mitotic phase may be repressed using several distinct strategies, in particular (i) by placing the expression of the CRISPR nuclease and/or guide RNAs under the control of a promoter inducible during the meiotic phase or a meiosis-specific promoter, (ii) by expressing an inhibitor of CRISPR endonuclease activity during the mitotic phase, (iii) by using a system triggering the degradation of the CRISPR nuclease during the mitotic phase and/or (iv) by using a CRISPR nuclease which is a variant of a wild-type CRISPR nuclease, said variant exhibiting a reduced endonuclease activity by comparison to said wild-type CRISPR nuclease. Each of these strategies may be used alone or in combination with any of the others.

The cell is a eukaryotic cell, preferably a non-human eukaryotic cell. As used herein, the term “eukaryotic cell”, refers to a yeast cell, plant cell, fungus cell or an animal cell, preferably a yeast cell, plant cell, fungus cell or a non-human mammalian cell such as a mouse or rat cell, or an insect cell.

In some embodiments, when the cell is an animal cell, the gRNAs disclosed herein does not target genes and genomic loci that can cause suffering in animals.

In a preferred embodiment, the eukaryotic cell is a plant cell, in particular a plant cell of agronomic, horticultural, pharmaceutical or cosmetic interest, including vegetables, fruits, herbs, flowers, trees and shrubs. Preferably the plant cell is selected from monocotyledonous plants and dicotyledonous plants, more preferably selected from the group consisting of rice, wheat, soybean, corn, tomato, onion, cucumber, lettuce, asparagus, carrot, turnip, Arabidopsis thaliana, millet, barley, rapeseed, cotton, grapevine, sugar cane, beet, cotton, sunflower, oil palm, coffee, tea, cocoa, chicory, bell pepper, chilli, lemon, orange, nectarine, mango, apple, banana, peach, apricot, sweet potato, yams, almond, hazelnut, strawberry, melon, watermelon, olive, potato, zucchini, eggplant, avocado, cabbage, plum, cherry, pineapple, spinach, apple, tangerine, mandarin, grapefruit, pear, grape, clove, cashew, coconut, sesame, rye, hemp, tobacco, berries, raspberry, blackcurrant, peanut, castor beans, vanilla, poplar, eucalyptus, green foxtail, cassava and horticultural plants such as roses, tulips, orchids, rubber tree and geraniums. In particular, the plant cell can be selected from the group consisting of rice, wheat, soybean, corn, tomato, onion, cucumber, lettuce, asparagus, carrot, turnip, Arabidopsis thaliana, barley, rapeseed, cotton, grapevine, sugarcane, beet, cotton, sunflower, palm olive, coffee tea, cocoa, chicory, bell pepper, chili, lemon, orange, nectarine, mango, apple, banana, peach, apricot, sweet potato, yams, almonds, hazelnuts, strawberries, melons, watermelons, olives, and horticultural plants such as roses, tulips, orchids, rubber tree and geraniums. More preferably the plant cell is selected from the group consisting of tomato, carrot, lettuce, potato, strawberry, raspberry, grapes, apple, coffee, cocoa, corn, soybean, canola, rice, cotton, wheat, oat, hemp, barley, okra and chickpea.

Preferably, the cell is a tomato cell, a rice cell, a corn cell or a soja cell. Even more preferably, the cell is a tomato cell.

In another preferred embodiment, the eukaryotic cell is a yeast cell, in particular a yeast of industrial interest. Examples of yeasts of interest include, but are not limited to, yeasts of the genus Saccharomyces sensu stricto, Schizosaccharomyces, Yarrowia, Hansenula, Kluyveromyces, Pichia or Candida, as well as hybrids obtained from a strain belonging to one of these genera. Preferably, the yeast of interest belongs to the genus Saccharomyces. preferably a yeast selected from the group consisting of Saccharomyces cerevisiae, Saccharomyces bayanus, Saccharomyces castelli, Saccharomyces eubayanus, Saccharomyces kluyveri, Saccharomyces kudriavzevii, Saccharomyces mikatae Saccharomyces uvarum, Saccharomyces paradoxus, Saccharomyces pastorianus (also called Saccharomyces carlsbergensis), and hybrids obtained from at least one strain belonging to one of these species such as for example a hybrid S. cerevisiae/S. paradoxus hybrid or an S. cerevisiae/S. uvarum hybrid, even more preferably, said yeast is Saccharomyces cerevisiae.

In another embodiment, the eukaryotic cell is a fungal cell, in particular a fungal cell of industrial interest. Examples of fungi include, but are not limited to, filamentous fungi cells. Filamentous fungi include fungi belonging to the subdivisions Eumycota and Oomycota. The cells of filamentous fungi may be selected from the group consisting of cells of Trichoderma, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Sordaria, Talaromyces, Thermoascus, Thielavia, Tolypocladium or Trametes.

The CRISPR system (“Clustered Regularly Interspaced Short Palindromic Repeats”) is a defense system in bacteria and archaea against foreign DNA. These short fragments corresponding to the infectious agent are inserted into a series of CRISPR repeats and are used as CRISPR RNA guides (crRNA) to target the infectious agent in subsequent infections. This system is essentially based on the association of a CRISPR-associated endonuclease (Cas) and a guide RNA (gRNA or sgRNA) responsible for cleavage site specificity. It leads to DNA double-strand breaks (DSBs) at the sites targeted by the CRISPR system.

As used herein, the term “guide RNA” or “gRNA” refers to an RNA molecule capable of interacting with the CRISPR nuclease. Each gRNA comprises two regions: a first region which is complementary to the target chromosomal region and a second region which is required for interaction with the CRISPR nuclease. In particular, a gRNA is a piece of RNA that functions as a guide for RNA- or DNA-targeting enzymes, such as CRISPR nuclease, with which it forms complexes.

In embodiments wherein several gRNAs are used, the first regions may vary according to the targeted chromosomal sequences. On the other hand, the second regions of the gRNAs may be identical or different, preferably are identical.

The first region of the gRNA, which is complementary to a target chromosomal sequence, generally comprises between 10 and 25 nucleotides. The second region of the gRNA has a stem-loop (or hairpin) structure. The lengths of the stem and the loop may vary. Preferably, the loop has a length of 3 to 10 nucleotides and the stem a length of 6 to 20 nucleotides. The stem may optionally have mismatched regions (forming “bulges”) of 1 to 10 nucleotides. Preferably, the total length of this region is from 50 to 100 nucleotides. The total length of a gRNA is typically between 30 and 150 nucleotides. Preferably, the total length of the guide RNA is comprised between 65 and 105 nucleotides.

The gRNA is preferably formed of a single RNA molecule comprising the two regions. Alternatively, the gRNA may be formed of two distinct RNA molecules, the first molecule comprising the first region and half of the stem of the second region, and the second molecule comprising the second half of the stem of the gRNA. Thus, the pairing of the two RNA molecules by their complementary sequences at the stem, forms a functional gRNA.

The gRNAs can be introduced into the eukaryotic cell as mature gRNA molecules, as precursors, or as one or more nucleic acids encoding said gRNAs.

When the gRNA(s) are introduced into the cell directly as RNA molecules (mature or precursors), these gRNAs may contain modified nucleotides or chemical modifications allowing them, for example, to increase their resistance to nucleases and thus to increase their lifespan in the cell. They may notably include at least one modified or non-natural nucleotide such as, for example, a nucleotide comprising a modified base, such as inosine, methyl-5-deoxycytidine, dimethylamino-5-deoxyuridine, deoxyuridine, diamino-2,6-purine, bromo-5-deoxyuridine or any other modified base allowing hybridization. The gRNAs used according to the invention may also be modified at the internucleotide bond such as for example phosphorothioates, H-phosphonates or alkyl-phosphonates, or at the backbone such as for example alpha-oligonucleotides, 2′-O-alkyl-riboses or peptide nucleic acid (PNA) (Egholm et al., 1992, J. Am. Chem. Soc., 114, 1895-1897).

The gRNAs may be natural RNA, synthetic RNA, or RNA produced by recombination techniques. These gRNAs may be prepared by any methods known to a person skilled in the art such as, for example, chemical synthesis, in vivo transcription or amplification techniques.

A person skilled in the art can, by using well-known techniques, readily define the sequence and the structure of the gRNAs according to the chromosomal sequence to be targeted (see for example the article by Di Carlo et al., 2013, Nucleic Acids Res., 41, 4336-4343).

There are five main types of CRISPR systems that differ in the repertoires of CRISPR-associated genes, the organization of Cas operons and the structure of repeats. These five types of systems have been divided into two classes: class I comprising types I, III and IV that use a multimeric crRNA effector module and class II comprising types II, V and VI that use a monomeric crRNA effector module. Type II includes types II-A,II-B,II-C,II-C, type V includes types V-A, V-B, V-C, V-D, V-E, V-U1, V-U2, V-U3, V-U4, V-U5 and type VI includes types VI-A,VI-B1,VI-B2,VI-C,VI-D.

In preferred embodiments, the CRISPR nuclease is a class II nuclease, in particular a class II and type II, V or VI nuclease, preferably a class II and type II or type V nuclease.

Class II and Type II CRISPR nucleases, predominantly represented by Cas9 and Csn2 nucleases, comprise a small trans-acting RNA called tracrRNA (“trans-acting crRNA”) that pairs with each pre-crRNA repeat (“CRISPR RNA”) to form a double-stranded RNA [tracrRNA:crRNA] that is cleaved by RNase III in the presence of the endonuclease.

The class II and type VI CRISPR nucleases are represented by the C13a (previously known as C2c2), C13b and C13c nucleases. The CRISPR-C13 system was discovered in the bacterium Leptotrichia shahii (Abudayyeh et al., 2016, Science; 353, aaf5573) and is analogous to the CRISPR-Cas9 system. However, unlike Cas9, which targets DNA, C13 proteins target and cleave single-stranded RNA.

Class II and type V CRISPR-nucleases, are mainly represented by the Cpf1 nuclease (also known as Cas12a) identified in Francisella novicida (Zetsche et al., 2015, Cell, 163, 759-771), and the C2c1 (also known as Cas12b) and C2c3 nucleases identified in Alicyclobacillus acidoterrestris (Shmakov et al., 2015, Mol. Cell, 60, 385-397). A functional CRISPR-Cpf1 system does not require a tracrRNA but only a crRNA. In particular, a 42-44 nucleotide crRNA with a direct repeat sequence of about 19 nucleotides followed by a proto-spacer sequence of 23-25 nucleotides is sufficient to guide the Cpf1 endonuclease to the target nucleic acid.

Preferably, the CRISPR-nuclease is a class II CRISPR nucleases, in particular a CRISPR nuclease selected from the group consisting of wild-type Cas1, Cas1B, Cas2, Cas3, Cas4, Cas5, Cas6, Cas7, Cas8, Cas9 (also known as Csn1 and Csx12), Cas10, Cas11 (SS), Cas12a (Cpf1), Cas12b (C2c1), Cas12c (C2c3), Cas12d (CasY), Cas12e (CasX), Cas12f (Cas14a), Cas12j (CasΦ), C2c4, C2c8, C2c5, C2c10, C2c9, Cas13a (C2c2), Cas13b (C2c6), Cas13c (C2c7) Cas13d, Csa5, Csc1, Csc2, Cse1, Cse2, Csy1, Csy2, Csy3, Csf1, Csf2, Csf3, Csf4, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, and orthologues, variants or functional fragments thereof exhibiting nuclease activity and capable of interacting with the guide RNAs. In particular, the CRISPR-nuclease may be selected from the group consisting of wild-type Cas1, Cas1B, Cas2, Cas3, Cas4, Cas5, Cas6, Cas7, Cas8, Cas9 (also known as Csn1 and Csx12), Cas1O, Cas11 (SS), Cas12a (Cpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7) Cas13d, Csa5, Csc1, Csc2, Cse1, Cse2, Csy1, Csy2, Csy3, Csf1, Csf2, Csf3, Csf4, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, and orthologues, variants or functional fragments thereof exhibiting nuclease activity and capable of interacting with the guide RNAs.

The CRISPR nuclease may also be a miniature CRISPR nuclease engineered from said CRISPR nucleases such as CasMINI (engineered from the type V-F Cas12f (Cas14)) described in Xu et al., 2021, Mol. Cell, 81, 4333-4345. Indeed, the CRISPR-Cas system used in the present invention may be a miniature CRISPR-Cas system engineered from known CRISPR nuclease systems by guide RNA and protein engineering as described in Xu et al., 2021, Mol. Cell, 81, 4333-4345.

Preferably, the CRISPR nuclease is selected from the group consisting of wild-type Cas1, Cas2, Cas9, Csn2, Cas13, C13a (C2c2), C13b, C13c, Cas12, C12a (Cpf1), C2c1, and C2c3, and orthologues, variants or functional fragments thereof exhibiting nuclease activity and capable of interacting with the guide RNAs.

More preferably, the CRISPR nuclease is selected from the group consisting of wild-type Cas9 or Cpf1 and orthologues, variants or functional fragments thereof exhibiting nuclease activity and capable of interacting with the guide RNAs.

In one embodiment, the CRISPR nuclease is selected from the group consisting of wild-type Cas9 and orthologues, variants or functional fragments thereof exhibiting nuclease activity and capable of interacting with the guide RNAs.

As used herein, the term “variant” refers to an enzyme which is derived from a CRISPR nuclease and comprises an alteration, i.e., a substitution, insertion, and/or deletion, at one or more (e.g., several) positions. The term “deletion”, used in relation to a position or an amino acid, means that the amino acid in the particular position has been deleted or is absent. The term “insertion”, used in relation to a position or amino acid, means that one or more amino acids have been inserted or are present adjacent to and immediately following the amino acid occupying the particular position. The variant may be obtained by various techniques well known in the art. In particular, examples of techniques for altering the DNA sequence encoding the wild-type protein, include, but are not limited to, site-directed mutagenesis, random mutagenesis and synthetic oligonucleotide construction.

A variant of CRISPR nuclease exhibits CRISPR nuclease activity and is capable of interacting with the guide RNAs. Said variant has preferably at least 70%, more preferably at least 80%, and even more preferably at least 90%, 95%, 96%, 97%, 98% or 99%, sequence identity to the parent CRISPR nuclease.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. Preferably, for purposes herein, % amino acid sequence identity values refers to values generated using the BLAST (Basic Local Alignment Search Tool) algorithm, wherein all search parameters are set to default values.

As used herein, the term “functional fragment” or “fragment” refers to a fragment of a CRISPR nuclease, comprising at least 100, 150, 200, 250, 300, 350, 400, 450 or 500 contiguous amino acids of said nuclease, and retaining the enzymatic activity of the entire polypeptide, i.e. exhibiting CRISPR nuclease activity and being capable of interacting with the guide RNAs.

The term “ortholog” or “orthologous protein” as used herein refers a functional counterpart (i.e. exhibiting CRISPR nuclease activity) of a protein in another species. Orthologous proteins are similar to each other because they originated from a common ancestor. Sequence differences between the orthologs are thus the result of speciation. The orthologous sequences can be encompassed in longer or shorter isoforms. Methods for identification of orthologous proteins are well known in the art.

The Cas9 nuclease as used in the present invention may be any known Cas9 nuclease or can be obtained from any known Cas9 nuclease (Makarova et al., 2008, Nat. Rev. Microbiol., 9, 466-477). Examples of Cas9 nucleases that can be used in the present invention include, but are not limited to, the Cas9 from Streptococcus pyogenes, Streptococcus thermophilus, Streptococcus sp., Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicellulosiruptor bescii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsonii, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Other Cas9 that can be used in the present invention are described in Makarova et al. (Makarova et al., 2008, Nat. Rev. Microbiol., 9, 466-477) and have been widely described in the literature.

In particular, examples of Cas9 nucleases include, but are not limited to, the Cas9 nucleases of Streptococcus pyogenes (SpCAS9, Uniprot accession number: Q99ZW2; SEQ ID NO: 1), Streptococcus thermophilus (St1Cas9, St3Cas9, Uniprot accession number: G3ECR1; SEQ ID NO: 2), Staphylococcus aureus (SaCas9, Uniprot accession number: J7RUA5; SEQ ID NO: 3), Campylobacter jejuni (CjCas9, Uniprot accession number: Q0P897; SEQ ID NO: 4), Francisella novicida (FnCas9, Uniprot accession number: A0Q5Y3; SEQ ID NO: 5) and Neisseria meningitidis (NmCas9, Uniprot accession number: X5EPV9; SEQ ID NO: 6).

In a particular embodiment, the CRISPR nuclease comprises, or consists of, an amino acid sequence selected from the group consisting of SEQ ID NO: 1 to 6 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 1 to 6. Preferably, the CRISPR nuclease comprises, or consists of, an amino acid sequence selected from the group consisting of SEQ ID NO: 1 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 1.

More preferably, the CRISPR nuclease comprises, or consists of, the sequence set forth in SEQ ID NO: 1.

In another embodiment, the CRISPR nuclease is selected from the group consisting of Cpf1 and orthologues, variants or functional fragments thereof exhibiting nuclease activity and capable of interacting with the guide RNAs.

The Cpf1 nuclease as used in the present invention may be any known Cpf1 nuclease or can be obtained from any known Cpf1 nuclease. Examples of Cpf1 nucleases that can be used in the present invention include, but are not limited to, the Cpf1 nucleases from bacteria of the genus Prevotella, Moraxella, Leptospira, Lachnospiraceae, Francissela, Candidatus, Eubacterium, Parcubacteria, Peregrinibacteria, Acidmicococcus and Prophyromonas.

In particular, examples of Cpf1 nucleases include, but are not limited to, the Cpf1 nucleases of Parcubacteria GWC2011_GWC2_44_17 (PbCpf1, Genbank accession number: KKT48220.1; SEQ ID NO: 7), Peregrinibacteria GW2011_GWA_33_10 (PeCpf1, Genbank accession number: KKP36646. 1; SEQ ID NO: 8), Acidaminococcus sp. (UniProt accession number: U2UMQ6; SEQ ID NO: 9), Prophyromonas macacae (PmCpf1, Genbank accession number: WP_018359861. 1; SEQ ID NO: 10), Prophyromonas crevioricanis (PcCpf1, Genbank accession number WP_036890108. 1; SEQ ID NO: 11), Smithella species SC K08D17 (UniProt accession number: A0AOC1NU26 SEQ ID NO: 12), Prevotella disiens (PdCpF1, Genbank accession number: WP_004356401. 1; SEQ ID NO: 13), Moraxella bovoculi 237 (MbCpf1, Genbank accession number: KDN25524. 1; SEQ ID NO: 14), Leptospira inadai (LiCpf1, Genbank accession number: WP_020988726.1, SEQ ID NO: 15), Lachnospiraceae bacterium MA2020 (LbCpf1, UniProt accession number: A0A182DWE3; SEQ ID NO: 16), Francisella novicida U112 (FnCPF1, UniProt accession number: A0Q7Q2; SEQ ID NO: 17). Preferably, the nuclease envisioned herein is a Cpf1 protein, from Francisella novicida, in particular such as described under SEQ ID NO: 17. Of course, these examples are not limiting and any known Cpf1 protein can be used in the process according to the invention.

In a particular embodiment, the CRISPR nuclease comprises, or consists of, an amino acid sequence selected from the group consisting of SEQ ID NO: 7 to 17 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 7 to 17. Preferably, the CRISPR nuclease comprises, or consists of, an amino acid sequence selected from the group consisting of SEQ ID NO: 17 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 17. More preferably, the CRISPR nuclease comprises, or consists of, the sequence set forth in SEQ ID NO: 17.

In another embodiment, the CRISPR nuclease is selected from the group consisting of Cas13 nucleases, i.e. Cas13a (C2c2), Cas13b and Cas13c, and orthologues, variants or functional fragments thereof exhibiting nuclease activity and capable of interacting with the guide RNAs.

The Cas13 nuclease as used in the present invention may be any known Cas13 nuclease or can be obtained from any known Cas13 nuclease. Examples of Cas13 nucleases that can be used in the present invention include, but are not limited to, the Cas13 nucleases from Herbinix hemicellulosilytica (Cas13a, GenBank accession number: WP_103203632.1; SEQ ID NO: 20), Lachnospiraceae bacterium (Cas13a, GenBank accession number: WP_022785443.1; SEQ ID NO: 21) or Leptotrichia wadei (Cas13a, GenBank accession number: WP_021746003.1; SEQ ID NO: 22).

In a particular embodiment, the CRISPR nuclease comprises, or consists of, an amino acid sequence selected from the group consisting of SEQ ID NO: 20 to 22 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 20 to 22.

In a particular embodiment, the CRISPR nuclease comprises, or consists of, an amino acid sequence selected from the group consisting of SEQ ID NO: 1 to 17 and 20-22 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 1 to 17 and 20-22.

In a preferred embodiment, the CRISPR nuclease comprises, or consists of, an amino acid sequence selected from the group consisting of SEQ ID NO: 1 to 17 and variants or functional fragments thereof exhibiting CRISPR nuclease activity and capable of interacting with the guide RNAs and having at least 70%, preferably at least 80%, more preferably at least 90%, 95%, 96%, 97%, 98% or 99%, sequence identity to any of SEQ ID NO: 1 to 17.

In preferred embodiments, the CRISPR nuclease is not fused with a Spo11 protein.

In particular, in preferred embodiments, the CRISPR nuclease is not fused with:
a Spo11 protein or a variant or fragment thereof as defined below; and/or
a Spo11 partner as defined below, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; and/or
a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; and/or
a DNA binding domain; and/or
a protein/amino acid sequence comprising at least 50 amino acids.

In particular embodiments, the CRISPR nuclease is not fused with a Spo11 protein or a variant or fragment thereof as defined below.

In particular embodiments, the CRISPR nuclease is not fused with a Spo11 protein partner as defined below, in particular such as a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto.

In some embodiments, the CRISPR nuclease is not fused with a topoisomerase, preferably selected from the TOPOVIB or MTOPOVIB family or a variant or orthologue thereof, preferably said variants having at least 80, 85, 90, 95, 96, 97, 98, or at least 99% sequence identity thereto.

In some embodiments, the CRISPR nuclease is not fused with a protein/amino acid sequence comprising at least 50 amino acids.

The CRISPR nuclease has a nuclease activity that allows DNA cleavage at a site targeted by a guide RNA. As used herein, the term “nuclease activity” refers to the enzymatic activity of an endonuclease that has an active site for creating breaks or cuts within DNA or RNA chains, preferably double-strand breaks in DNA (DSBs). The ability of a CRISPR nuclease to induce breaks within DNA chains (DNA endonuclease activity), in particular double-strand breaks, can be easily tested by the person skilled in the art by means of conventional techniques such as Southern blot hybridization of DNA, DNA sequencing or using the technique described in Zetsche, et al., 2015, Cell, 163, 759-771.

The activity of the CRISPR nuclease is repressed outside of meiosis, preferably outside of meiosis prophase, particularly during the mitotic phase. By “repressed during the mitotic phase” or “repressed outside of meiosis” it means that the CRISPR nuclease has a weak or no activity during the mitotic phase or has a decreased activity during the mitotic phase by comparison to the meiotic phase. The CRISPR nuclease is thus active during meiosis and the activity of the CRISPR nuclease during the mitotic phase may be repressed any method described herein, i.e. i) by placing the expression of the CRISPR nuclease and/or guide RNAs under the control of a promoter inducible during the meiotic phase or a meiosis-specific promoter, (ii) by expressing an inhibitor of CRISPR endonuclease activity during the mitotic phase, (iii) by using a system triggering the degradation of the CRISPR nuclease during the mitotic phase and/or (iv) by using a CRISPR nuclease which is a variant of a wild-type CRISPR nuclease, said variant exhibiting a reduced endonuclease activity by comparison to said wild-type CRISPR nuclease.

Preferably, the CRISPR nuclease is active at least during meiosis prophase I. Particularly, the CRISPR nuclease is only active, mainly active or most active during meiosis, preferably during meiosis prophase I.

In some embodiments, the activity of the CRISPR nuclease is repressed during the mitotic phase by using CRISPR nuclease which is a variant of a wild-type CRISPR nuclease, in particular a variant of a wild-type CRISPR nuclease such as described above, said variant exhibiting a reduced endonuclease activity by comparison to said wild-type CRISPR nuclease. This reduced endonuclease activity allows limiting DSBs during the mitotic phase and reparation via the non-homologous end-joining (NHEJ) pathway. This reduced activity is nevertheless sufficient to induce targeted recombination during meiosis. In particular, said CRISPR nuclease variant may comprise a mutated catalytic site, the mutation negatively impacting the nuclease or hydrolytic capacity of the protein but not impacting its ability to interact with the guide RNA and to recognize the targeted region of the nucleic acid. Preferably, said variant exhibits at least 80%, 85%, 90%, 95%, 96%, 97%, 98%, or at least 99% identity to the wild-type CRISPR nuclease and reduced nuclease activity.

By “reduced nuclease activity” is meant a reduced nuclease activity in comparison with the nuclease activity of the wild-type protein from which the variant is derived. Preferably, the nuclease activity of the variant is reduced by at least 50, 55, 60, 65, 70, 75, 80, 85, 90%, compared to the nuclease activity of the wild-type protein. Such variants are thus less efficient to generate double-strand breaks.

In such variant having a reduced nuclease activity, the ability to interact with guide RNAs and recognize the targeted region of the nucleic acid is maintained. The ability of a protein to interact with the guide RNA and to recognize the targeted region of the nucleic acid can be easily tested by the person skilled in the art, in particular by conventional techniques such as chromatin immunoprecipitation (ChIP) with an antibody recognizing the protein and its localization on the DNA, by PCR or sequencing.

In a particular embodiment, the CRISPR nuclease is a variant of a CRISPR class II nuclease having reduced nuclease activity, preferably a variant of a wild-type CRISPR class II and type II, V, or VI nuclease, more preferably a variant of a wild-type Cas9 or Cpf1 nuclease having reduced nuclease activity.

Generally, Cas9 proteins comprise two nuclease domains: a domain related to a RuvC domain and a domain related to an HNH domain. These two domains cooperate to create DNA double-strand breaks (Jinek et al., 2012, Science, 337: 816-821). Each of these nuclease domains can be inactivated by deletion, insertion or substitution according to techniques well-known to a person skilled in the art such as directed mutagenesis, PCR mutagenesis or total gene synthesis.

Example of Cas9 mutants exhibiting a reduced in vitro cleavage rate were disclosed in Bratovic et al. (Bratovic et al., 2020, Nat. Chem. Biol., 16, 587-595), such as Cas9 mutants exhibiting R63A, R66A, R69A, R70A, R71A, R74A or R78A single mutation in SEQ ID NO: 1, and in Jinek et al. (Jinek et al., 2012, Science, 337: 816-821) such as Cas9 mutants exhibiting D10A or H840A mutation in SEQ ID NO: 1.

In a particular embodiment, the variant of Cas9 having reduced nuclease activity comprises, or consists of, a sequence having at least 80, 85, 90, 95, 96, 97, 98, or at least 99% identity with a Cas9 wild-type protein such as described above and wherein the residue corresponding to D10, H840, R63, R66, R69, R70, R71, R74 and/or R78 of SEQ ID NO: 1 have been substituted, preferably with alanine. Preferably, the variant of Cas9 having reduced nuclease activity comprises, or consists of, a sequence having at least 80, 85, 90, 95, 96, 97, 98, or at least 99% identity with a Cas9 wild-type protein such as described above and wherein the residue corresponding to D10, H840, R63, R66, R69, R70, R71, R74 or R78 of SEQ ID NO: 1 have been substituted, preferably with alanine. The residue corresponding to D10, H840, R63, R66, R69, R70, R71, R74 or R78 of SEQ ID NO: 1 in a sequence of a Cas9 protein can be readily identified by conventional sequence alignment techniques.

In a preferred embodiment, the variant of Cas9 having reduced nuclease activity comprises, or consists of, a sequence having at least 80, 85, 90, 95, 96, 97, 98, or at least 99% identity with a Cas9 wild-type protein such as described above and wherein the residue corresponding to arginine at position 63, 66, 69, 70, 71, 74 and/or 78 of SEQ ID NO: 1 have been substituted, preferably with alanine. Preferably, the variant of Cas9 having reduced nuclease activity comprises, or consists of, a sequence having at least 80, 85, 90, 95, 96, 97, 98, or at least 99% identity with a Cas9 wild-type protein such as described above and wherein the residue corresponding to arginine at position 63, 66, 69, 70, 71, 74 or 78 of SEQ ID NO: 1 have been substituted, preferably with alanine.

Example of Cpf1 mutants exhibiting a reduced in vitro cleavage rate were disclosed in Swarts et al. (Swarts et al., 2017, Mol. Cell, 66, 221-233), such as Cpf1 mutant exhibiting Q704A mutation in SEQ ID NO: 17.

Thus, in another particular embodiment, the variant of Cpf1 having reduced nuclease activity comprises, or consists of, a sequence having at least 80, 85, 90, 95, 96, 97, 98, or at least 99% identity with a Cpf1 wild-type protein such as described above and wherein the residue corresponding to glutamine at position 704 of SEQ ID NO: 17 has been substituted, preferably with alanine.

In some other embodiments or additionally, the activity of the CRISPR nuclease is repressed during the mitotic phase by placing the expression of the CRISPR nuclease and/or guide RNA(s) under the control of a promoter inducible during the meiotic phase, preferably a promoter inducible during the meiotic prophase or a meiosis-specific promoter.

In particular, the nucleic acids encoding the CRISPR nuclease and/or the gRNA(s) may be placed under the control of a promoter inducible during the meiotic phase, preferably a promoter inducible during the meiotic prophase or a meiosis-specific promoter.

The nucleic acids encoding the CRISPR nuclease and/or those encoding the gRNAs may be placed under the control of different promoters. In embodiments wherein the cell comprises several guide RNAs, each guide RNA may be placed under the control of identical or different promoters, said promoter(s) being different from the promoter of the CRISPR nuclease.

In some embodiments, the nucleic acid encoding the CRISPR nuclease is placed under the control of a constitutive promoter and the nucleic acid(s) encoding the gRNA(s) is(are) placed under the control of a promoter inducible during the meiotic phase or a meiosis-specific promoter, or vice-versa.

Examples of suitable constitutive promoters for the CRISPR nuclease include, but are not limited to, ADH1 promoter, ubiquitin promoters, or GPM1 and TEF1 promoters. Examples of suitable constitutive promoters for the gRNA(s) include, but are not limited to the RNA polymerase III-dependent RPR1 and SNR52 promoters, and the polymerase III U3 and U6 promoters. Specifically in plants, the expression of CRISPR nuclease may be driven by Pol II promoters such as monocot or dicot specific ubiquitin promoters. In monocots, the maize ubiquitin promoter (ZmUbi3, SEQ ID NO 97) can be used for constitutive expression of CRISPR nuclease and for dicots the parsley ubiquitin or 2×35S promoters can be used (PcUbi 4-2, SEQ ID NO 98 and SEQ ID NO 99, respectively). The gRNA expression may be under the control of Pol III promoters such as U3 and U6 promoters. Arabidopsis promoters such as AtU6-26, (SEQ ID NO: 100) may be used in dicot plants, while rice promoters OsU3 and OsU6 (SEQ ID NO: 101 and 102, respectively) may be used for monocot plants. For meiosis-specific expression of CRISPR nuclease, Maize Spo11-1 promoter (ZmSPO11-1, SEQ ID NO: 103) can be used for monocots, and Tomato and Arabidopsis Spo11-1 promoters for dicots (SlSPO11-1 and AtSPO11-1, SEQ ID NO: 104 and 105, respectively). The nature of the promoter may depend on the nature of the eukaryotic cell and may be easily chosen by the skilled person.

In a particular embodiment, the nucleic acids encoding the CRISPR nuclease and/or the gRNA(s) are placed under the control of a promoter inducible during the meiotic phase. In particular, the induction of CRISPR nuclease and/or gRNA expression may be made when cells enter meiosis prophase I. Preferably, the nucleic acid encoding the CRISPR nuclease and the nucleic acid(s) encoding the gRNA(s) are placed under the control of a promoter inducible during the meiotic phase, in particular during the meiotic prophase. In particular, the inducible promoters may be different for the CRISPR nuclease and the gRNA(s)

Example of inducible systems and promoters suitable to be used in the present invention, in particular for the expression of the CRISPR nuclease or the fusion protein, are known in the art such as the estradiol promoter (Carlie & Amon, 2008, Cell, 133, 280-91), the methionine promoter (Care et al, 1999, Mol. Microbiology, 34, 792-798), the doxycycline-inducible TetO/TetR system, heat shock-induced promoters, metals, steroids, antibiotics and alcohol inducible promoter.

Preferably, the nucleic acids encoding the CRISPR nuclease and/or the gRNA(s) are placed under the control of a tetracycline operator, also known as the doxycycline-inducible TetO/TetR system. The expression of the CRISPR nuclease and/or the gRNA(s) may thus be regulated by the presence or absence of tetracycline or one of its derivatives such as anhydrotetracycline (ATc) doxycycline. The Tet system comprises two complementary circuits: the tTA dependent circuit (Tet-OFF system) and the rtTA dependent circuit (Tet-ON system). Such system is for example described in Smith et al. (Smith et al. 2016, Genome Biol., 17, 45). For example, the tetracycline repressor (TetR) and the CRISPR nuclease may be constitutively expressed whereas gRNA expression may be induced by addition of tetracycline, anhydrotetracycline (ATc) or doxycycline. Alternatively, the expression of CRISPR nuclease and gRNA(s) may be placed under the control of the tetracycline operator.

In another particular embodiment, the nucleic acids encoding the CRISPR nuclease and/or the gRNA(s) are placed under the control of a meiosis-specific promoter.

The nucleic acid encoding the CRISPR nuclease may be placed under the control of a meiosis-specific promoter and the nucleic acid(s) encoding the gRNA(s) may be placed under the control of a constitutive promoter or a promoter inducible during the meiotic phase, or vice-versa. Preferably, the nucleic acid encoding the CRISPR nuclease and the nucleic acid(s) encoding the gRNA(s) are placed under the control of a meiosis-specific promoter. In particular, the meiosis-specific promoters are different for the CRISPR nuclease and the gRNA(s).

Examples of meiosis-specific promoters suitable for use in the present invention, in particular for the expression of the CRISPR nuclease or the fusion protein, include, but are not limited to AtDMC1 promoter (in particular such as described in Xu et al., 2018, Front. Plant Sci. 9, 1-12), ZmDMC1 promoter (SEQ ID NO 23) and ZmSPO11-1 promoter (in particular such as described in WO2019224324), endogenous Spo11 promoters, promoters of Spo11's partners for double-strand break formation, the Rec8 promoter (Murakami & Nicolas, 2009, Mol. Cell. Biol, 29, 3500-3516), or the Spo13 promoter (Malkova et al, 1996, Genetics, 143, 741-754,), meiotic promoters from Arabidopsis thaliana for example such as described in Eid et al., (Eid et al., 2016, Plant Cell Rep., 35, 1555-1558), in particular a promoter specific to meiosis I, for example such as AT4G40020 also known as MGE1p, a promoter specific to meiosis I and II such as AT4G20900 also known as MGE2p, and a promoter specific to meiosis II such as AT1G15320 also known as MGE3p or other Arabidopsis thaliana meiosis-specific promoter such as described in Li et al.,2012, BMC Plant Biol., 12: 104, Eid et al, 2016, Plant Cell Rep.,35, 1555-1558, Xu et al, 2018, Front. Plant Sci, 9, 1007, Da Ines et al, 2013, PloS Genet., 9, e1003787.

Preferably, the meiosis specific promoter suitable for use in the present invention is selected from the group consisting of AtDMC1 promoter, Rec8 promoter, Spo13 promoter, MGE1p promoter, MGE2p promoter, MGE3p promoter, ZmDMC1 promoter, Maize Spo11-1 promoter (ZmSPO11-1), Tomato Spo11-1 promoter (SlSPO11-1) and Arabidopsis Spo11-1 promoter (AtSPO11-1), and variant thereof exhibiting at least 80%, 85%, 90%, 95%, 96%, 97%, 98%, or at least 99% identity to any of these promoters and a meiosis specific promoter activity.

More preferably, the meiosis specific promoter suitable for use in the present invention is selected from the group consisting of Maize Spo11-1 promoter (ZmSPO11-1, SEQ ID NO: 103) Tomato Spo11-1 promoter (SlSPO11-1, SEQ ID NO: 104) and Arabidopsis Spo11-1 promoter (AtSPO11-1, SEQ ID NO: 105), and variant thereof exhibiting at least 80%, 85%, 90%, 95%, 96%, 97%, 98%, or at least 99% identity to any of SEQ ID NO: 103, 104 or 105 and a meiosis specific promoter activity.

In some other embodiments, the activity of the CRISPR nuclease is repressed during the mitotic phase by using a system triggering the degradation of the CRISPR nuclease during the mitotic phase. Preferably, the system triggers the degradation of the CRISPR nuclease during the mitotic phase and not during the meiosis. In particular, the system may be inducible so that the CRISPR nuclease is degraded during the mitotic phase. Alternatively, the system may be inducible so that the CRISPR nuclease is not degraded during the meiotic phase, preferably during the prophase of meiosis. Alternatively or additionally, the system triggers the degradation of the gRNA(s) during the mitotic phase and preferably not during meiosis, especially during meiosis prophase. In a particular embodiment, the system triggering the degradation of the gRNA is a CIRTS biosensor platform based on guide RNA (gRNA)-dependent RNA binding domains that interact with a target transcript using Watson-Crick-Franklin base pair interactions. Such system is for example described in Rauch et al., 2020, ACS Cent. Sci., 6, 1987-1996.

In a particular embodiment, the system triggering the degradation of the CRISPR nuclease during the mitotic phase is an auxin-inducible degron system.

The auxin-inducible degron (AID) system uses a plant hormone-induced degradation signal to control protein levels and to conditionally target a protein of interest for ubiquitin-mediated proteolysis (Nishimura et al.,2014, Curr. Protoc. Cell Biol., 64:20.9.1-20.9.16, Natsume et al., 2016, Cell Rep. 2016,15, 210-218; Morawska & Ulrich, 2013, Yeast, 30, 341-351). Auxin (indole-3-acetic acid; IAA) induces degradation of a family of short-lived transcriptional repressors, the IAA proteins, by mediating the interaction of a degron domain in the target protein with the substrate recognition domain of an F-box protein, TIR1. Productive interaction in the presence of auxin leads to ubiquitylation of the target by recruitment of an SCF-type ubiquitin ligase (E3) and finally proteasomal degradation often in a matter of minutes in mitotically growing cells. The AID systems particularly contain two components: a degron, based on the IAA17 transcription repressor, that is fused to the protein of interest, and an SCF E3 ligase component, TIR1, usually from rice (OsTIR1). Auxin (usually indole acetic acid, IAA) promotes the interaction of the IAA17-based tag with TIR1, leading to the rapid polyubiquitylation of the target protein and its degradation by the proteasome.

Thus, the CRISPR nuclease may be fused to an auxin-dependent degron sequence derived from IAA17. This AID tag can be placed at the N- or C-terminus of the CRISPR nuclease. In particular, the AID-tag may comprise full-length IAA17, a 229 amino acid protein. Preferably, AID-tag comprises, or consists of, the sequence set forth in SEQ ID NO: 24.

An alternative approach to reversibly control gene expression is the use of ligand-dependent destabilization domains and the Shield- 1 ligand, which allows for reversible stabilization and destabilization of a tagged protein of interest such as a CRISPR nuclease in a dose-dependent manner (see, for example, Rakhit et al, Chemistry & Biology, 2014; 21: 1238-1252). Fusing the destabilizing domain to a gene of interest results in the expression a fused protein that is degraded by the proteasome. Shield-1 binds specifically to the destabilization domain and inactivates protein degradation.

Another approach includes inserting into the genome a nucleotide encoding a heterobifunctional compound targeting protein (dTAG) in-frame with the nucleotide sequence of a gene encoding an endogenously expressed protein of interest such as a CRISPR nuclease, which, upon expression, produces an endogenous protein-dTAG hybrid protein. This allows for targeted protein degradation of the dTAG and the fused endogenous protein using a heterobifunctional compound in a controlled, tunable fashion (see for example WO2017024319, incorporated herein by reference in its entirety).

In particular, to target the degradation of CRISPR nuclease in plants, a derivative of the AID system can be used. In the deGradFP system, the F-box protein from the SCF-type ubiquitin ligase (E3) is substituted with an engineered inducible form (Nslmb) fused to a nanobody (VhhGPF4) against a fluorescent reporter protein (GFP and its derivatives such as YFP, EYFP, etc.) that is fused to the protein of interest (see in particular Caussinus, et al., 2011, Nat. Struct. Mol. Biol., 19, 117-121, incorporated herein by reference). Thus, expression of the fusion protein NSlmb-VhhGPF4 leads to the nanobody-guided proteasomal degradation of the GFP-tagged proteins in plants (Sorge, et al., 2021, PLoS ONE, 16, e0247015). By tagging the CRISPR endonuclease with a fluorescent reporter protein and by controlling the expression of the NSlmb-VhhGPF4 fusion protein with an inducible promoter (such as an estradiol promoter). deGradFP could be used as an inducible system to trigger CRISPR endonuclease degradation in plants.

In some other embodiments, the activity of the CRISPR nuclease is repressed during the mitotic phase by using an inhibitor of the CRISPR nuclease.

The inhibitor of the CRISPR nuclease may be an antitoxin molecule, in particular an anti-CRISPR protein that inhibits the activity of the CRISPR nuclease. Preferably, said inhibitor is selected from the group consisting of AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2 and AcrIIC1, in particular such as disclosed in Table 1 of Pawluk, et al., 2018, Nat. Rev. Microbiol., 16, 12-17 (herein incorporated by reference).

Preferably, such inhibitor is expressed during the mitotic phase and is no longer expressed during meiosis, particularly during meiosis prophase. In particular, a nucleic acid encoding said inhibitor may be placed under the control of an inducible promoter, so that such inhibitor is expressed only outside of meiosis. Example of inducible promoters are known in the art such as the estradiol promoter (Carlie & Amon, 2008, Cell, 133, 280-91), the methionine promoter (Care et al, 1999, Mol. Microbiology, 34, 792-798), the doxycycline-inducible TetO/TetR system, heat shock-induced promoters, metals, steroids, antibiotics and alcohol inducible promoter.

In some other embodiments, the activity of the CRISPR nuclease is repressed during the mitotic phase by using a CRISPR nuclease which is a variant of a wild-type CRISPR nuclease, said variant exhibiting a reduced endonuclease activity by comparison to said wild-type CRISPR nuclease. Examples of such variants are provided herein, in particular “dead” nucleases such as dead-Cas9 or dead-Cpf1.

In some aspects, one or more of the above strategies to repress the activity of the CRISPR nuclease are combined in any of the methods disclosed herein, in particular to increase efficiency of the repression of nuclease activity during mitotic phase, particularly outside of meiosis.

In some embodiments, the activity of the CRISPR nuclease may be repressed during the mitotic phase i) by placing the expression of the CRISPR nuclease and/or the guide RNA(s) under the control of a promoter inducible during the meiotic phase or a meiosis-specific promoter and ii) by expressing an inhibitor of CRISPR endonuclease activity during the mitotic phase.

Preferably, the activity of the CRISPR nuclease may be repressed during the mitotic phase i) by placing the expression of the CRISPR nuclease under the control a meiosis-specific promoter and ii) by expressing an inhibitor of CRISPR endonuclease activity during the mitotic phase.

Alternatively, the activity of the CRISPR nuclease may be repressed during the mitotic phase i) by placing the expression of the guide RNA(s) under the control a meiosis-specific promoter and ii) by expressing an inhibitor of CRISPR endonuclease activity during the mitotic phase.

In some other embodiments, the activity of the CRISPR nuclease may be repressed during the mitotic phase i) by placing the expression of the CRISPR nuclease and/or the guide RNA(s) under the control of a promoter inducible during the meiotic phase or a meiosis-specific promoter and ii) by using a system triggering the degradation of the CRISPR nuclease and/or the guide RNA(s) during the mitotic phase.

Preferably, the activity of the CRISPR nuclease may be repressed during the mitotic phase i) by placing the expression of the CRISPR nuclease under the control a meiosis-specific promoter and ii) by using a system triggering the degradation of the CRISPR nuclease during the mitotic phase.

Alternatively, the activity of the CRISPR nuclease may be repressed during the mitotic phase i) by placing the expression of the guide RNA(s) under the control a meiosis-specific promoter and ii) by using a system triggering the degradation of the CRISPR nuclease during the mitotic phase.

In some other embodiments, the activity of the CRISPR nuclease may be repressed during the mitotic phase i) by placing the expression of the CRISPR nuclease and/or the guide RNA(s) under the control of a promoter inducible during the meiotic phase or a meiosis-specific promoter and ii) by using a CRISPR nuclease which is a variant of a wild-type CRISPR nuclease, said variant exhibiting a reduced endonuclease activity by comparison to said wild-type CRISPR nuclease.

In some other embodiments, the activity of the CRISPR nuclease may be repressed during the mitotic phase i) by expressing an inhibitor of CRISPR endonuclease activity during the mitotic phase and ii) by using a system triggering the degradation of the CRISPR nuclease during the mitotic phase.

In some other embodiments, the activity of the CRISPR nuclease may be repressed during the mitotic phase i) by expressing an inhibitor of CRISPR endonuclease activity during the mitotic phase and ii) by using a CRISPR nuclease which is a variant of a wild-type CRISPR nuclease, said variant exhibiting a reduced endonuclease activity by comparison to said wild-type CRISPR nuclease.

In some other embodiments, the activity of the CRISPR nuclease may be repressed during the mitotic phase i) by using a system triggering the degradation of the CRISPR nuclease during the mitotic phase and ii) by using a CRISPR nuclease which is a variant of a wild-type CRISPR nuclease, said variant exhibiting a reduced endonuclease activity by comparison to said wild-type CRISPR nuclease.

Any other combinations of the methods described herein to repress the activity of the CRISPR nuclease during the mitotic phase, are also contemplated.

In the method according to the invention, one or more gRNAs can be used simultaneously. These different gRNAs may target identical or different chromosomal regions.

Alternatively or in addition to the different techniques described above and used to repress the activity of the CRISPR nuclease during the mitotic phase, the method may further be designed to increase the probability of having at least one preserved site recognized by at least one guide RNA in the chromosomal region targeted during the meiotic phase by using a plurality of guide RNAs targeting the same chromosomal region.

Thus, in some embodiments, the gRNAs comprise a plurality of different gRNAs that target one particular chromosomal region of interest. The method may thus comprise the introduction into the cell of a plurality of gRNAs targeting the same chromosomal region, or a plurality of nucleic acids encoding said gRNAs. Preferably, the gRNAs comprise a plurality of guide RNAs targeting the same chromosomal region, or a plurality of nucleic acids encoding said guide RNAs, said chromosomal region being of less than 30 kilobase (kb) long.

In particular, the method may comprise introducing at least 2, 3, 4, 5, 6, 7, 8, 9, 10, 11, 12, 13, 14, 15 or more different gRNAs, preferably at least 3, 4, 5, 6, 7, 8, 9 or 10, even more preferably at least 5, 6, 7, 8, 9 or 10 different gRNAs targeting the same chromosomal region in the eukaryotic cell. Preferably, the methods described herein involve at least seven guide RNAs targeting the same chromosomal region. Preferably, the method comprises introducing from 5 to 20 (i.e., 5, 6, 7, 8, 9, 10, 11, 12, 13, 14, 15, 16, 17, 18, 19 or 20), from 5 to 15, from 5 to 10, from 7 to 20, from 7 to 15 or from 7 to 11 different guide RNAs targeting the same chromosomal region preferably between 3 and 15 (i.e., 3, 4, 5, 6, 7, 8, 9, 10, 11, 12, 13, 14, 15), or between 5 and 15, more preferably between 3 and 10 or between 5 and 10, particularly between 3 and 7 or 5 and 7 different gRNAs targeting the same chromosomal region in a eukaryotic cell. Particularly, the methods described herein involve between 5 and 20, between 7 and 20, between 7 and 15 or between 7 and 11 (i.e., 7, 8, 9, 10 or 11) different guide RNAs targeting the same chromosomal region.

Particularly, the methods described herein involve 5 or more guide RNAs targeting the same chromosomal region.

Preferably, the methods described herein involve 7 or more guide RNAs, in particular seven or more guide RNAs targeting the same chromosomal region.

Even more preferably, the methods described herein involve ten or more guide RNAs, in particular ten or more guide RNAs targeting the same chromosomal region.

By “different gRNAs targeting the same chromosomal region” it is meant herein that each of the gRNA targets a different nucleic acid sequence comprised in a particular chromosomal region. Preferably, the target chromosomal region is a region of 500 base pair (bp) to 20 000 bp, preferably between 500 and 10 000 bp, more preferably between 500 and 5000 bp, most preferably between 500 and 2500 bp. The same chromosomal region can be for example a promoter, a 3′ region of a gene of interest or any other regions of presumed open chromatin.

Preferably, the plurality of gRNA targets a given chromosomal region sequences each of the gRNA being spaced by at least 20, 40, 50, 60, 80, 100, 120, 150, 200, 500 or 1000 bp between one another. Particularly, the gRNAs are spaced from one another by between 50 bp and 1000 bp, preferably between 50 bp and 500 bp, more preferably between 50 and 250 bp, most preferably between 50 and 150 bp. More particularly, the gRNAs may be regularly spaced by 146 bp, i.e. the length of DNA wrapping around a nucleosome.

These guide RNAs are different in terms of sequences but may share overlapping regions/sequences between each other. Preferably, the guide RNAs differ in terms of sequence identity by at least 2%, 5%, 10%, 15%, 20%, 25%, 30%, 35%, 40%, 45%, 50%, 55%, 60%, 65%, 75%, 80%, 85%, 90%, 95% or 100%.

Each gRNA of the plurality of gRNAs may have the same or different lengths, preferably of the same length.

In a particular embodiment, the method may comprise introducing between 3 and 15 gRNAs, preferably between 3 and 7 gRNAs, targeting the same chromosomal region, said chromosomal region being of between 1000 and 2500 bp and the gRNAs being spaced by between 100 and 200 bp.

In another embodiment, the method may comprise introducing between 5 and 15 gRNAs, preferably between targeting the same chromosomal region, said chromosomal region being of between 2.5 kb and 20 kb and the gRNAs being spaced by between 250 and 1000 bp.

In another particular embodiment, the method may comprise introducing between 3 and 15 different gRNAs, particularly between 3 and 7 different gRNAs, targeting the same chromosomal region, said chromosomal region being a pericentromeric region.

The method of the invention may further comprise introducing into the cell a fusion protein comprising a Spo11 protein operably linked to a DNA binding domain, or a nucleic acid encoding said fusion protein.

Then, the invention also concerns a method for inducing targeted meiotic recombination in a cell, said method comprising:
introducing into said cell:
a) a CRISPR nuclease, preferably a class II CRISPR nuclease or a nucleic acid encoding said nuclease,
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region; and
c) a fusion protein comprising a Spo11 protein or one of the Spo11 partners, operably linked to a DNA binding domain, or a nucleic acid encoding said fusion protein, preferably under the control of a meiosis-specific promoter; and
inducing said cell to enter meiotic prophase I,
wherein the CRISPR nuclease is not fused with a Spo11 protein, optionally wherein the activity of the CRISPR nuclease is repressed outside of meiosis, and
wherein the cell is preferably a yeast cell, a plant cell or a fungus cell.

Preferably, the activity of the CRISPR nuclease is repressed outside of meiosis.

Preferably, the CRISPR nuclease provided under a) is not fused with:
a Spo11 protein or a variant or fragment thereof as defined herein; and/or
a Spo11 partner as defined herein, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; and/or
a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; and/or
a DNA binding domain; and/or
a protein/amino acid sequence comprising at least 50 amino acids.

This fusion protein may be placed under the control of a constitutive, inducible or meiosis-specific promoter, preferably under the control of a meiosis-specific promoter.

As used herein, the term “fusion protein” refers to a chimeric protein comprising at least two domains derived from the combination of different proteins or protein fragments. The nucleic acid encoding this protein may be obtained by recombination of the regions encoding the proteins or protein fragments so that they are in phase and transcribed on the same mRNA. The various domains of the fusion protein may be directly adjacent or may be separated by an amino acid sequence (linker) which introduce a certain structural flexibility into the construction.

The fusion protein used in the present invention comprises (i) a Spo11 protein or one of the Spo11 partner and (ii) a DNA binding domain. Preferably, the fusion protein used in the present invention comprises a Spo11 protein and a DNA binding domain.

Spo11 is a protein related to the catalytic A subunit of a type II topoisomerase present in archaebacteria (Bergerat et al., 1997, Nature, 386, 414-417). It catalyzes the DNA double-strand breaks initiating meiotic recombination. It is a highly conserved protein for which homologs exist in all eukaryotes. Spo11 is active as a dimer formed of two subunits, each of which cleaves a DNA strand. Although essential, Spo11 does not act alone to generate double-strand breaks during meiosis. In the yeast S. cerevisiae, for example, it cooperates with Rec102, Rec103/Sk18, Rec104, Rec114, Mer1, Mer2/Rec107, Mei4, Mre2/Nam8, Mre11, Rad50, Xrs2/Nbs1, Hop1, Red1, Mek1, Seti and Spp1 proteins and with other partners described in the articles by Keeney et al., 2001, Curr. Top. Dev. Biol, 52. 1-53, Smith et al. 1998, Curr. Opin. Genet. Dev., 8, 200-211) and Acquaviva et al., 2013, Science, 339, 215-218). It should be noted that several Spo11-related protein homologs can coexist in the same cell, notably in plants. Preferably, the Spo11 protein is one of the Spo11 proteins of the eukaryotic cell of meiotic interest.

The Spo11 protein may be obtained from any known Spo11 proteins. Examples of Spo11 proteins include, but are not limited to, the Spo11 protein from Saccharomyces cerevisiae (Gene ID: 856364, NCBI entry number: NP_011841; Esposito & Esposito, 1969, Genetics, 61, 79-89; SEQ ID NO: 25), Arabidopsis thaliana (e.g. Uniprot accession number: Q9M4A2-1; SEQ ID NO: 27), Oryza sativa (rice) (e.g. as described by Fayos I. et al., 2019 Plant Biotechnol J. 17, 2062-2077 and under Uniprot accession numbers: Q2QM00 (SEQ ID NO: 28), Q7Y021 (SEQ ID NO: 29), Q5ZPV8 (SEQ ID NO: 30), A2XFC1 (SEQ ID NO: 31) and Q6ZD95(SEQ ID NO: 32), Brassica campestris (mustard) (e.g UniProt accession numbers: A0A024AGF2 (SEQ ID NO: 33) and A0A024AHI2 (SEQ ID NO: 34), Zea mays (corn) (e.g. Uniprot accession numbers: B6UAQ8 (SEQ ID NO: 35) and B6TWI5 (SEQ ID NO: 36), A0A1P8W169-1 (SEQ ID NO: 37) and A0A1P8W163 (SEQ ID NO: 38)), Capsicum baccatum (pepper tree) (e.g Uniprot accession numbers: A0A2G2WFG5 (SEQ ID NO: 39) and A0A2G2WFH4 (SEQ ID NO: 40), Carica papaya (papaya) (e.g. Uniprot accession number: A0A024AG98 (SEQ ID NO: 41), and Solanum lycopersicum (tomato, NCBI Reference Sequence: XP_010324270.1, SEQ ID NO: 42).

Preferably, the Spo11 protein comprised in the fusion protein is from Oryza sativa, Brassica campestris, Zea mays, Capsicum baccatum, Carica papaya or Solanum lycopersicum.

Preferably, the Spo11 protein is a Spo11-1 or a Spo11-2 protein. By way of example, a fusion protein according to the invention may comprise Arabidopsis thaliana Spo11-1 and/or Spo11-2 domains. Also by way of example, one or more fusion proteins according to the invention comprising rice Spo11-1, Spo11-2 and/or Spo11-3 domain(s). Also by way of example, one or more fusion proteins according to the invention comprising tomato Spo11-1, Spo11-2 and/or Spo11-3 domain(s).

Preferably, the Spo11 protein of the fusion protein comprises, or consists of, a Spo11 protein, preferably a wild-type Spo11 protein, in particular a Spo11 protein of the eukaryotic cell of interest, or a sequence having at least 80%, 85%, 90%, 95%, 96%, 97%, 98%, or at least 99% identity with said Spo11 protein and exhibiting a Spo11 activity.

In particular, the Spo11 protein may comprise, or consist of, an amino acid sequence selected from the group consisting of SEQ ID NO: 25 and 27 to 42 and variants or functional fragments thereof exhibiting Spo11 activity and having at least 70%, preferably at least 80%, more preferably at least 90%, 95%, 96%, 97%, 98% or 99%, sequence identity to any of SEQ ID NO: 25 and 27 to 42.

As used herein, the term “Spo11 activity” refers to the ability of a protein to induce double-strand breaks during prophase I of meiosis and/or the ability of a protein to recruit one or more Spo11 partners as defined below. Preferably, this term refers to the ability of a protein to recruit one or more partners of Spo11 and, optionally, the ability of a protein to induce double-strand breaks during prophase I of meiosis. The ability of a protein to induce double-strand breaks during prophase I of meiosis and to recruit one or more Spo11 partners can be easily tested by the skilled person in the art, for example by a complementation assay in yeast or in a plant in which endogenous Spo11 protein has been inactivated. If the protein has Spo11 activity, that organism will produce viable spores. The ability of one protein to recruit another, for example a protein to recruit a partner of Spo11 or a partner of Spo11 to recruit a Spo11 protein, can be readily tested by those skilled in the art using conventional techniques, such as the double hydride technique or the ChIP (chromatin immunoprecipitation) technique. The ability of a protein to induce double-strand breaks during prophase I of meiosis can be easily tested by the person skilled in the art using conventional techniques such as Southern blot or sequencing of oligonucleotides associated with a protein, in particular Spoil.

The Spo11 protein may exhibit nuclease activity or may be a variant exhibiting deficient nuclease activity. For a wild-type Spo11 protein or a variant with nuclease activity, the term “Spo11 activity” preferably refers to the ability of a protein to induce double-strand breaks during prophase I of meiosis and the ability of a protein to recruit, directly or indirectly, one or more Spo11 partners as defined below. For a Spo11 variant that exhibits deficient nuclease activity, the term “Spo11 activity” preferably refers to the ability of a protein to recruit one or more Spo11 partners as defined below. A Spo11 variant exhibiting deficient nuclease activity may comprise, or consist of, the Spo11-Y135F mutant protein, a mutant protein incapable of inducing DNA double-strand breaks (Bergerat et al. 1997, Nature, 386, 414-417). The position indicated is that of SEQ ID NO:25. Such mutant protein is for example described under the sequence set forth in SEQ ID NO: 26. Thus, the Spo11 variant may comprise, or consist of, a Spo11 protein, preferably selected from the group consisting of SEQ ID NO: 25 and 27 to 42, in particular a Spo11 protein of the eukaryotic cell of interest, or a sequence having at least 80%, 85%, 90%, 95%, 96%, 97%, 98%, or at least 99% identity with said Spo11 protein and wherein the residue corresponding to the tyrosine at position 135 of SEQ ID NO: 25 has been substituted, preferably with phenylalanine.

In preferred embodiments, the Spo11 protein of the fusion protein exhibits nuclease activity.

As used herein, the term “Spo11 fragment” refers to a fragment of a Spo11, comprising at least 100, 150, 200, 250, 300, 350, 400, 450 or 500 contiguous amino acids of said Spo11 protein, and retaining the activity of the entire polypeptide, i.e., exhibiting nuclease activity.

Alternatively, the Spo11 protein of the fusion protein can be replaced by one of the Spo11 partners involved in the formation and repair of double-strand breaks during meiosis. In particular, the partner of Spo11 as used in the fusion protein is capable of recruiting Spoil, preferably is a protein that forms a complex with Spo11 and thereby induces double-strand break formation or repair. This partner can be selected from the proteins cited in the articles by Keeney et al., 2001, Curr. Top. Dev. Biol, 52, 1-53), Smith et al., 1998, Curr. Opin. Genet. Dev, 8, 200-211, Acquaviva et al., 2013, Science, 339, 215-218, Vrielynck et al., 2016, Science, 351, 939-943), Roberts et al., 2016, Science, 351, 943-949 and Blattner, 2016, Plant Syst. Evol. 302, 239-244. Preferably, the fusion protein according to the invention comprises a partner of Spo11 selected from Rec102, Rec103/Sk18, Rec104, Rec114, MTOPOVIB, TOPOVIB, Mer1, Mer2/Rec107, Mei4, Mre2/Nam8, Mre11, Rad50, Xrs2/Nbs1, Hop1, Red1, Mek1, Set1, Ski8, and Spp1, and variants and orthologs thereof. Preferably, the partner of Spo11 comprises a protein selected from Mei4, Mer2, Rec102, Rec104, Rec114, Set1, Spp1, and MTOPVIB, and variants and orthologues thereof. As contemplated herein, variants of these proteins have at least 80, 85, 90, 95, 96, 97, 98, or at least 99% sequence identity to any of these proteins and are capable of recruiting Spoil.

The fusion protein also comprises a DNA binding domain (DBD) fused to the Spo11 protein (or the Spo11 partner). A DBD can recognize a specific DNA sequence (a recognition sequence) or have a general affinity to DNA. In particular, the DNA binding domain may be selected from the group consisting of TAL effector DNA binding domains, B3 DNA binding domains, zinc finger DNA binding domains, helix-turn-helix DNA binding domains, leucine zipper DNA binding domains, HMG-box domains, transcription factor DNA binding domains such as GAL4 binding domain, inactivated CRISPR nucleases and inactivated meganucleases.

The DNA binding domain may be a TAL effector DNA binding domain. TAL effectors are found in bacterial plant pathogens of the genus Xanthomonas. They contain a central region of tandem 33-35 residue repeats and each repeat region encodes a single DNA base in the TALE's binding site. Residue 13 alone directly contacts the DNA base, determining sequence specificity, while other positions make contacts with the DNA backbone, stabilizing the DNA-binding interaction. Related proteins are found in bacterial plant pathogen Ralstonia solanacearum or in the fungal endosymbiont Burkholderia rhizoxinica.

The DNA binding domain may be a B3 DNA binding domain. The B3 DBD (InterPro: IPR003340, SCOP 117343) is found exclusively in transcription factors from higher plants and restriction endonucleases EcoRII and BfiI and typically consists of 100-120 residues. It includes seven beta sheets and two alpha helices, which form a DNA-binding pseudobarrel protein fold.

The DNA binding domain may be a zinc finger DNA binding domain. The zinc finger domain is generally between 23 and 28 amino acids long and is stabilized by coordinating zinc ions with regularly spaced zinc-coordinating residues (either histidine or cysteine). The most common class of zinc finger (Cys2His2) coordinates a single zinc ion and consists of a recognition helix and a 2-strand beta-sheet.

The DNA binding domain may be a helix-turn-helix DNA binding domain. The helix-turn-helix motif is commonly found in repressor proteins and is about 20 amino acids long. In eukaryotes, the homeodomain comprises 2 helices, one of which recognizes the DNA (aka recognition helix).

The DNA binding domain may be a leucine zipper DNA binding domain. The bZIP domain contains an alpha helix with a leucine at every 7th amino acid. If two such helices find one another, the leucine can interact as the teeth in a zipper, allowing dimerization of two proteins.

When binding to the DNA, basic amino acid residues bind to the sugar-phosphate backbone while the helices sit in the major grooves.

The DNA binding domain may be a HMG-box domain. The domain consists of three alpha helices separated by loops.

The DNA binding domain may be a transcription factor DNA binding domain such as a Gal4-binding domain (Gal4BD). This domain is a zinc finger and belongs to the Zn(2)-C6 fungal family. It has recently been shown that it is possible to modify double-strand break formation sites by fusing Spo11 to the DNA-binding domain of the transcriptional activator Gal4 (Pecina et al, 2002, Cell, 111, 173-184). In a particular embodiment, the fusion protein comprises a Spo11 partner, in particular such as Rec102, Rec104, Rec 114, MER2 or MEI4, and a Gal4BD, for example such as described in Koehn et al., 2009, Genetics, 182, 447-458.

The DNA binding domain may be an inactivated CRISPR nuclease, in particular a dead Cas9 (dCas9) or dead Cpf1 (dCpf1) or an inactivated meganuclease.

By “dead” CRISPR nuclease it is meant a nuclease that has a nuclease activity that is reduced or abolished but that still retains its ability to bind DNA in a sgRNA-dependent manner. In some cases, the nuclease activity of a “dead” CRISPR nuclease is reduced but not completely abolished, in particular in comparison with the nuclease activity of a wild type nuclease.

Cas9 proteins comprise two nuclease domains: a domain related to a RuvC domain and a domain related to an HNH domain. These two domains cooperate to create DNA double-strand breaks (Jinek et al., 2012, Science, 337: 816-821). Each of these nuclease domains can be inactivated by deletion, insertion or substitution according to techniques well-known to a person skilled in the art such as directed mutagenesis, PCR mutagenesis or total gene synthesis.

In particular, a dCas9 may be a variant of a Cas9 wild-type protein having a deficient nuclease activity and comprising a sequence having at least 80, 85, 90, 95, 96, 97, 98, or at least 99% identity with a Cas9 wild-type protein such as described above and wherein the residue corresponding to aspartate at position 10 of SEQ ID NO: 1 and the residue corresponding to the histidine at position 840 of SEQ ID NO: 1 have been substituted, preferably with alanine.

A variant of a Cpf1 nuclease deficient in nuclease activity may comprise, or consist of, a mutant Cpf1 protein for example as described in Zhang et al., 2018, Cell Discov., 4, 36, having the mutation D832A (position corresponding to SEQ ID NO:16) and corresponding to the dead LbCpf1 protein (SEQ ID NO: 19). A Cpf1 protein with such a substitution is unable to induce DNA double-strand breaks, and in particular may take the name “dead Cpf1” or “dCpf1”. A dCpf1 may be a variant of a Cpf1 wild-type protein having a deficient nuclease activity and comprising a sequence having at least 80, 85, 90, 95, 96, 97, 98, or at least 99% identity with a Cpf1 wild-type protein such as described above and wherein the residue corresponding to the aspartate at position 917 of SEQ ID NO: 17 is substituted, preferably by an alanine (dead FnCpf1, SEQ ID NO: 18). A variant of a Cpf1 nuclease deficient in nuclease activity may comprise, or consist of a sequence as set forth in SEQ ID NO: 18 or SEQ ID NO: 19 or a sequence having at least 80, 85, 90, 95, 96, 97, 98, or at least 99% identity thereto.

The Spo11 domain may be on the N-terminal side and DBD domain may be on the C-terminal side of the fusion protein, or vice-versa.

The fusion protein may also comprise a nuclear localization signal (NLS) sequence, a cell-penetrating domain, i.e., a domain facilitating the entry of the fusion protein into the cell, and/or a tag.

The fusion protein may further comprise one or more amino acid sequences (linkers) between the DBD and the Spo11 protein, and optionally between these domains and the other domains of the protein such as the nuclear localization signal sequence or the cell-penetrating domain. The length of these linkers is readily adjustable by a person skilled in the art. In general, these sequences comprise between 10 and 20 amino acids, preferably about 15 amino acids and more preferably 12 amino acids.

The method of the invention comprises inducing the cell to enter meiotic prophase I.

This induction can be done according to various methods, well known to the man of the art.

For example, when the eukaryotic cell is a mouse cell, the entry of the cells into prophase I of meiosis can be induced by the addition of retinoic acid (Bowles et al, 2006, Sciences, 312, 596-600).

When the eukaryotic cell is a plant cell, the induction of meiosis occurs by a natural process.

In particular, after transformation of a callus comprising one or more plant cells, a plant may be regenerated and placed in conditions favoring the induction of a reproductive phase and thus of the meiosis process. These conditions are well known to the skilled person.

When the eukaryotic cell is a yeast, this induction can be achieved by transferring the yeast into a sporulation medium, in particular from a rich medium to a sporulation medium, said sporulation medium preferably being devoid of a fermentable carbon or nitrogen source, and incubating the yeast in the sporulation medium for a sufficient time to induce double-strand breaks. Initiation of the meiotic cycle depends on several signals: the presence of the two sex-type alleles MATa and MATα, the absence of a source of nitrogen and fermentable carbon.

The method may further comprise obtaining the cell or cells having the desired recombination(s). When the cell is a yeast cell, the process may further comprise a step of culturing and/or multiplying the cell or cells having the desired recombination(s). When the cell is a plant cell, the process may further comprise a somatic embryogenesis step, i.e. the regeneration of a plant embryo from a callus comprising the cells having the desired recombination(s).

In another aspect, the present invention relates to a method for generating variants of a non-human eukaryotic organism, said method comprising:
introducing into a cell of said organism:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell.

Preferably, the CRISPR nuclease is not fused with a Spo11 protein.

Particularly, the present invention relates to a method for generating variants of an organism, said method comprising:
introducing into a cell of said organism:
a) a CRISPR nuclease, preferably a class II CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the activity of the CRISPR nuclease is repressed outside of meiosis,
wherein the CRISPR nuclease is not fused with a Spo11 protein, and
wherein the organism is a yeast, a plant or a fungus.

Preferably, the present invention relates to a method for generating variants of an organism, said method comprising:
introducing into a cell of said organism:
a) a CRISPR nuclease, preferably a class II CRISPR nuclease or a nucleic acid encoding said nuclease, and
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the CRISPR nuclease is not fused with a Spo11 protein and wherein the activity of the CRISPR nuclease is repressed outside of meiosis, and
wherein the organism is a yeast, a plant or a fungus.

Preferably, the CRISPR nuclease provided under a) is not fused with:
a Spo11 protein or a variant or fragment thereof such as defined herein; and/or
a Spo11 partner such as defined herein, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; and/or
a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; and/or
a DNA binding domain in particular such as defined herein; and/or
a protein/amino acid sequence comprising at least 50 amino acids.

In an embodiment, the activity of the CRISPR nuclease is repressed during the mitotic phase as described above.

In another embodiment, the method comprises introducing into said cell a plurality of guide RNAs targeting the same chromosomal region or a plurality of nucleic acids encoding said guide RNAs.

In a further embodiment, the activity of the CRISPR nuclease is repressed during the mitotic phase and the method comprises introducing into said cell a plurality of guide RNAs targeting the same chromosomal region or a plurality of nucleic acids encoding said guide RNAs.

All embodiments described above for the method of the invention for inducing targeted meiotic recombination are also contemplated in this aspect.

CRISPR nuclease, gRNA, Spo11 fusion protein or CRISPR nuclease inhibitor and nucleic acids encoding thereof are heterologous to the host cell. The term “heterologous”, with respect to a host cell, refers to a genetic element or a protein that is not naturally present in said host cell. As used herein, the term “native” or “endogenous”, with respect to a host cell, refers to a genetic element or a protein naturally present in said host cell.

Preferably, a heterologous nucleic acid is comprised in an expression cassette. In embodiments wherein several heterologous nucleic acid are introduced in the host cell, these heterologous nucleic acids may be comprised in one or several expression cassettes.

In the context of the invention, by “nucleic acid” is meant any molecule based on DNA or RNA. These molecules may be synthetic or semisynthetic, recombinant, optionally amplified or cloned into vectors, chemically modified, comprising non-natural bases or modified nucleotides comprising for example a modified bond, a modified purine or pyrimidine base, or a modified sugar. Preferably, the use of codons is optimized according to the nature of the host cell.

The term “expression cassette” denotes a nucleic acid construct comprising a coding region, e.g. one or several genes or coding sequences, and a regulatory region, i.e. a region comprising one or more control sequences, operably linked. Optionally, the expression cassette may comprise several coding regions operably linked to several regulatory regions. In particular, the expression cassette may comprise several coding sequences, each of these sequences being operably linked to the same promoter. Alternatively, the expression cassette may comprise one or several coding sequences, each of these sequences being operably linked to a distinct promoter. The expression cassette may also comprise one or several coding sequences, each of these sequences being operably linked to a distinct promoter and one or several other coding sequences being operably linked to a common promoter.

The term “operably linked” means a configuration in which a control sequence is placed at an appropriate position relative to a coding region, in such a way that the control sequence directs expression of the coding region.

The term “control sequence” means a nucleic acid sequence necessary for expression of a coding region. Control sequences may be native or heterologous. Well-known control sequences and currently used by the person skilled in the art will be preferred. Such control sequences include, but are not limited to, a leader, polyadenylation sequence, propeptide sequence, promoter, signal peptide sequence, and transcription terminator. Preferably, the control sequences include a promoter and a transcription terminator. In particular the promoter(s) used in the expression cassette may be any constitutive, inducible or meiosis-specific promoter as described above.

An expression cassette introduced in the host cell may be integrated into the genome of the cell and/or may be maintained in an episomal form into an expression vector. Preferably, the expression cassette(s) is(are) integrated into the genome of the cell.

In a further aspect, the present invention relates to an expression cassette comprising one or more heterologous nucleic acids as described above. In particular, the expression cassette may comprise a nucleic acid encoding for (i) a CRISPR nuclease as described above, (ii) one or several guide RNAs, (iii) a fusion protein comprising a Spo11 protein, (iv) a CRISPR nuclease inhibitor, and any combination thereof. In particular the promoter(s) used in the expression cassette may be any constitutive, inducible or meiosis-specific promoter as described above.

The present invention further relates to an expression vector comprising a heterologous nucleic acid or an expression cassette as described above.

Said expression vector may be used to transform a host cell and enable the expression of the heterologous nucleic acid in said cell. The choice of the vector will typically depend on the compatibility of the vector with the host cell into which the vector is to be introduced. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication and maintenance. Alternatively, the vector may be one that, when introduced into the host cell, is integrated into the genome and replicated together with the chromosome(s) into which it has been integrated.

The vector preferably comprises one or more selectable markers that permit easy selection of host cells comprising the vector. A selectable marker is a gene the product of which provides for biocide or viral resistance, resistance to heavy metals, prototrophy to auxotrophy, and the like.

The vector preferably comprises an element that permits integration of the vector into the host cell's genome or autonomous replication of the vector in the cell independent of the genome. When integration into the host cell genome occurs, integration of the sequences into the genome may rely on homologous or non-homologous recombination. In one hand, the vector may contain additional polynucleotides for directing integration by homologous recombination at a precise location into the genome of the host cell. These additional polynucleotides may be any sequence that is homologous with the target sequence in the genome of the host cell. On the other hand, the vector may be integrated into the genome of the host cell by non-homologous recombination.

The expression vector may comprise one or more bacterial or eukaryotic origins of replication. The expression vector may in particular include a bacterial origin of replication functional in E. coli such as the ColE1 origin of replication. Alternatively or additionally, the vector may comprise a eukaryotic origin of replication, preferably functional in plant or in yeast, in particular such as S. cerevisiae.

The methods for selecting these elements according to the host cell in which expression is desired, are well known to one of skill in the art. The vectors may be constructed by classical techniques of molecular biology, well known to one of skill in the art.

The present invention also relates to the use of an expression cassette or an expression vector according to the invention to transform or transfect a cell. The present invention also relates to a host cell comprising an expression cassette or an expression vector according to the invention.

The host cell may be transformed/transfected in a transient or stable manner and the cassette or the vector may be contained in the cell as an episome or integrated into the genome of the host cell. The term “host cell” also encompasses any progeny of a parent host cell that is not identical to the parent host cell due to mutations that occur during replication.

The expression cassette or expression vector according to the invention may be introduced into the host cell by any method known by the skilled person, such as electroporation, conjugation, transduction, competent cell transformation, protoplast transformation, protoplast fusion, biolistic device or gene gun based transformation, PEG-mediated transformation, lipid-assisted transformation or transfection, chemically mediated transfection, lithium acetate-mediated transformation and liposome-mediated transformation.

More specifically, plant transformation techniques are well known and described in the technical and scientific literature. These techniques aim at the transformation of plant cells from whole plants, callus or protoplasts. These techniques include injection or microinjection (Griesbach, 1987, Plant Sci. 50, 69-77), DNA electroporation (Fromm et al., 1985, Proc. Natl. Acad. Sci. USA, 82: 5824-5828; Wan & Lemaux, 1994, Plant Physiol. 104, 37-48), biolistics (Klein et al., 1987, Nature, 327: 70-73), viral vector transfection (Gelvin, 2005, Nat. Biotechnol., 23, 684-685, bombardment (Sood et al, 2011, Biologia Plantarum, 55, 1-15), cell or protoplast fusion (Willmitzer, 1993, Transgenic plants in: Biotechnology, vol. 2, 627-659), agrotransfection by T-DNA insertion, including using Agrobacterium tumefaciens (Fraley et al. Crit. Rev. Plant. Sci., 4, 1-46; Fromm et al., 1990, Nat. Biotechnol., 8, 833-839) or Agrobacterium rhizogenes (Cho et al., 2000, Planta, 210, 195-204) or other bacterial hosts (Brootghaerts et al., 2005, Nature, 433, 629-633) using, for example, the so-called floral dip technique (Clough & Bent, 1998 The Plant Journal, 16, 735-743; Zale et al., 2009, Plant Cell Rep., 28, 903-913).

Of note, CRISPR/Cas9 based genome editing in plants has mostly been achieved using Agrobacterium tumefaciens based methods. However, various viral vectors, such as geminiviruses, tobamoviruses, potexviruses, tobravirus and comoviruses have been successfully used to deliver the gene editing machinery into plants particularly dicots.

The use of viruses to deliver components of CRISPR/Cas in plants was first reported in 2014 using geminiviruses (Baltes et al, 2014, Plant Cell, 26, 151-163). Since then, both RNA based viruses (Ali et al., 2016, Sci. Rep., 6, 26912; Ali et al., 2015, Genome Biol., 16; Ali et al., 2018, Virus Res., 244, 333-337; Cody et al, 2017, Plant Physiol., 175, 23-35; Hu et al., 2019, Mol. Plant Pathol., 20, 1463, 1474; Mei et al., 2019, Plant Direct, 3, e00181) and DNA based viruses (Wang et al., 2017, Mol. Plant., 10, 1007-1010; Gil-Humanes et al., 2017, Plant J.,89, 1251-1262; Yin et al., 2015, Sci. Rep., 5, 14926; Cermak et al., 2015, Genome Biol., 16, 232) have been used for the successful delivery of gene editing machinery into the plant cells. While most of these virus-based methods are limited to dicot plants, geminiviruses have been used in both monocot (for example Barley stripe mosaic virus) and dicot plants.

Geminiviruses are small (˜3 kb), diverse, single strand DNA viruses that infect a wide variety of plants including many important crop species. In nature, Geminiviruses are transmitted to host plants through insects. The easy manipulation of geminiviruses due to their small size is advantageous for genome editing techniques, but rather limits the size of DNA fragments they can carry to host cells. To increase their capacity to carry larger DNA fragments, these viruses have been modified into non-infectious replicons. These modified viruses are unable to infect the plant cells and are delivered to plant cells using Agrobacterium. This Agrobacterium based method of geminivirus delivery has been successfully used for CRISPR/Cas gene editing in various crops such as tobacco (Baltes et al, 2014, Plant Cell, 26, 151-163), potato (Butler et al., 2016, Front. Plant Sci., 7, 1045), tomato (Cermak et al., 2015, Genome Biol., 16, 232), rice (Wang et al., 2017, Mol. Plant., 10, 1007-1010), maize (Mei et al., 2019, Plant Direct, 3, e00181). Studied using this method have reported much higher genome targeting efficiency, when compared to the traditional Agrobacterium based T-DNA insertion methods (Baltes et al, 2014, Plant Cell, 26, 151-163).

Thus, in an embodiment, wherein the cell is a plant cell, delivery of nucleic acid encoding CRISPR nuclease and/or sgRNA components and/or fusion protein and/or any other elements described above, is achieved by using viral vectors, such as geminiviruses, tobamoviruses, potexviruses, tobravirus or comoviruses, by in planta particle bombardment, for example using shoot apical meristems of mature seeds or by biolistic DNA delivery without callus culture. In particular, preassembled CRISPR nuclease-sgRNA ribonucleoproteins can be delivered into plant shoot apical meristems to generate gene edits or to introduce edits into pollen and inflorescence tissues. Preferably, in the methods of the invention, when the cell is a plant cell, the CRISPR nuclease and/or the guide RNA(s) are introduced into the plant cell, preferably into inflorescence, by a viral vector, preferably a viral vector derived from a Geminivirus.

Alternatively, and more particularly with respect to plant cells, the heterologous nucleic acid(s), expression cassette(s) or expression vector(s) may be introduced into a host cell by crossing two cells into which the heterologous nucleic acid(s), expression cassette(s) or expression vector(s) have been introduced.

Optionally, more than one copy of an expression cassette or vector of the present invention may be inserted into the host cell.

The present invention also concerns the use of an expression cassette or vector of the present invention according to the invention to (i) induce targeted meiotic recombination in a eukaryotic cell, (ii) generate variants of a eukaryotic organism, preferably according to the methods of the invention. All embodiments described above for the methods, expression cassette and vector of the invention are also contemplated in this aspect.

In another aspect, the present invention also relates to a eukaryotic host cell, in particular a non-human eukaryotic host cell, comprising:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region, and
wherein the activity of the CRISPR nuclease is repressed during the mitotic phase by
(i) placing the expression of said CRISPR nuclease and/or said guide RNAs under the control of a promoter inducible during the meiotic phase or a meiosis-specific promoter, and/or
(ii) expressing an inhibitor of CRISPR endonuclease activity during the mitotic phase, and/or
(iii) using a system triggering the degradation of the CRISPR nuclease during the mitotic phase, and/or
(iv) using CRISPR nuclease which is a variant of a wild-type CRISPR nuclease, said variant exhibiting a reduced endonuclease activity by comparison to said wild-type CRISPR nuclease.

All embodiments described above for the method of the invention for inducing targeted meiotic recombination are also contemplated in this aspect.

The invention also concerns a host cell comprising:
a) a CRISPR nuclease, preferably a class II CRISPR nuclease, or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region, and
c) optionally a fusion protein comprising a Spo11 protein operably linked to a DNA binding domain, or a nucleic acid encoding said fusion protein,
wherein the CRISPR nuclease is not fused with a Spo11 protein and
wherein the activity of the CRISPR nuclease is repressed outside of meiosis by
(i) placing the expression of said CRISPR nuclease and/or said guide RNAs under the control of a promoter inducible during the meiotic phase or a meiosis-specific promoter, and/or
(ii) expressing an inhibitor of CRISPR endonuclease activity outside of meiosis, and/or
(iii) using a system triggering the degradation of the CRISPR nuclease outside of meiosis, and/or
(iv) using a CRISPR nuclease which is a variant of a wild-type CRISPR nuclease, said variant exhibiting a reduced endonuclease activity by comparison to said wild-type CRISPR nuclease;
wherein the host cell is a yeast cell, a plant cell or a fungus cell.

All embodiments described above for the method of the invention for inducing targeted meiotic recombination are also contemplated in this aspect.

The host cell may comprise (i) a nucleic acid encoding a CRISPR nuclease and/or a nucleic acid encoding a guide RNA placed under the control of a promoter inducible during the meiotic phase or a meiosis-specific promoter (ii) an inhibitor of CRISPR, in particular an anti-CRISPR protein such as disclosed above or a nucleic acid encoding thereof and/or iii) a system triggering the degradation of the CRISPR, such as an auxin-degron system.

Preferably, the CRISPR nuclease is not fused with a Spo11 protein.

Preferably, the CRISPR nuclease provided under i) is not fused with:
a Spo11 protein or a variant or fragment thereof such as defined herein; and/or
a Spo11 partner as defined herein, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; and/or
a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; and/or
a DNA binding domain; and/or
a protein/amino acid sequence comprising at least 50 amino acids.

Optionally, the host cell may further comprise a fusion protein comprising a Spo11 protein operably linked to a DNA binding domain, or a nucleic acid encoding said fusion protein.

The eukaryotic host cell may be transformed to introduce heterologous nucleic acid(s), expression cassette(s) or expression vector(s) are described above, by any suitable method known by the skilled person, in particular any method described above.

Alternatively, the method may comprise introducing into the eukaryotic cell the CRISPR nuclease and/or one or more gRNAs and optionally the fusion protein. The CRISPR nuclease and/or the gRNAs and optionally the fusion protein can be introduced into the cytoplasm or nucleus of the eukaryotic cell by any method known to the skilled person, for example by microinjection. In particular, the CRISPR nuclease and optionally the fusion protein can be introduced into the cell as part of a protein-RNA complex comprising one or more gRNA.

In some embodiments, the cell envisioned herein does not comprise a fusion protein comprising i) a CRISPR nuclease as defined above and ii):
a Spo11 protein or a variant or fragment thereof such as defined herein; and/or
a Spo11 partner such as defined herein, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; and/or
a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; and/or
a DNA binding domain, in particular such as described herein; and/or
a protein/amino acid sequence comprising at least 50 amino acids.

In particular, in some embodiments, the cell envisioned herein does not comprise and/or the methods of the invention does not use
a fusion protein comprising a CRISPR nuclease and a Spo11 protein or a variant or fragment thereof as defined herein, or—a fusion protein comprising CRISPR nuclease and a Spo11 partner as defined herein, in particular a protein selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably said variants having at least 80, 85, 90, 95, 96, 97, 98, or at least 99% sequence identity thereto; or
a fusion protein comprising CRISPR nuclease and a topoisomerase, preferably selected from the TOPOVIB or MTOPOVIB family or a variant or orthologue thereof, preferably said variants having at least 80, 85, 90, 95, 96, 97, 98, or at least 99% sequence identity thereto; or
a fusion protein comprising CRISPR nuclease and a DNA binding domain; or
a fusion protein comprising CRISPR nuclease and a protein/amino acid sequence comprising at least 50 amino acids.

In some embodiments, the cell envisioned herein comprises i) a CRISPR nuclease or any fragment or variant thereof, wherein said CRISPR nuclease is not comprised in a fusion protein comprising:
a Spo11 protein or a variant or fragment thereof; and/or
a Spo11 partner, preferably selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably having at least 80, 85, 90, 95, 96, 97, 98, or at least 99% sequence identity thereto; and/or
a topoisomerase, preferably selected from the TOPOVIB or MTOPOVIB family or a variant or orthologue thereof; and/or
a DNA binding domain; and/or
a protein comprising more than 50 amino acids.

Preferably, the cell envisioned herein comprises a CRISPR nuclease or any fragment or variant thereof, wherein said CRISPR nuclease is not comprised in a fusion protein comprising a protein of more than 50 amino acids in length.

Alternatively, the cell envisioned herein comprises:
i) a CRISPR nuclease or any fragment or variant thereof, wherein said CRISPR nuclease is not comprised in a fusion protein comprising:
a Spo11 protein or a variant or fragment thereof; and/or
a Spo11 partner, preferably selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, preferably having at least 80, 85, 90, 95, 96, 97, 98, or at least 99% sequence identity thereto; and/or
a topoisomerase, preferably selected from the TOPOVIB or MTOPOVIB family or a variant or orthologue thereof; and/or
a DNA binding domain; and/or
a protein comprising more than 50 amino acids; and
ii) a fusion protein comprising a Spo11 protein operably linked to a DNA binding domain such as described herein, preferably an inactivated or dead CRISPR nuclease.

Preferably, the cell envisioned herein comprises:
a CRISPR nuclease or any fragment or variant thereof, wherein said CRISPR nuclease is not comprised in a fusion protein comprising a protein of more than 50 amino acids in length; and
a fusion protein comprising a Spo11 protein operably linked to a DNA binding domain such as described herein, preferably an inactivated or dead CRISPR nuclease.

The following examples are presented for illustrative and non-limiting purposes and serve to illustrate the invention.

### EXAMPLES

### Material and Methods

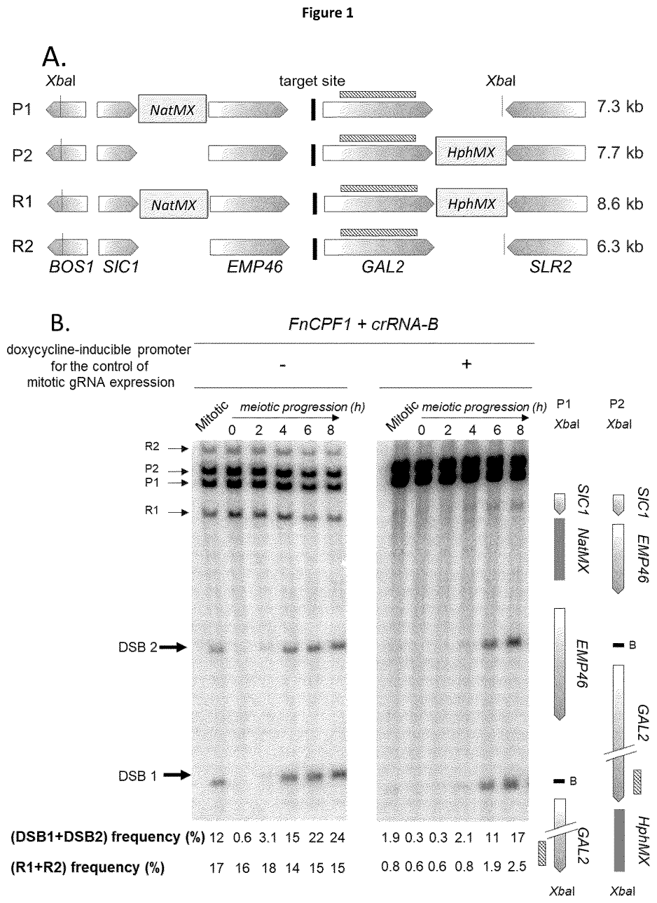

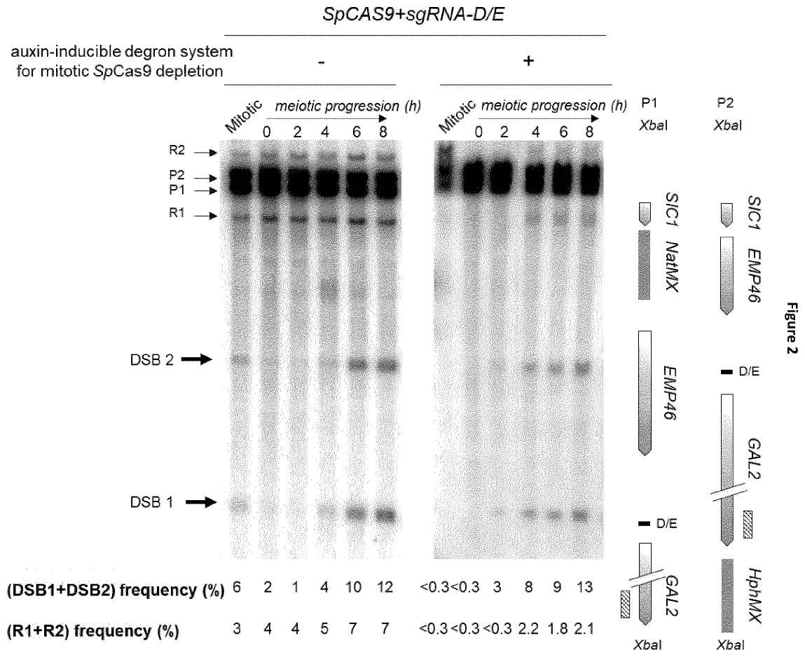

### Southern Blot Analysis of DSBs Targeted in the GAL2 Promoter by FnCpf1 (FIG. 1)

The sequence of the crRNA targeting the B site (5′-GTCCGTGCGGAGATATCTGCGCCGT-3′ SEQ ID NO: 43) in the GAL2 promoter was inserted in a multi-copy plasmid, where expression is governed by the P_{SNR52} promoter (plasmid pAS605). The plasmid pAS632 carries a modified P_{SNR52} promoter where a tetO sequence was inserted, and expresses the TetR gene (TetON system) as described in Bak, G., et al., 2010, BMB Rep., 43, 110-114. The gRNA expression plasmids carrying the LEU2 gene were introduced into yeast cells by electroporation and transformants were selected on plates depleted in leucine (SC-Leu) as described in Sarno et al., 2017, Nucleic Acids Res., 45, e164. The FnCPF1 gene sequence was integrated at the TRP1 locus and its expression is governed by the meiosis-specific REC8 promoter. For meiotic time-courses, the diploid cells were grown at 30° C. in liquid SC-Leu medium, transferred into the SPS pre-sporulation medium, washed with sterile H₂O and transferred into the sporulation medium (1% potassium acetate supplemented with amino acids) as previously described in Sarno et al., 2017, Nucleic Acids Res., 45, e164. In both experiments, doxycycline hyclate was added at a final concentration of 10 μg/ml after transfer of the cells in the sporulation medium (T=0 h). Genomic DNA was prepared from diploid cells harvested during mitotic growth and at 0, 2, 4, 6 and 8 hours after transfer into sporulation medium. After gDNA digestion with XbaI the resulting fragments were separated by electrophoresis in a 0.6% agarose gel and transferred under vacuum onto Hybond XL membrane. The membrane was probed with a labeled ³²P-dCTP PCR products of the GAL2 coding region. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain ANT2906: MATa/α, trp1::P_{REC8}-FnCPF1-TRP1-KanMX/trp1::hisG, spo11::URA3/″, pEMP46::NatMX/0, tGAL2::HphMX/0+plasmid pAS605-crRNA_{pGAL2-B}::LEU2
Strain ANT2942: MATa/α, trp1::P_{REC8}-FnCPF1-TRP1-KanMX/trp1::hisG, spo11::URA3/″, pEMP46::NatMX/0, tGAL2::HphMX/0+plasmid pAS632-crRNA_{pGAL2-B}::LEU2

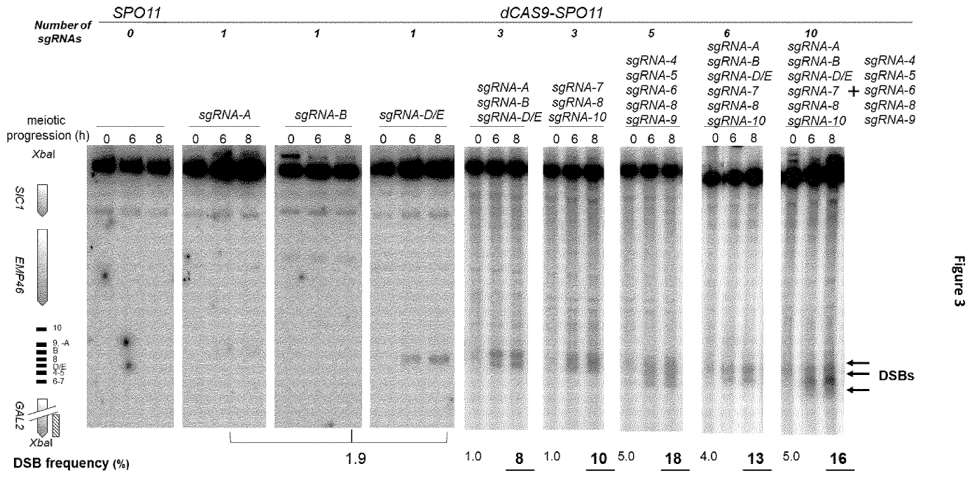

### Southern Blot Analysis of DSBs and Recombinant Molecules Targeted in the GAL2 Region by SpCas9 (FIG. 2)

The sequence of the sgRNA targeting the D/E site (5′-GATCACTCCGAACCGAGATT-3′, SEQ ID NO:46) in the GAL2 promoter was inserted in a multi-copy plasmid, where expression is controlled by a doxycycline-inducible RPR1 promoter (TetON system). This plasmid pAS627 is a derivative of the plasmid pRS416gt such as described in Smith, et al., 2016, Genome Biol., 17, 1-16. The gRNA expression plasmids carrying the LEU2 gene were introduced into yeast cells by electroporation and transformants were selected on plates depleted in leucine (SC-Leu) as described in Sarno et al., 2017, Nucleic Acids Res., 45, e164. The auxin-inducible degron system uses a plant-hormone-induced degradation signal to deplete the protein of interest. Briefly, auxin induces the degradation of proteins that carry the AID* degron sequence by mediating the interaction with the TIR1 protein (see Morawska. & Ulrich 2013, Yeast, 341-351). To target SpCas9 with this auxin-inducible degron system, the SpCas9 protein was C-terminally tagged with the AID* degron, and the rice OsTIR1 gene was integrated at the ARG4 locus (Chromosome VIII). The SpCAS9 coding region, expressed under the meiosis-specific REC8 promoter, was integrated at the TRP1 locus. After transformation with the gRNA expression plasmid, the cells were plated on media depleted in leucine and supplemented with Auxin (1 mM). For meiotic time-courses, the diploid cells were grown at 30° C. in liquid SC-Leu medium, transferred into the SPS pre-sporulation medium, washed with sterile H₂O and transferred into the sporulation medium (1% potassium acetate supplemented with amino acids) as previously described in Sarno et al., 2017, Nucleic Acids Res., 45, e164. Auxin was added at a final concentration of 2 mM in the SC-Leu. Doxycycline hyclate was added at a final concentration of 10 μg/ml after transfer of the cells in the sporulation medium (T=0 h). Genomic DNA was prepared from diploid cells harvested during mitotic growth and at 0, 2, 4, 6 and 8 hours after transfer into sporulation medium. After gDNA digestion with XbaI the resulting fragments were separated by electrophoresis in a 0.6% agarose gel and transferred under vacuum onto Hybond XL membrane. The membrane was probed with a labeled ³²P-dCTP PCR products of the GAL2 coding region. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain ANT3008: MATa/α, trp1::P_{REC8}-SpCAS9-TRP1-KanMX/trp1::hisG, pEMP46::NatMX/0, tGAL2::HphMX/0+plasmid pAS627-sgRNA_{pGAL2-D/E}::LEU2
Strain ANT3146: MATa/α, trp1::P_{REC8}-SpCAS9-AID*-TRP1-KanMX/trp1::hisG, ARG4::OsTIR1/0, pEMP46::NatMX/0, tGAL2::HphMX/0+plasmid pAS627-sgRNA_{pGAL2-D/E}::LEU2

The AID* tag sequence is as described in Morawska & Ulrich, 2013, Yeast, 341-351 and has the sequence 5′-PKDPAKPPAKAQVVGWPPVRSYRKNVMVSCQKSSGGPEAAAFVK*-3′ (SEQ ID No: 24).

Southern Blot Analysis of Targeted DSB Formation in the GAL2 Promoter by dCas9-Spo11 (FIG. 3)

To simultaneously produce poly gRNAs from a primary transcript, the inventors designed a synthetic sequence (purchased from GenScript) which carried a cluster of gRNAs separated by different spacers, in particular such as described in Xie et al., 2015, Proc. Nat. Acad. Sci. USA, 112, 3570-3575 and Zhang et al., 2019, Nat. Commun., 10, 1-10. This polycistronic gene sequence (PGS) consists of tandem repeats of tRNA-gRNA where the tRNA^{Gly} sequence is used to hijack the endogenous tRNA-processing system. As a result, the RNAse P and RNAse Z cleave the primary transcript to excise mature individual gRNAs and tRNA^{Gly}. The released gRNAs direct dCas9-Spo11 to the target sequences. The PGSs were inserted in multi-copy plasmids, and their expression controlled by the doxycycline-inducible RPR1 promoter (TetON system). The 10 spacers included in the 3 PGSs are pGAL2-A: 5′-CAATTCGGAAAGCTTCCTTC-3′, SEQ ID NO: 44, pGAL2-B: 5′-TTGCCTCAGGAAGGCACCGG-3′, SEQ ID NO: 45, pGAL2-D/E: 5′-GATCACTCCGAACCGAGATT-3′, SEQ ID NO: 46, pGAL2-4: 5′-ATCTCAAGATGGGGAGCAAA-3′, SEQ ID NO: 47, pGAL2-5: 5′-TCTTAAATTATACAACGTTC-3′, SEQ ID NO: 48, pGAL2-6: 5′-ACATTTCGCAGGCTAAAATG-3′, SEQ ID NO: 49, pGAL2-7: 5′-CAGTAATTGGATTGAAAATT-3′, SEQ ID NO: 50, pGAL2-8: 5′-GTTCAGGGGTCCATGTGCCT-3′, SEQ ID NO: 51, pGAL2-9: 5′-TTTCTATTAGTAGCTAAAAA-3′, SEQ ID NO: 52 and pGAL2-10: 5′-CAAGGAGGTTTACGGACCAG-3′, SEQ ID NO: 53. The SpdCAS9-SPO11 gene fusion was integrated at the TRP1 locus. Its expression is governed by the constitutive ADH1 promoter as described in Sarno et al., 2017, Nucleic Acids Res., 45, e164. For meiotic time-courses, diploid cells were grown at 30° C. in SC medium depleted in leucine, transferred into SPS pre-sporulation medium, washed with sterile H₂O and transferred into the sporulation medium (1% potassium acetate supplemented with amino acids) as previously described in Sarno et al., 2017, Nucleic Acids Res., 45, e164. The genomic DNA (gDNA) was prepared from diploid cells harvested during mitotic growth and at 0, 2, 4, 6 and 8 hours after transfer into the sporulation medium. The Southern blots were performed as described in FIG. 1. Frequency of DSBs corresponds to the sum of the DSB signal detected in the GAL2 promoter compared to the total signal in the lane.

### Strain Genotypes:

Strain ANT3098: MATa/α, trp1::P_{ADH1}-dCAS9-SPO11-TRP1-KanMX/trp1::hisG, sae2::HphMX/″+plasmid pAS685-sgRNA_{pGAL2-A, -B, -D/E}::LEU2
Strain ANT3099: MATa/α, trp1::P_{ADH1}-dCAS9-SPO11-TRP1-KanMX/trp1::hisG, sae2::HphMX/″+plasmid pAS686-sgRNA_{pGAL2-7, 8, 10}::LEU2
Strain ANT3106: MATa/α, trp1::P_{ADH}-dCCAS9-SPO11-TRP1-KanMX/trp1::hisG, sae2::HphMX/″+plasmid pAS687-sgRNA_{pGAL2-4,5,6,8,9}::LEU2
Strain AND3892: MATa/α, trp1::P_{ADH1}-dCAS9-SPO11-TRP1-KanMX/trp1::hisG, sae2::HphMX/″, ARG4::pAS689-sgRNA_{pGAL2-A, -B, -D/E}/ARG4::pAS690-sgRNA_{7, 8, 10}
Strain ANT3136: MATa/α, trp1::P_{ADH1}-dCAS9-SPO11-TRP1-KanMX/trp1::hisG, sae2::HphMX/″, ARG4::pAS689-sgRNA_{pGAL2-A, -B, -D/E}/ARG4::pAS690-sgRNA_{7,8, 10}+plasmid pAS687-sgRNA_{4,5,6,8,9}::LEU2

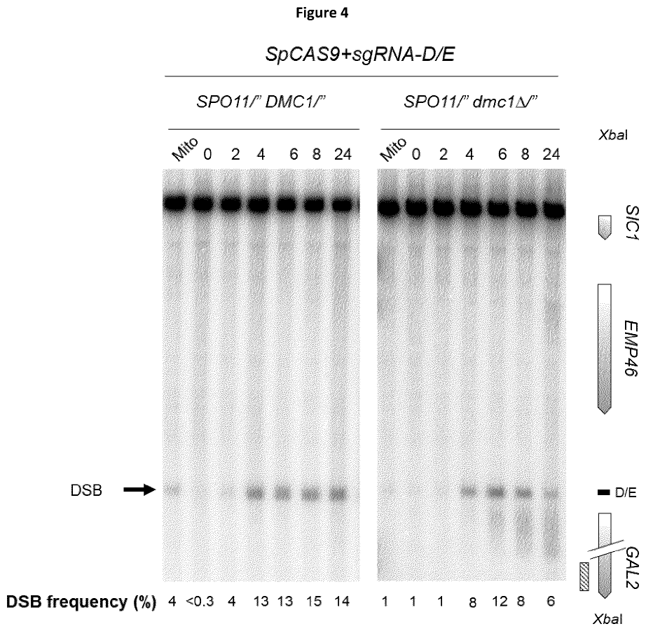

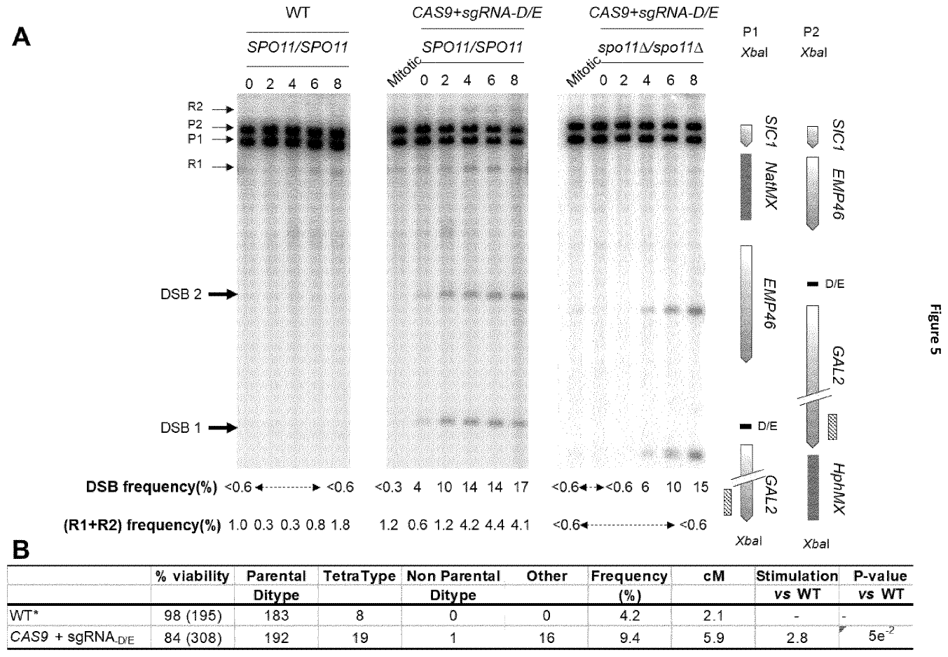

### Southern Blot Analysis of Resected SpCas9-DSBs at the Targeted GAL2 Locus (FIG. 4)

The sequence of the sgRNA targeting the GAL2 D/E site (5′-GATCACTCCGAACCGAGATT-3′, SEQ ID NO:46) in the GAL2 promoter was inserted in a multi-copy plasmid, where expression is controlled by a doxycycline-inducible RPR1 promoter (TetON system). This plasmid pAS627 is a derivative of the plasmid pRS416gt such as described in Smith et al., 2016, Genome Biol., 17, 1-16. The SpCAS9 coding region, expressed under the meiosis-specific REC8 promoter, was integrated at the TRP1 locus. For meiotic time-courses, diploid cells were grown at 30° C. in SC medium depleted in leucine (SC-Leu), transferred into SPS pre-sporulation medium, washed with sterile H₂O and transferred into the sporulation medium (1% potassium acetate supplemented with amino acids) as previously described in Sarno et al., 2017, Nucleic Acids Res., 45, e164. Doxycycline hyclate was added at a final concentration of 10 μg/ml after transfer of the cells in the sporulation medium (T=0 h). Genomic DNA was prepared from diploid cells harvested during mitotic growth and at 0, 2, 4, 6, 8 and 24 hours after transfer into the sporulation medium. The yeast genomic DNA was digested with XbaI. The resulting fragments were separated by electrophoresis in a 0.8% agarose gel and transferred under vacuum onto Hybond XL membrane. The membrane was probed with a labeled ³²P-dCTP PCR products of the GAL2 coding region. DSB frequency corresponds to the sum of the radioactivity in the DSB bands compared to the total signal in the lane.

### Strain Genotypes:

Strain ANT3100: MATa/α, trp1::P_{REC8}-SpCAS9-TRP1-KanMX/trp1::hisG, SPO11/″, DMC1/″+plasmid pAS627-sgRNA_{pGAL2-D/E}::LEU2
Strain ANT3096: MATa/α, trp1::P_{REC8}-SpCAS9-TRP1-KanMX/trp1::hisG, spo11::URA3/SPO11, dmc1Δ/″+plasmid pAS627-sgRNA_{pGAL2-D/E}::LEU2

### Southern Blot Analysis of Targeted SpCas9-DSB Formation in the Targeted GAL2 Region in SPO11 Strain but not in Spo11A Strain (FIG. 5)

The sequence of the sgRNA targeting the GAL2 D/E site (5′-GATCACTCCGAACCGAGATT-3′, SEQ ID NO:46) in the GAL2 promoter was inserted in a multi-copy plasmid, where expression is controlled by a doxycycline-inducible RPR1 promoter (TetON system). This plasmid pAS627 is a derivative of the plasmid pRS416gt such as described in Smith et al., 2016, Genome Biol, 17, 1-16). The SpCAS9 coding region, expressed under the meiosis-specific REC8 promoter, was integrated at the TRP1 locus. For meiotic time-courses, diploid cells were grown at 30° C. in SC medium depleted in leucine (SC-Leu), transferred into SPS pre-sporulation medium, washed with sterile H₂O and transferred into the sporulation medium (1% potassium acetate supplemented with amino acids) as previously described in Sarno et al., 2017, Nucleic Acids Res., 45, e164. Doxycycline hyclate was added at a final concentration of 10 μg/ml after transfer of the cells in the sporulation medium (T=0 h). Genomic DNA was prepared from diploid cells harvested during mitotic growth and at 0, 2, 4, 6 and 8 hours after transfer into the sporulation medium. The yeast genomic DNA was digested with XbaI. The resulting fragments were separated by electrophoresis in a 0.6% agarose gel and transferred under vacuum onto Hybond XL membrane. The membrane was probed with a labeled ³²P-dCTP PCR products of the GAL2 coding region. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain AND2895: MATa/α, trp1::hisG/″, SPO11/″, pEMP46::NatMX/0, tGAL2::HphMX/0
Strain ANT3008: MATa/α, trp1::P_{REC8}-SpCAS9-TRP1-KanMX/trp1::hisG, SPO11/″, pEMP46::NatMX/0, tGAL2::HphMX/0+plasmid pAS627-sgRNA_{pGAL2-D/E}::LEU2
Strain ANT3929: MATa/α, trp1::P_{REC8}-SpCAS9-TRP1-KanMX/trp1::hisG, spo11::URA3/″, pEMP46::NatMX/0, tGAL2::HphMX/0+plasmid pAS627-sgRNA_{pGAL2-D/E}::LEU2

The NatMX and HphMX genes confer nourseothricin (Nat^{R}) and hygromycine (Hygro^{R}) resistance, respectively. In the absence of recombination between the NatMX and HphMX markers the 4 meiosis products are parental ditype (PD: 2 Nat^{R} Hygros and 2 Nat^{s} Hygro^{R}). A crossover event between the markers yield tetratype tetrads (TT: 1 Nat^{R} Hygros, 1 Nat^{s} Hygro^{R}, 1 Nat^{s} Hygro^{s} and 1 Nat^{R} Hygro^{R}) and 2 crossover events involving the 4 chromatids in the same meiotic cell yield non-parental ditype tetrads (NPD: 2 Nat^{R} Hygro^{R} and 2 Nats Hygros). Other types of segregation (others) were observed. These complex events result in non-Mendelian segregation of the markers (4:0 and/or 0:4) suggesting the existence of recombination events that occurred before meiosis. Additional 3:1 and 1:3 marker segregation revealed the gene conversion of the NatMX and/or HphMX markers. 191 and 228 WT and CAS9 4-spore tetrads were dissected, respectively. The genetic distance was determined according to the formula cM=100(T+6NPD)/2(PD/T+NPD). The difference in the recombination frequency between the wild-type and SpCAS9 strains is statistically different (P-value<0.06; two-tailed Fischer's exact test).

In strain ANT3008, the sequence of the sgRNA targeting the GAL2 D/E site (5′-GATCACTCCGAACCGAGATT-3′, SEQ ID NO: 46) in the GAL2 promoter was inserted in a multi-copy plasmid, where expression is controlled by a doxycycline-inducible RPR1 promoter (TetON system). This plasmid pAS627 is a derivative of the plasmid pRS416gt such as described in Smith et al., 2016, Genome Biol., 17, 1-16). The SpCAS9 coding region, expressed under the meiosis-specific REC8 promoter, was integrated at the TRP1 locus. For meiotic time-courses, diploid cells were grown at 30° C. in SC medium depleted in leucine (SC-Leu), transferred into SPS pre-sporulation medium, washed with sterile H₂O and transferred into the sporulation medium (1% potassium acetate supplemented with amino acids) as previously described in Sarno et al., 2017, Nucleic Acids Res., 45, e164. Doxycycline hyclate was added at a final concentration of 10 μg/ml after transfer of the cells in the sporulation medium (T=0 h). After sporulation of strains AND2895 (wild type control) and ANT3008, four-spore tetrads (191 and 228 for WT and CAS9, respectively) were dissected and genotyped for the segregation of the antibiotic resistance cassettes (hygromycin and nourseothricin for the HphMX and NatMX cassettes, respectively). The number of parental ditype (PD) tetrads was compared to that of tetratypes (T) and non-parental ditypes (NPD), in which the drug resistant markers segregated as 2:2 (mendelian segregation) per tetrad, The genetic distance was determined according to the formula cM=100(T+6NPD)/2(PD/T+NPD). Recombination rates were compared using a two-failed Fischer's exact test. Other tetrads include cases in which the pattern of segregation of the drug resistant markers were more complex with a combination of 4:0, 0:4, 3:1, 1:3 and/or 2:2 segregation resulting from mitotic recombination events or multi-chromatids (2, 3 and 4) meiotic events.

### Strain Genotypes:

Strain AND2895: MATa/α, trp1::hisG/″, SPO11/″, pEMP46::NatMX/0, tGAL2::HphMX/0
Strain ANT3008: MATa/α, trp1::P_{REC8}-SpCAS9-TRP1-KanMX/trp1::hisG, SPO11/″, pEMP46::NatMX/0, tGAL2::HphMX/0+plasmid pAS627-sgRNA_{pGAL2-D/E}::LEU2

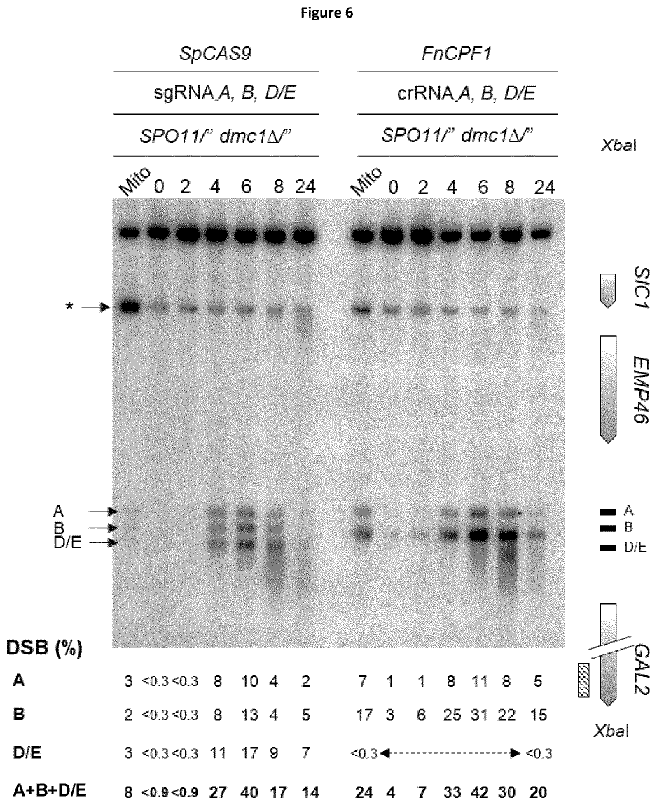

### Southern Blot Analysis of Poly-Targeting of SpCas9 and FnCpf1 in the GAL2 Promoter (FIG. 6)

To express the sgRNAs targeting the GAL2-A, -B and -D/E sites the corresponding sgRNA expression cassettes were inserted into the pAS522 plasmid. Each sgRNA expression cassette contains the RPR1 promoter and terminator, and a gRNA sequence (GAL2-A: 5′-CAATTCGGAAAGCTTCCTTC-3′, SEQ ID NO: 44; GAL2-B: 5′-TTGCCTCAGGAAGGCACCGG-3′, SEQ ID NO: 45 and GAL2-D/E: 5′-GATCACTCCGAACCGAGATT-3′, SEQ ID NO: 46). The multi-copy plasmid pAS522 is described in Sarno et al., 2017, Nucleic Acids Res., 45, e164. To target FnCpf1 to the GAL2-A, —B and -D/E sites an artificial CRISPR pre-crRNA array consisting of 3 spacers (GAL2-A: 5′-AGAAAAGGTATTCAACGTCAATTCG-3′, SEQ ID NO: 54; GAL2-B: 5′-GTCCGTGCGGAGATATCTGCGCCGT-3′, SEQ ID NO: 43 and GAL2-D/E: 5′-CTCCCCATCTTGAGATGGGAAGGGC-3′, SEQ ID NO: 55) separated by direct repeats from the locus of Francisella novicida was constructed as described in Zetsche et al., 2015, Cell, 163, 759-771 and cloned into the multi-copy pAS633 plasmid. crRNA expression is governed by the SNR52 promoter. The SpCAS9 and FnCPF1 coding regions, expressed under the meiosis-specific REC8 promoter, were integrated at the TRP1 locus. For meiotic time-courses, diploid cells were grown at 30° C. in SC medium depleted in leucine (SC-Leu), transferred into SPS pre-sporulation medium, washed with sterile H₂O and transferred into the sporulation medium (1% potassium acetate supplemented with amino acids) as previously described in Sarno et al., 2017, Nucleic Acids Res., 45, e164. Genomic DNA was prepared from diploid cells harvested during mitotic growth (Mito) and at 0, 2, 4, 6, 8 and 24 hours after transfer into the sporulation medium. The yeast genomic DNA was digested with XbaI. The resulting fragments were separated by electrophoresis in a 0.8% agarose gel and transferred under vacuum onto Hybond XL membrane. The membrane was probed with a labeled ³²P-dCTP PCR products of the GAL2 coding region. DSB frequency corresponds to the sum of the radioactivity in the DSB bands compared to the total signal in the lane.

### Strain Genotypes:

Strain ANT3164: MATa/α, trp1::P_{REC8}-SpCAS9-TRP1-KanMX/trp1::hisG, SPO11/″, dmc1Δ/″+plasmid pAS522-sgRNA_{pGAL2-A, -B, -D/E}::LEU2
Strain ANT3165: MATa/la, trp1::P_{REC8}-FnCPF1-TRP1-KanMX/trp1::hisG, SPO11/″, dmc1Δ/″+plasmid pAS633-crRNA_{pGAL2-A, -B, -D/E}::LEU2

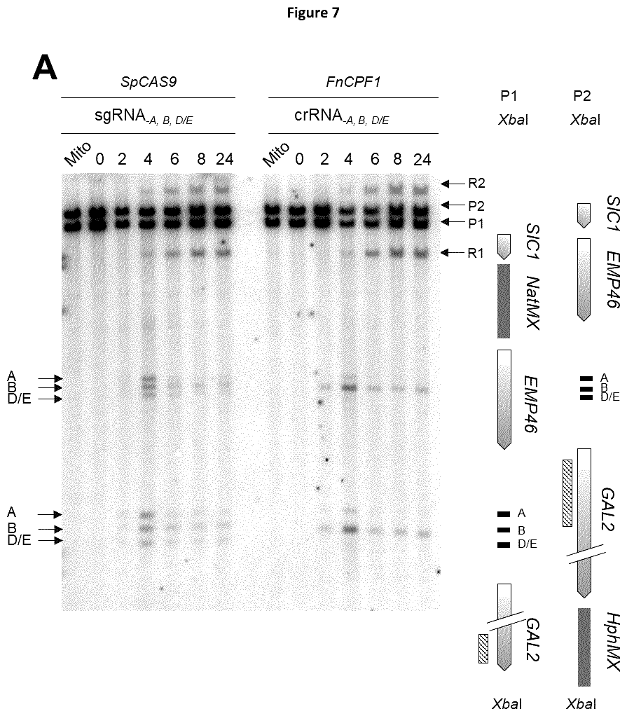

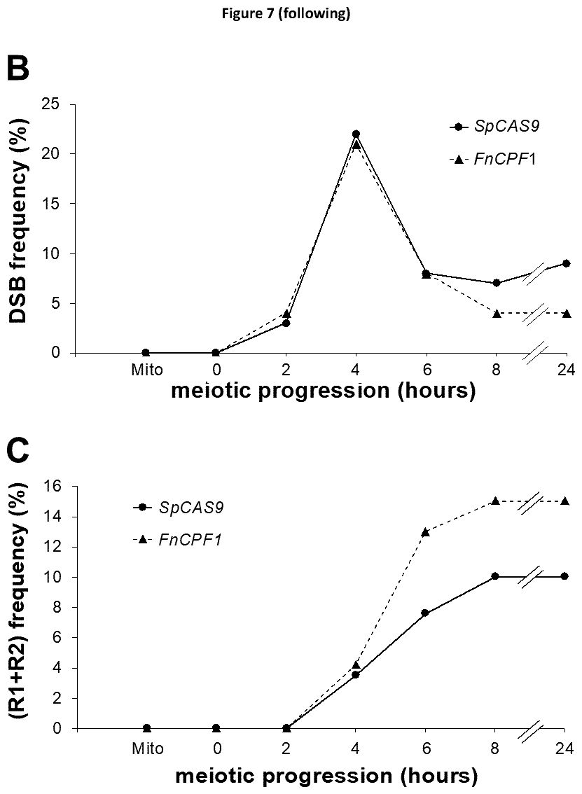

### Southern Blot Analysis of DSB and Recombinant Molecules in the GAL2 Region by Poly-Targeting of SpCas9 and FnCpf1 (FIG. 7)

To express the sgRNAs targeting the GAL2-A, -B and -D/E sites the corresponding sgRNA expression cassettes were inserted into the pAS522 plasmid. Each sgRNA expression cassette contains the RPR1 promoter and terminator, and a gRNA sequence (GAL2-A: 5′-CAATTCGGAAAGCTTCCTTC-3′, SEQ ID NO: 44; GAL2-B: 5′-TTGCCTCAGGAAGGCACCGG-3′, SEQ ID NO: 45 and GAL2-D/E: 5′-GATCACTCCGAACCGAGATT-3′, SEQ ID NO: 46). The multi-copy plasmid pAS522 is described in Sarno et al., 2017, Nucleic Acids Res., 45, e164. To target FnCpf1 to the GAL2-A, —B and -D/E sites an artificial CRISPR pre-crRNA array consisting of 3 spacers (GAL2-A: 5′-AGAAAAGGTATTCAACGTCAATTCG-3′, SEQ ID NO: 54; GAL2-B: 5′-GTCCGTGCGGAGATATCTGCGCCGT-3′, SEQ ID NO: 43 and GAL2-D/E: 5′-CTCCCCATCTTGAGATGGGAAGGGC-3′, SEQ ID NO: 55) separated by direct repeats from the locus of Francisella novicida was constructed as described in Zetsche et al., 2015, Cell, 163, 759-771 and cloned into the multi-copy pAS633 plasmid. crRNA expression is governed by the SNR52 promoter. The SpCAS9 and FnCPF1 coding regions, expressed under the meiosis-specific REC8 promoter, was integrated at the TRP1 locus. The auxin-inducible degron system uses a plant-hormone-induced degradation signal to deplete the protein of interest. Briefly, auxin induces the degradation of proteins that carry the AID* degron sequence by mediating the interaction with the TIR1 protein (Morawska & Ulrich, 2013, Yeast, 30: 341-351). To target SpCas9 and FnCpf1 with this auxin-inducible degron system, both proteins were C-terminally tagged with the AID* degron, and the rice OsTIR1 gene was integrated at the ARG4 locus (Chromosome VIII). After transformation with the gRNA expression plasmid, the cells were plated on media depleted in leucine and supplemented with Auxin (1 mM). For meiotic time-courses, the diploid cells were grown at 30° C. in liquid SC-Leu medium supplemented with 2 mM Auxin, transferred into the SPS pre-sporulation medium, washed with sterile H₂O and transferred into the sporulation medium (1% potassium acetate supplemented with amino acids) as previously described in Sarno et al., 2017, Nucleic Acids Res., 45, e164. Auxin was added at a final concentration of 2 mM 5 hours after transfer of the cells in the sporulation medium (T=5 hours). Genomic DNA was prepared from diploid cells harvested during mitotic growth (Mito) and at 0, 2, 4, 6, 8 and 24 hours after transfer into the sporulation medium. The yeast genomic DNA was digested with XbaI. The resulting fragments were separated by electrophoresis in a 0.6% agarose gel and transferred under vacuum onto Hybond XL membrane. The membrane was probed with a labeled ³²P-dCTP PCR products of the GAL2 coding region. DSB frequency corresponds to the sum of the radioactivity in the DSB1+DSB2 bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain ANT3162: MATa/α, trp1::P_{REC8}-SpCAS9-AID*-TRP1-KanMX/trp1::hisG, ARG4::OsTIR1/0, pEMP46::NatMX/0, tGAL2::HphMX/0+plasmid pAS522-sgRNA_{pGAL2-A,-B,D/E}::LEU2
Strain ANT3163: MATa/α, trp1::P_{REC8}-FnCPF1-AID*-TRP1-KanMX/trp1::hisG, ARG4::OsTIR1/0, pEMP46::NatMX/0, tGAL2::HphMX/0+plasmid pAS633-crRNA_{pGAL2-A,-B,D/E}::LEU2

The AID* tag sequence is as described in Morawska & Ulrich, 2013, Yeast, 30, 341-351 and has the sequence 5′-PKDPAKPPAKAQVVGWPPVRSYRKNVMVSCQKSSGGPEAAAFVK*-3′ (SEQ ID No: 24).

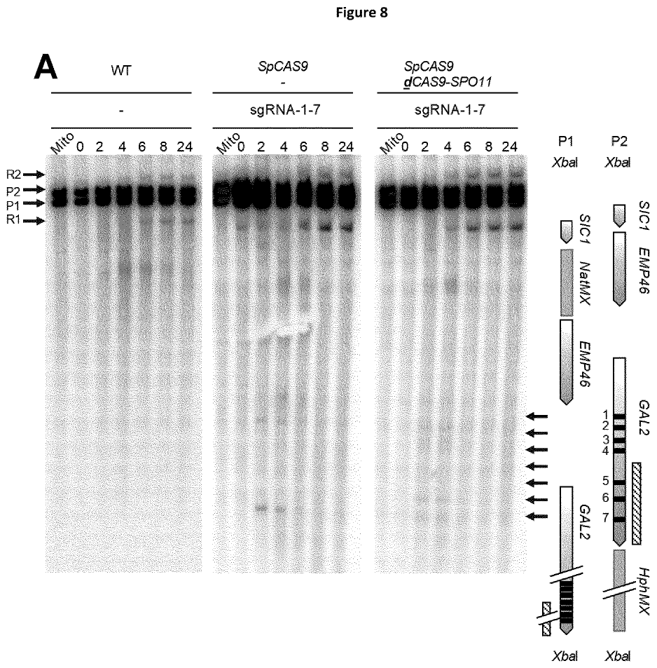

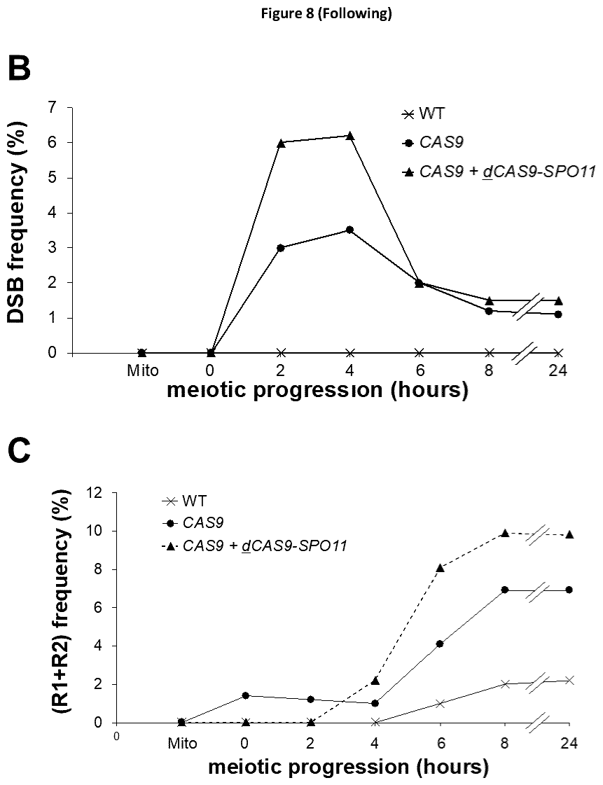

### Southern Blot Analysis of DSB and Recombinant Molecules in the GAL2 Region by Poly-Targeting of SpCas9 and SpdCas9-Spo11 (FIG. 8)

To simultaneously produce poly gRNAs from a primary transcript, the inventors designed a synthetic sequence (purchased from GenScript) which carried a cluster of gRNAs separated by different spacers, in particular such as described in Xie, et al., 2015, Proc. Nat. Acad. Sci. USA, 112, 3570-3575) and Zhang et al., 2019, Nat. Commun. 10, 1053). This polycistronic gene sequence (PGS) consists of tandem repeats of tRNA-gRNA where the tRNA^{Gly} sequence is used to hijack the endogenous tRNA-processing system. As a result, the RNAse P and RNAse Z cleave the primary transcript to excise mature individual gRNAs and tRNA^{Gly}. The released gRNAs direct Cas9 and dCas9-Spo11 to the target sequences. The 7 sgRNAs targeting the GAL2 promoter were expressed from the multi-copy pAS707 plasmid, that carries the synthetic PGS and the sgRNA-4 expression cassette. sgRNA expression is controlled by the doxycycline-inducible RPR1 promoter (TetON system). The 6 spacers included in the PGS are GAL2-ORF-1: 5′-AAGTGGTACCAATATTTCAT-3′, SEQ ID NO: 56, GAL2-ORF-2: 5′-TTGGGCTACTGTACTAATTA-3′, SEQ ID NO: 57, GAL2-ORF-3: 5′-ATCCCCACGTTATTTATGTG-3′, SEQ ID NO: 58, GAL2-ORF-5: 5′-GCGTCGTAAATGTTTACTTT-3′, SEQ ID NO: 60, GAL2-ORF-6: 5′-TTTTCTGTTATGCCACAACC-3′, SEQ ID NO: 61 and GAL2-ORF-7: 5′-ATAACCGTAGTAGAAGTTAA-3′, SEQ ID NO: 62. The sgRNA-4 expressed from the sgRNA expression cassette (containing the RPR1 promoter and terminator, and a gRNA sequence) displays the spacer GAL2-ORF-4: 5′-TAGTGCACTTACCCCACGTT-3′, SEQ ID NO: 58. The SpCAS9 and SpdCAS9-SPO11 gene fusions were integrated at the TRP1 locus on homologous chromosomes. Their expression is governed by the meiosis-specific REC8 and constitutive ADH1 promoters, respectively. The auxin-inducible degron system uses a plant-hormone-induced degradation signal to deplete the protein of interest. Briefly, auxin induces the degradation of proteins that carry the AID* degron sequence by mediating the interaction with the TIR1 protein (Morawska & Ulrich, 2013, Yeast, 30, 341-351). To target SpCas9 with this auxin-inducible degron system, the protein was C-terminally tagged with the AID* degron, and the rice OsTIR1 gene was integrated at the ARG4 locus (Chromosome VIII). After transformation with the gRNA expression plasmid, the cells were plated on media depleted in leucine and supplemented with Auxin (1 mM). For meiotic time-courses, the diploid cells were grown at 30° C. in liquid SC-Leu medium supplemented with 2 mM Auxin, transferred into the SPS pre-sporulation medium, washed with sterile H₂O and transferred into the sporulation medium (1% potassium acetate supplemented with amino acids) as previously described in Sarno et al., 2017, Nucleic Acids Res., 45, e164. Auxin was added at a final concentration of 2 mM 5h hours after transfer of the cells in the sporulation medium (T=5 hours). Genomic DNA was prepared from diploid cells harvested during mitotic growth (Mito) and at 0, 2, 4, 6, 8 and 24 hours after transfer into the sporulation medium. The yeast genomic DNA was digested with XbaI. The resulting fragments were separated by electrophoresis in a 0.6% agarose gel and transferred under vacuum onto Hybond XL membrane. The membrane was probed with a labeled ³²P-dCTP PCR products of the GAL2 coding region. DSB frequency corresponds to the sum of the radioactivity in the DSB bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain AND3806: MATa/la, trp1::hisG/″, pEMP46::NatMX/0, tGAL2::HphMX/0
Strain ANT3183: MATa/α, trp1::P_{REC8}-SpCAS9-AID*-TRP1-KanMX/trp1::hisG, ARG4::OsTIR1/0, pEMP46::NatMX/0, tGAL2::HphMX/0+plasmid pAS707-sgRNA_{GAL2-1-7}::LEU2
Strain ANT3184: MATa/α, trp1::P_{REC8}-SPCAS9-AID*-TRP1-KanMX/trp1::P_{ADH1}-SpdCAS9-SPO11-TRP1-KanMX, ARG4::OsTIR/0, pEMP46::NatMX/0, tGAL2::HphMX/0+plasmid pAS707-sgRNA_{GAL2-1-7}::LEU2

The AID* tag sequence is as described in Morawska & Ulrich, 2013, Yeast, 30, 341-351 and has the sequence 5′-PKDPAKPPAKAQVVGWPPVRSYRKNVMVSCQKSSGGPEAAAFVK*-3′ (SEQ ID No: 24).

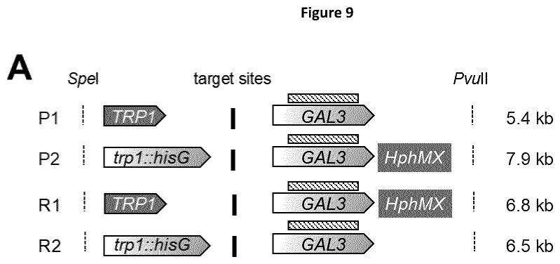

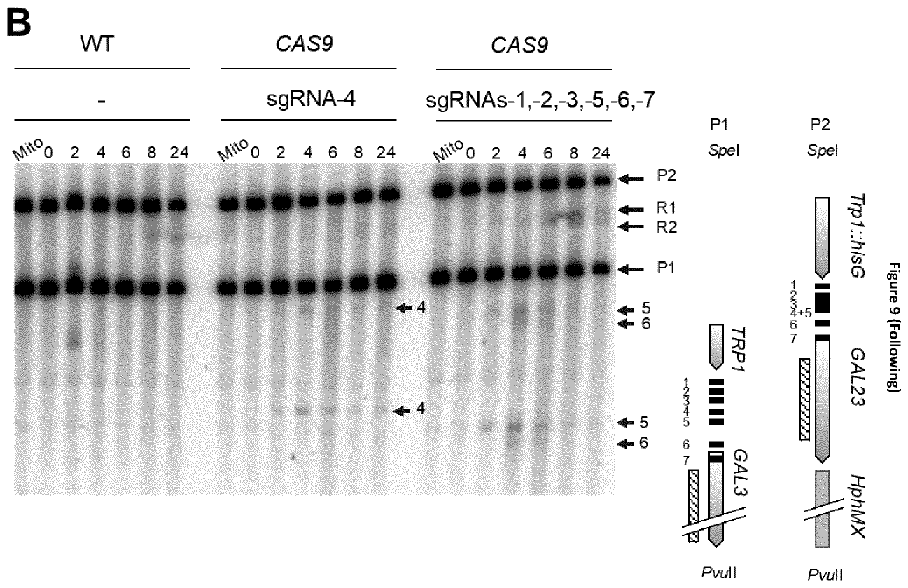

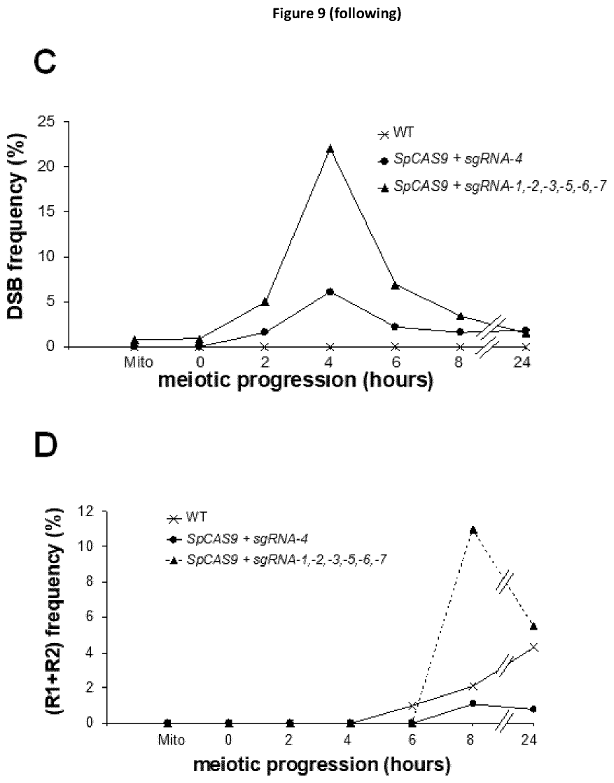

### Southern Blot Analysis of DSB and Recombinant Molecules in the GAL3 Region by Poly-Targeting of SpCas9 (FIG. 9)

To simultaneously produce poly gRNAs from a primary transcript, the inventors designed a synthetic sequence (purchased from GenScript) which carried a cluster of gRNAs separated by different spacers, in particular such as described in Xie et al., 2015, Proc. Nat. Acad. Sci. USA, 112, 3570-3575 and Zhang et al., 2019, Nat. Commun. 10, 1053. This polycistronic gene sequence (PGS) consists of tandem repeats of tRNA-gRNA where the tRNA^{Gly} sequence is used to hijack the endogenous tRNA-processing system. As a result, the RNAse P and RNAse Z cleave the primary transcript to excise mature individual gRNAs and tRNA^{Gly}. The released gRNAs direct Cas9 to the target sequences. sgRNA-pGAL3-4 (SEQ ID NO: 65) and the 6 additional sgRNAs targeting the GAL3 promoter were expressed from the multi-copy pAS699 and pAS705 plasmids, respectively. The spacer sequence of sgRNA-pGAL3-4 is 5′-TAGTGCACTTACCCCACGTT-3′, SEQ ID NO: 66. The 6 spacers included in the PGS are pGAL3-1: 5′-AAAGACAATGCCAAATCATT-3′, SEQ ID NO: 63, pGAL3-2: 5′-GATTCTTGCTAGCCTTTTCT-3′, SEQ ID NO: 64, pGAL3-3: 5′-GTAGAAGATAATAGTAAAAG-3′, SEQ ID NO: 65, pGAL3-5: 5′-ATTGACCGCCTGAAACACAT-3′, SEQ ID NO: 67, pGAL3-6: 5′-GCACTCCTGATTCCGCTAAT-3′, SEQ ID NO: 68 and pGAL3-7: 5′-CGATAAAATCAGGTTTGACA-3′, SEQ ID NO: 69. sgRNA expression is controlled by the doxycycline-inducible RPR1 promoter (TetON system). The SpCAS9 gene was integrated at the URA3 locus. Its expression is governed by the meiosis-specific REC8 promoter. The auxin-inducible degron system uses a plant-hormone-induced degradation signal to deplete the protein of interest. Briefly, auxin induces the degradation of proteins that carry the AID* degron sequence by mediating the interaction with the TIR1 protein (Morawska & Ulrich, 2013, Yeast 30, 341-351). To target SpCas9 with this auxin-inducible degron system, the protein was C-terminally tagged with the AID* degron, and the rice OsTIR1 gene was integrated at the ARG4 locus (Chromosome VIII). After transformation with the gRNA expression plasmid, the cells were plated on media depleted in leucine and supplemented with Auxin (1 mM). For meiotic time-courses, the diploid cells were grown at 30° C. in liquid SC-Leu medium supplemented with 2 mM Auxin, transferred into the SPS pre-sporulation medium, washed with sterile H₂O and transferred into the sporulation medium (1% potassium acetate supplemented with amino acids) as previously described in Sarno et al., 2017, Nucleic Acids Res., 45, e164. Auxin was added at a final concentration of 2 mM 5h hours after transfer of the cells in the sporulation medium (T=5 hours). Genomic DNA was prepared from diploid cells harvested during mitotic growth (Mito) and at 0, 2, 4, 6, 8 and 24 hours after transfer into the sporulation medium. The yeast genomic DNA was digested with SpeI and PvuII. The resulting fragments were separated by electrophoresis in a 0.6% agarose gel and transferred under vacuum onto Hybond XL membrane. The membrane was probed with a labeled ³²P-dCTP PCR products of the GAL3 coding region. DSB frequency corresponds to the sum of the radioactivity in the DSB bands compared to the total signal in the lane. Frequency of recombinant molecules was measured as the percentage of radioactivity in R1+R2 relative to the total amount of radioactivity in the parental P1+P2 plus recombinant R1+R2 bands.

### Strain Genotypes:

Strain AND3936: MATa/α, TRP1/trp1::hisG, tGAL3::HphMX/0
Strain ANT3187: MATa/α, ura3::P_{REC8}-SpCAS9-AID*-TRP1-KanMX/URA3, TRP1/trp1::hisG, ARG4::OsTIR1/0, tGAL3::HphMX/0, +plasmid pAS699-sgRNA_{pGAL3-4}::LEU2
Strain ANT3190: MATa/α, ura3::P_{REC8}-SpCAS9-AID*-TRP1-KanMX/URA3, TRP1/trp1::hisG, ARG4::OsTIR1/0, tGAL3::HphMX/0, +plasmid pAS705-sgRNAp_{GAL3-1,-2,-3,-5,-6,-7}::LEU2

The AID* tag sequence is as described in Morawska & Ulrich, 2013, Yeast, 30, 341-351 and has the sequence 5′-PKDPAKPPAKAQVVGWPPVRSYRKNVMVSCQKSSGGPEAAAFVK*-3′ (SEQ ID No: 24).

TABLE 1
Summary of CRISPR-targeted genomic sites in the GAL2 promoter.
The first three columns indicate the plasmids used in the
experiments, the name of the gRNAs expressed from the
plasmids and the SEQ ID number of the gRNA sequence, re-
spectively. The last four columns indicate the 5′ to 3′
sequence targeted by the corresponding sgRNA, the
coordinates on chromosome XII, the strand and the SEQ ID
number of the target sequence, respectively, “Chr” stands
for “Chromosome”.

| | | SEQ | | SEQ | | Coordi- | |
|---|---|---|---|---|---|---|---|
| Plasmid | gRNA | ID | Target sequence | ID | Chr | nates | Strand |
| pAS633 | pGAL2 | 54 | CGAATTGACGTTGAATACCTTTTCT | 70 | XII | 289683- | − |
| | crRNA- | | | | | 289659 | |
| | A | | | | | | |
| pAS605, | pGAL2 | 43 | ACGGCGCAGATATCTCCGCACGGAC | 71 | XII | 289832- | − |
| pAS632 | crRNA- | | | | | 289808 | |
| and | B | | | | | | |
| 633 | | | | | | | |
| pAS633 | pGAL2 | 55 | GCCCTTCCCATCTCAAGATGGGGAG | 72 | XII | 289912- | + |
| | crRNA- | | | | | 289936 | |
| | D/E | | | | | | |
| pAS522, | pGAL2 | 44 | GAAGGAAGCTTTCCGAATTG | 73 | XII | 289696- | − |
| pAS685 | gRNA- | | | | | 289677 | |
| and | A | | | | | | |
| pAS689 | | | | | | | |
| pAS522, | pGAL2 | 45 | CCGGTGCCTTCCTGAGGCAA | 74 | XII | 289798- | − |
| pAS685 | gRNA- | | | | | 289779 | |
| and | B | | | | | | |
| pAS689 | | | | | | | |
| pAS522, | pGAL2 | 46 | AATCTCGGTTCGGAGTGATC | 75 | XII | 289905- | − |
| pAS627, | gRNA- | | | | | 289886 | |
| pAS685 | D/E | | | | | | |
| and | | | | | | | |
| pAS689 | | | | | | | |
| pAS687 | pGAL2 | 47 | TTTGCTCCCCATCTTGAGAT | 76 | XII | 289940- | − |
| | gRNA- | | | | | 289921 | |
| | 4 | | | | | | |
| pAS687 | pGAL2 | 48 | GAACGTTGTATAATTTAAGA | 77 | XII | 290000- | − |
| | gRNA- | | | | | 289981 | |
| | 5 | | | | | | |
| pAS687 | pGAL2 | 49 | CATTTTAGCCTGCGAAATGT | 78 | XII | 290045- | − |
| | gRNA- | | | | | 290026 | |
| | 6 | | | | | | |
| pAS686 | pGAL2 | 50 | AATTTTCAATCCAATTACTG | 79 | XII | 290103- | − |
| and | gRNA- | | | | | 290084 | |
| pAS690 | 7 | | | | | | |
| pAS686, | pGAL2 | 51 | AGGCACATGGACCCCTGAAC | 80 | XII | 289850- | − |
| pAS687 | gRNA- | | | | | 289831 | |
| and | 8 | | | | | | |
| pAS690 | | | | | | | |
| pAS687 | pGAL2 | 52 | TTTTTAGCTACTAATAGAAA | 81 | XII | 289746- | − |
| | gRNA- | | | | | 289727 | |
| | 9 | | | | | | |
| pAS686 | pGAL2 | 53 | CTGGTCCGTAAACCTCCTTG | 82 | XII | 289537- | − |
| and | gRNA- | | | | | 289518 | |
| pAS690 | 10 | | | | | | |
| pAS707 | GAL2 | 56 | ATGAAATATTGGTACCACTT | 83 | XII | 290744- | − |
| | sgRNA- | | | | | 290725 | |
| | 1 | | | | | | |
| pAS707 | GAL2 | 57 | TAATTAGTACAGTAGCCCAA | 84 | XII | 290900- | − |
| | sgRNA- | | | | | 290881 | |
| | 2 | | | | | | |
| pAS707 | GAL2 | 58 | CACATAAATAACGTGGGGAT | 85 | XII | 291022- | − |
| | sgRNA- | | | | | 291003 | |
| | 3 | | | | | | |
| pAS707 | GAL2 | 59 | TAGTGCACTTACCCCACGTT | 86 | XII | 291122- | − |
| | sgRNA- | | | | | 291103 | |
| | 4 | | | | | | |
| pAS707 | GAL2 | 60 | AAAGTAAACATTTACGACGC | 87 | XII | 291403- | − |
| | sgRNA- | | | | | 291387 | |
| | 5 | | | | | | |
| pAS707 | GAL2 | 61 | GGTTGTGGCATAACAGAAAA | 88 | XII | 291558- | − |
| | sgRNA- | | | | | 291539 | |
| | 6 | | | | | | |
| pAS707 | GAL2 | 62 | TTAACTTCTACTACGGTTAT | 89 | XII | 291698- | + |
| | sgRNA- | | | | | 291717 | |
| | 7 | | | | | | |
| pAS705 | pGAL3 | 63 | AATGATTTGGCATTGTCTTT | 90 | IV | 462646- | − |
| | sgRNA- | | | | | 462627 | |
| | 1 | | | | | | |
| pAS705 | pGAL3 | 64 | AGAAAAGGCTAGCAAGAATC | 91 | IV | 462788- | − |
| | sgRNA- | | | | | 462769 | |
| | 2 | | | | | | |
| pAS705 | pGAL3 | 65 | CTTTTACTATTATCTTCTAC | 92 | IV | 462935- | + |
| | sgRNA- | | | | | 462954 | |
| | 3 | | | | | | |
| pAS699 | pGAL3 | 66 | AACGTGGGGTAAGTGCACTA | 93 | IV | 463145- | − |
| | sgRNA- | | | | | 463126 | |
| | 4 | | | | | | |
| pAS705 | pGAL3 | 67 | ATGTGTTTCAGGCGGTCAAT | 94 | IV | 463270- | − |
| | sgRNA- | | | | | 463251 | |
| | 5 | | | | | | |
| pAS705 | pGAL3 | 68 | ATTAGCGGAATCAGGAGTGC | 95 | IV | 463372- | + |
| | sgRNA- | | | | | 463391 | |
| | 6 | | | | | | |
| pAS705 | pGAL3 | 69 | TGTCAAACCTGATTTTATCG | 96 | IV | 463538- | + |
| | sgRNA- | | | | | 463557 | |
| | 7 | | | | | | |

### Results

TetON-Controlled Expression of gRNAs Reduces the Mitotic Leak of FnCpf1 Activity. (FIG. 1)

In order to reduce the mitotic leak of FnCpf1 activity that leads to the formation of targeted DSBs (12%) and recombinant molecules (17%) during the mitotic growth, the inventors expressed the crRNA under the control of a doxycycline-inducible promoter. The gRNA expression is repressed during mitotic growth and is activated at the onset of meiosis (T=0 h) when doxycycline is added in the sporulation medium. As a result, the frequencies of targeted DSBs and recombinant molecules detected during the mitotic growth decreased 6-(1.9 vs 12.0) and 20-fold (0.8 vs 17.0%), respectively. With this method, FnCpf1 is predominantly induced in meiosis and target DSBs, reaching a frequency of 17% at T=8h.

Controlling CRISPR SpCas Activity with a Doxycycline-Inducible Promoter and an Auxin-Inducible Degron System (FIG. 2)

In cells where the sgRNA expression is controlled by a doxycycline-inducible promoter the frequencies of mitotic SpCas9-induced DSBs and recombinant molecules are low (6.0 and 3.0%, respectively) (left panel). To eliminate the mitotic leak of SpCas9 activity, the auxin-inducible degron system was used to target SpCas9 degradation (right panel). In this context, auxin was added in the growth medium to induce the SpCas9 protein degradation. As a result, the frequencies of targeted DSBs and recombinant molecules detected during the mitotic growth were barely detectable (<0.3%). Then, upon addition of doxycycline at the onset of meiosis, SpCas9 was observed to efficiently induced targeted DSBs (up to 13%) and recombinant molecules (up to 2.1%) during meiosis progression.

Clustered Targeting of dCas9-Spo11 Enhances DSB Targeting Efficiency (FIG. 3)

To enhance the targeting efficiency of DNA Double-Strand Break (DSB) formation at a single locus, the inventors used poly-sgRNAs (up to 10) constructs allowing to target dCas9-Spo11 to sequences clustered in a small region (527 bp) of the GAL2 promoter in the same diploid cell. All the poly-sgRNAs constructs led to a higher frequency of targeted meiotic DSB formation (up to 18% with the 5 sgRNAs set) compared to individual sgRNAs.

### Meiotic SpCas9-Induced DSBs are Resected (FIG. 4).

In order to investigate whether meiotic SpCas9-induced DSBs are recombinogenic the inventors examined the pattern of SpCas9-DSBs in a dmc1Δ mutant when SpCAS9 is expressed under the meiosis-specific REC8 promoter and the sgRNA GAL2-D/E under the doxycycline-inducible RPR1 promoter (TetON system). In the absence of the meiosis-specific Dmc1 recombinase, the resected DSBs are not repaired and accumulate in the cells (Bishop, et al., 1992, Cell, 69, 439-456,), Comparison of the meiotic SpCas9-DSB band in the DMC1 and dmc1Δ strains revealed striking differences. SpCas9-DSB DNA from DMC1 cells formed a discrete band while SpCas9-DSB DNA from dmc1Δ cells appeared in a band with a smear due to 5′ to 3′ resection. This result shows that meiotic SpCas9-DSBs are resected and, consequently, seems to be prone to initiate meiotic recombination.

### SpCas9 Stimulates Meiotic Recombination at the Targeted GAL2 Region (FIG. 5).

As shown in FIG. 2, the targeting of SpCas9 to the GAL2 region led to a strong stimulation of meiotic DSB formation compared to WT cells (17 vs 0.6% at T=8 h), in a context where SpCAS9 is expressed under the meiosis-specific REC8 promoter and the sgRNA GAL2-_{D/E} is under the doxycycline-inducible RPR1 promoter (TetON system) (FIG. 5A). Importantly, the targeted SpCas9-DSBs led to a 2-fold stimulation of recombinant molecule frequency in comparison to the wild-type SPO11 strain (4.1 vs 1.8% at T=8 hours) (FIG. 5A). To confirm this result the inventors measured meiotic recombination in the progeny by dissecting a sample of four-spore tetrads and analyzing the segregation of the drug-resistance flanking markers. First, the inventors observed that spore viability is high in the SpCAS9 strain (84%) indicating that the majority of meiotic SpCas9-induced DSBs were repaired (FIG. 5B). The inventors found a significant 2.8-fold increase of crossovers in the SpCAS9 strain compared to the wild-type strain (5.9 vs 2.1 cM) (FIG. 5B). These genetic data are in agreements with the 2-fold increase of recombinant molecule frequency observed in FIG. 5A. Taken together, these results show that the targeting of SpCas9 stimulates meiotic recombination.

The inventors also examined the targeting of SpCas9 in SPO1l deleted cells. Like in SPO1l cells, SpCas9SpCas9 efficiently induced meiotic DSB formation in spo11Δ cells (15% at T=8). However, in contrast to SPO11 cells, the recombinant molecules are not detectable in SPO11 deleted cells.

This result shows that the endogenous Spo11 protein is required to initiate meiotic recombination with SpCas9-induced DSBs.

### Co-Expression of Three Distinct 2RNAs Allows Poly-Targeting of SpCas9 and FnCpf1 in the GAL2 Promoter (FIG. 6).

In order to direct SpCas9 and FnCpf1 in different sites within the GAL2 promoter, the gRNAs targeting the GAL2-A, -B and -D/E sites were co-expressed in the SpCAS9 and FnCPF1 cells. The poly-targeting of SpCas9 and FnCpf1 led to a high level of DSB frequency (40 and 42% at T=6 hours, respectively) distributed in discrete bands, whose migration corresponds to DSB formation at each target site. Thus, directing SpCas9 with 3 distinctive sgRNAs led to a 3.5-fold stimulation of targeted DSBs (42% at T=6 hours) compared to a single sgRNA (12% at T=6 hours) (FIG. 4). While SpCas9 induced DSB formation at the three target sites, FnCpf1 targeting was inefficient at the GAL2-D/E target site. Importantly, from T=6 hours SpCas9- and FnCpf1-DSB DNA formed a smeared band in the absence of the DMC1 recombinase, indicating that poly-targeted DSBs were resected.

### Poly-Targeting of SpCas9 and FnCpf1 Enhanced DSB Formation and Recombinant Molecules in the Targeted GAL2 Region (FIG. 7).

To verify that the poly-targeted DSBs enhanced meiotic recombination in the GAL2 region, the inventors examined the poly-targeting of SpCas9 and FnCpf1 in the recombination reporter strain by physical analyses of the recombinant molecules (FIG. 7A). As shown in FIG. 6, poly-targeting of the CRISPR endonucleases resulted in high targeted DSB frequency (22 and 21% for SpCas9 and FnCpf1, respectively) (FIG. 7B). Noteworthy, the DSB frequency dropped after 4 hours in the sporulation medium (22 vs 8% and 21 vs 8% for SpCAS9 and FnCPF1, respectively) due to the addition of auxin at T=5 hours, that promotes the degradation of the CRSIPR proteins. Importantly, these high frequencies of targeted DSBs enhanced meiotic recombination in the GAL2 promoter. Effectively, the inventors observed 10 and 15% of recombinant molecule frequency for SpCAS9 and FnCPF1, respectively, at T=8 hours 25 (FIGS. 7A and C). When compared to the targeting of SpCas9 with a unique sgRNA (FIG. 5A), the poly-targeting of SpCas9 with 3 different sgRNAs led to a 2.5-fold stimulation of recombinant molecule formation (10.0 vs 4.1 at T=8 hours). This represents a 5.5-increase of meiotic recombination in comparison to the wild-type control (10.0 vs 1.8) (FIGS. 5A and 7C).

### Comparison of SpCas9 Poly-Targeting and Co-Targeting of SpCas9 Plus SpdCas9-Spo11 in the GAL2 Region (FIG. 8).

To stimulate meiotic recombination in the GAL2 region, the inventors examined the poly-targeting of SpCas9 in the GAL2 gene sequence in the recombination reporter strain by physical analyses of the recombinant molecules (FIG. 8A). Poly-targeting of SpCas9 induced meiotic DSB formation in the GAL2 gene sequence (3.5% of total DSB frequency at T=4 hours) (FIG. 8B) with an efficiency that varies from site to site (FIG. 8A). Noteworthy, the DSB frequency dropped after 4 hours in the sporulation medium (3.5 vs 1.2%) due to the addition of auxin at T=5 hours, that promotes the degradation of the CRISPR protein. Importantly, these targeted DSBs enhanced meiotic recombination in the GAL2 region. Effectively, the inventors observed 6.9% of recombinant molecule frequency at T=8 hours (FIGS. 8A and C), which corresponds to a 3.5-fold increase in comparison to WT (6.9 vs 2.0%). To boost the efficiency of the SpCas9 targeting, the SpdCAS9-SPO11 gene fusion was co-expressed with the SpCAS9 gene and the 7 sgRNAs to target SpdCas9-Spo11 to the Cas9 target sites, and to promote the repair of DSBs by homologous recombination. When compared to the poly-targeting of sole SpCas9, the poly-targeting of SpCas9 and SpdCas9-Spo11 led to a 1.8-fold stimulation of DSB formation (6.2 vs 3.5% at T=4 hours) (FIG. 8B). Importantly, this results in a 1.4-fold increase in recombinant molecules (9.9 vs 6.9 at T=8 hours) (FIGS. 8A and C) and represents a 5.0-fold increase of in comparison to the wild-type control (9.9 vs 2.0).

### Poly-Targeting of SpCas9 Enhanced Meiotic Recombinant Molecules in the Targeted Pericentromeric GAL3 Region (FIG. 9).

The pericentromeric GAL3 region is a DSB-cold region where DSB formation is repressed (Robine, et al., 2007, Mol. Cell. Biol., 27, 1868-1880). As expected, no DSBs were detected in WT cells (FIG. 9A). Remarkably, the targeting of SpCas9 with the sgRNA-4 stimulated the frequency of DSB formation 20-fold in the GAL3 promoter (6.1 vs 0.3% at T=4 hours). However, this did not enhance the frequency of recombinant molecules compared to WT cells (1.1 vs 2.1% at T=8 hours) (FIGS. 9A and C). Noteworthy, the DSB frequency dropped after 4 hours in the sporulation medium (6.1 vs 2.2%) due to the addition of auxin at T=5 hours, that promotes the degradation of the CRSIPR proteins. As the poly-targeting of SpCas9 was shown to stimulate meiotic recombinant molecules in the targeted GAL2 promoter and coding sequence (FIGS. 7 and 8) the inventors co-expressed SpCas9 with 6 distinctive sgRNAs targeting the GAL3 promoter. The poly-targeting of SpCas9 led to a high level of DSB frequency (22% at T=4 hours) distributed in discrete bands, whose migration corresponds to DSB formation at 2 out the 6 target sites (#5 and 6) (FIG. 9A). Thus, directing SpCas9 with 6 distinctive sgRNAs led to a 3.6-fold stimulation of targeted DSBs (22% at T=4 hours) compared to the single sgRNA-4 (6.1% at T=4 hours) (FIG. 9B). Importantly, these high frequencies of targeted DSBs enhanced meiotic recombinant molecules in the GAL3 promoter. Effectively, the inventors observed 11% of recombinant molecule frequency at T=8 hours (FIGS. 9A and C). When compared to the targeting of SpCas9 with the unique sgRNA-4, the poly-targeting of SpCas9 with 6 different sgRNAs led to a 10-fold stimulation of meiotic recombinant molecules (11.0 vs 1.1 at T=8 hours). This represents a 5-fold increase in comparison to the wild-type control (11.0 vs 2.1) (FIGS. 9A and 9C).

### Targeting of Meiotic CRISPR-Mediated DNA Double-Strand Breaks in Tomato.

### a) Construction of the Cas9 and Cpf1 CRISPR Vector

In the present example, the CRISPR system is used to target meiotic DSBs in Tomato. To achieve the heterologous expression of the Cas9 protein (Class II) from Streptoccocus pyogenes or the Cpf1 protein (Class V) from Francisella novicida, the following components are assembled to generate a transgenic fusion construct expressing a “CRISPR-fused” protein in vivo. The first component is an Arabidopsis thaliana codon-optimized nucleotide sequence of the CAS9 gene and CPF1 genes. The second component is a nuclear localization signal (NLS, PKKKRKV SEQ ID NO: 106) fused to the N-terminus of the protein. The third component is a tag (3×FLAG,) attached to the C-terminus of the protein allowing the detection by immuno-precipitation of the CRISPR-Fused protein. Then, to achieve transcriptional expression, the CRISPR-Fused construct is expressed under an ubiquitous promoter (e.g.,: AtUbi), expressed in somatic and meiotic cells or under a meiosis-specific promoter, corresponding to the upstream 5′ region of a tomato gene induced in the prophase of meiosis, such as SPO11-1, DMC1 or REC8. Finally, a transcriptional terminator sequence (tNOS or pea3A) is added 3′ of the CRISPR-fused coding region.

For transformation, a binary vector (e.g., pGWB401) is used for stable transformation of young cotyledons, using T-DNA infection, as previously described (T. Reemet al., Plant Genome Ed. with Cris. Syst. Methods Mol. Biol. 1917 (2023)). The vector contains the Chloramphenicol/Spectinomycin and Kanamycin resistance genes for bacterial and transgenic plant selection, respectively. Plant transformants carrying the CRISPR-fused construct integrated in their genome are selected on growth medium supplemented with Kanamycin.

b) Construction of the CRISPR gRNA Component

The CRISPR nuclease targets specific DNA sequence via its cognate sequence-specific gRNA moiety (see M. Jinek, et al., Science (80). 337, 816-821 (2012)). These two components can be directly introduced by transformation in the same plant or brought together by crossing. Bioinformatic software (e.g., CRISPR PLANT) is used to identify the CRISPR target site(s) in the region(s) of interest. The target-specifying sequences (spacers) that are complementary to the target sequences (protospacers) are designed, synthesized and fused to the gRNA scaffold sequence that associates to the endonuclease. To simultaneously co-express multiple gRNAs, a synthetic polycistronic sequence carrying a cluster of gRNAs is designed, allowing the production of poly gRNAs from a primary transcript. For the CRISPR-Cas9 system, the polycistronic gene sequence consists of tandem repeats of tRNA-gRNA where the tRNA^{Gly} sequence is used to hijack the endogenous tRNA-processing system (see K. Xie, et al., Proc. Natl. Acad. Sci. U.S.A 112, 3570-3575 (2015) and Y. Zhang, et al., Nat. Commun. 12, 1-11 (2021)). For the CRISPR-Cpf1 system, the artificial pre-crRNA array is composed of spacers that are separated by direct repeats from the locus of F. novicida (B. Zetsche, et al., Cell 163, 759-771 (2015)). In the host plant, mature individual gRNAs (Cas9) and crRNAs (Cpf1) are released from the primary transcript upon excision by the endogenous RNAse P & RNAse Z, and Cpf1 itself, respectively. gRNA expression is driven by the constitutive A. thaliana RNA Pol III U6 promoter (see Z. Shimatani, et al. Nat. Biotechnol. 35, 441-443 (2017)).

### Single Binary Vector

The gRNA construct is integrated in the same vector as the CRISPR-Fused gene construct. The gene encoding the CRISPR endonuclease and the multiple gRNAs could have their own promoters or combined to generate a single long transcript under the control of the meiosis-specific promoter (Y. Zhang, et al., Nat. Commun. 12, 1-11 (2021)).

### Separated Binary Vectors

When, the gRNAs and CRISPR-fused constructs are cloned in distinct binary vectors the Geneticin resistance gene (ex: pCAMBIA2300) is used to allow a double selection for the integration of the T-DNA bearing the CRISPR-fused gene construct and the T-DNA bearing the cognate gRNA gene(s).

### c) Transformation Strategies

Co-transformation with two binary vectors carrying the genes encoding the CRISPR-fused endonuclease and the cognate gRNA(s). The vectors are introduced in the same or in two distinct bacteria strains that are mixed before the co-culture with the tomato cells.
Sequential transformation: stable transformants carrying the CRISPR-fused gene construct are generated to produce calli that will be used for the successive Agrobacterium- or biolistic-transformation with the gRNA construct.
Independent transformation: the transgenic plants carrying the CRISPR-fused gene construct and the single or multiplex gRNA constructs are generated separately and crossed to associate these two components of the CRISPR systems. Multiple transgenic plants expressing various sets of gRNAs are constructed and crossed to the CRISPR endonuclease-expressing transgenic plants in order to target different chromosomal regions.

### d) Tomato Transformation

By separate transformation of embryos from polymorphic parental lines, one line is transformed with the CRISPR-fused gene construct and the other by the gRNA construct. After validation of the transgene insertion upon PCR of genomic DNA extracted from leaves, the validated heterozygous transgenic plants are selected and crossed with lines heterozygous for the gRNA construct, allowing the formation of an active CRISPR RNA/protein complex.

By direct transformation of embryos from polymorphic F1 seeds: calli are induced from embryos obtained from mature polymorphic F1 seeds. Next, calli are simultaneously transformed with the T-DNA or the T-DNAs carrying the CRISPR-fused gene construct and the gRNA genes (co-transformation). Transformants expressing no gRNA or gRNA(s) targeting other genomic regions are used as control to monitor the targeting system efficiency.

### e) Recombination Assays

To detect meiotic crossover, the inventors use the heterozygous markers flanking the targeted site in the F1 plants. Frequencies of recombination in the targeted region(s) are measured by pollen typing of the polymorphic markers located on either side of the CRISPR targeted region or in the F2 plants. The stimulation of CRISPR-induced meiotic recombination in the interval is determined by comparing the frequency of recombinants in the control wild-type plant (without the CRISPR components) and the engineered CRISPR plants.

1-31. (canceled)

32. A method for inducing targeted meiotic recombination in a cell, said method comprising:
introducing into said cell:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
wherein the activity of the CRISPR nuclease is repressed outside of meiosis,
wherein the CRISPR nuclease is not fused with a Spo11 protein, and
wherein the cell is a yeast cell, a plant cell or a fungus cell.

33. A method for inducing targeted meiotic recombination in a cell, said method comprising:
introducing into said cell:
a) a CRISPR nuclease or a nucleic acid encoding said nuclease,
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region; and
c) a fusion protein comprising a Spo11 protein operably linked to a DNA binding domain, or a nucleic acid encoding said fusion protein, optionally under the control of a meiosis-specific promoter; and
inducing said cell to enter meiotic prophase I,
wherein the CRISPR nuclease is not fused with a Spo11 protein,
optionally wherein the activity of the CRISPR nuclease is repressed outside of meiosis, and
wherein the cell is a yeast cell, a plant cell or a fungus cell.

34. The method according to claim 33, wherein:
a) the activity of the CRISPR nuclease is repressed outside of meiosis;
b) the activity of the CRISPR nuclease is repressed outside of meiosis by placing the expression of said CRISPR nuclease and/or said guide RNAs under the control of a promoter inducible during the meiotic phase or under the control of a meiosis-specific promoter;
c) the activity of the CRISPR nuclease is repressed outside of meiosis by placing the expression of said CRISPR nuclease and/or said guide RNAs under the control of a promoter inducible during the meiotic phase or under the control of a meiosis-specific promoter and the meiosis specific promoter is selected from the group consisting of AtDMC1 promoter, Rec8 promoter, Spo13 promoter, MGE1p promoter, MGE2p promoter, MGE3p promoter, ZmDMC1 promoter, Maize Spo11-1 promoter (ZmSPO11-1), Tomato Spo11-1 promoter (SISPO1-1) and Arabidopsis Spo11-1 promoter (AtSPO11-1), and variant thereof exhibiting at least 80%, 85%, 90%, 95%, 96%, 97%, 98%, or at least 99% identity to any of these promoters and a meiosis specific promoter activity;
d) the activity of the CRISPR nuclease is repressed outside of meiosis by expressing an inhibitor of CRISPR endonuclease activity outside of meiosis;
e) the activity of the CRISPR nuclease is repressed outside of meiosis by placing the expression of the CRISPR nuclease and/or the guide RNA(s) under the control of a promoter inducible during the meiotic phase or a meiosis-specific promoter and by expressing an inhibitor of CRISPR endonuclease activity outside of meiosis;
f) the activity of the CRISPR nuclease is repressed outside of meiosis by using a system triggering the degradation of the CRISPR nuclease outside of meiosis;
g) the activity of the CRISPR nuclease is repressed outside of meiosis by placing the expression of the CRISPR nuclease and/or the guide RNA(s) under the control of a promoter inducible during the meiotic phase or a meiosis-specific promoter and by using a system triggering the degradation of the CRISPR nuclease and/or the guide RNA(s) outside of meiosis; or
h) the activity of the CRISPR nuclease is repressed outside of meiosis by using a CRISPR nuclease which is a variant of a wild-type CRISPR nuclease, said variant exhibiting a reduced endonuclease activity by comparison to said wild-type CRISPR nuclease.

35. The method according to claim 34, wherein the system triggering the degradation of the CRISPR nuclease outside of meiosis is an auxin-inducible degron system.

36. The method according to claim 33, wherein the cell comprises a plurality of guide RNAs targeting the same chromosomal region, or a plurality of nucleic acids encoding said guide RNAs, said chromosomal region being of less than 30 kilobase (kb) long.

37. The method according to claim 36, wherein the cell comprises at least five guide RNAs targeting the same chromosomal region or a plurality of nucleic acids encoding said guide RNAs, said chromosomal region being of less than 30 kilobase (kb) long.

38. The method according to claim 33, wherein the cell comprises seven or more guide RNAs targeting the same chromosomal region or a plurality of nucleic acids encoding said guide RNAs, said chromosomal region being of less than 30 kilobase (kb) long.

39. The method according to claim 33, wherein the cell comprises ten or more guide RNAs targeting the same chromosomal region or a plurality of nucleic acids encoding said guide RNAs, said chromosomal region being of less than 30 kilobase (kb) long.

40. The method according to claim 33, wherein the cell further comprises a fusion protein comprising a Spo11 protein operably linked to a DNA binding domain, or a nucleic acid encoding said fusion protein, optionally under the control of a meiosis-specific promoter.

41. The method according to claim 33, wherein the CRISPR nuclease is selected from the group consisting of Cas9 and Cpf1 nucleases or the CRISPR nuclease is a dead Cas9 (dCas9) or a dead Cpf1 (dCpf1).

42. The method according to claim 33, wherein the Spo11 protein is Spo11-1, Spo11-2 or Spo11-3.

43. The method according to claim 33, wherein the Spo11 protein is from Oryza sativa, Brassica campestris, Zea mays, Capsicum baccatum, Carica papaya or Solanum lycopersicum.

44. The method according to claim 33, wherein the cell does not comprise a fusion protein comprising a CRISPR nuclease and a Spoil partner, in particular a Spoil partner selected from the group consisting of Rec102, Rec103/Ski8, Rec104, Rec114, Mer1, Mer2/Rec107, Mei4, Mre2/Nam8, Mre11, Rad50, Xrs2/Nbs1, Hop1, Red1, Mek1, Set1 and Spp1, and variants and orthologues thereof, said variants having at least 80, 85, 90, 95, 96, 97, 98, or at least 99% sequence identity thereto.

45. The method according to claim 33, wherein the cell does not comprise a fusion protein comprising a CRISPR nuclease and a topoisomerase, optionally selected from the TOPOVIB or MTOPOVIB family or a variant or orthologue thereof, said variants having at least 80, 85, 90, 95, 96, 97, 98, or at least 99% sequence identity thereto.

46. The method according to claim 33, wherein the cell does not comprise a fusion protein comprising i) a CRISPR nuclease and ii) a DNA binding domain, or a protein/amino acid sequence comprising at least 50 amino acids.

47. A method for generating variants of an organism, said method comprising:
introducing into a cell of said organism:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the activity of the CRISPR nuclease is repressed outside of meiosis,
wherein the CRISPR nuclease is not fused with a Spo11 protein, and
wherein the organism is a yeast, a plant or a fungus.

48. A method for generating variants of an organism, said method comprising:
introducing into a cell of said organism:
a) a CRISPR nuclease or a nucleic acid encoding said nuclease, and
b) a plurality of guide RNAs targeting the same chromosomal region or a plurality of nucleic acids encoding said guide RNAs, said guide RNAs comprising an RNA structure for binding to the CRISPR nuclease and a sequence complementary to a targeted chromosomal region and
inducing said cell to enter meiotic prophase I,
obtaining a cell or cells having the desired recombination(s) at the targeted chromosomal region(s); and
generating a variant of the organism from said recombinant cell,
wherein the CRISPR nuclease is not fused with a Spo11 protein and wherein the activity of the CRISPR nuclease is repressed outside of meiosis, and
wherein the organism is a yeast, a plant or a fungus.

49. A host cell comprising:
a) a CRISPR nuclease or a nucleic acid encoding said nuclease, and
b) one or more guide RNAs or one or more nucleic acids encoding said guide RNAs, said guide RNAs comprising an RNA structure for binding to the CRISPR nuclease and a sequence complementary to a targeted chromosomal region, and
c) optionally a fusion protein comprising a Spo11 protein operably linked to a DNA binding domain, or a nucleic acid encoding said fusion protein,
wherein the CRISPR nuclease is not fused with a Spo11 protein and
wherein the activity of the CRISPR nuclease is repressed outside of meiosis by
(i) placing the expression of said CRISPR nuclease and/or said guide RNAs under the control of a promoter inducible during the meiotic phase or a meiosis-specific promoter, and/or
(ii) expressing an inhibitor of CRISPR endonuclease activity outside of meiosis, and/or
(iii) using a system triggering the degradation of the CRISPR nuclease outside of meiosis, and/or
(iv) using a CRISPR nuclease which is a variant of a wild-type CRISPR nuclease, said variant exhibiting a reduced endonuclease activity by comparison to said wild-type CRISPR nuclease;
wherein the host cell is a yeast cell, a plant cell or a fungus cell.

50. The host cell according to claim 49, wherein the host cell is a yeast cell or a plant cell.

51. The host cell according to claim 50, wherein the cell is a plant cell.

52. The host cell according to claim 51, wherein the cell is a plant cell selected from the group consisting of rice, wheat, soybean, corn, tomato, onion, cucumber, lettuce, asparagus, carrot, turnip, Arabidopsis thaliana, millet, barley, rapeseed, cotton, grapevine, sugar cane, beet, cotton, sunflower, oil palm, coffee, tea, cocoa, chicory, bell pepper, chili, lemon, orange, nectarine, mango, apple, banana, peach, apricot, sweet potato, yams, almond, hazelnut, strawberry, melon, watermelon, olive, potato, zucchini, eggplant, avocado, cabbage, plum, cherry, pineapple, spinach, apple, tangerine, mandarin, grapefruit, pear, grape, clove, cashew, coconut, sesame, rye, hemp, tobacco, berries, raspberry, blackcurrant, peanut, castor beans, vanilla, poplar, eucalyptus, green foxtail, cassava, roses, tulips, orchids, rubber tree and geraniums.

53. The host cell according to claim 52, wherein the cell is a tomato cell, a rice cell, a corn cell or a soja cell.

54. The host cell according to claim 53, wherein the cell is a tomato cell.


SEQUENCE LISTING (106 sequences)

SEQ ID NO: 1 - protein, 1368 aa, Streptococcus pyogenes [sequence omitted]

SEQ ID NO: 2 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 5 - protein, 1629 aa, Francisella tularensis
  source 1..1629: note = subsp. novicida
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 6 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 7 - protein, 1352 aa, unidentified
  source 1..1352: note = Parcubacteria
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 8 - protein, 1477 aa, unidentified
  source 1..1477: note = Candidatus Peregrinibacteria
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 9 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 11 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 12 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 13 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 14 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 15 - protein, 1263 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 16 - protein, 1229 aa, unidentified
  source 1..1229: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHK

SEQ ID NO: 17 - protein, 1300 aa, Francisella tularensis
  source 1..1300: note = subsp. novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 18 - protein, 1300 aa, synthetic construct
  REGION 1..1300: note = dead FnCpf1
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 19 - protein, 1229 aa, synthetic construct
  REGION 1..1229: note = dead LbCpf1
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHK

SEQ ID NO: 20 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 21 - protein, 1437 aa, unidentified
  source 1..1437: note = Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 22 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 23 - DNA, 1000 bp, Zea mays
  source 1..1000: mol_type = genomic DNA
tttatagcaaggcccgtttggttagagagactaattttagtccctgacttttagtctcatttagtctctattttgccaaacggaaggactaaagtagggactaattggttttagggcatgtttggttcgttacctcaattgccacattttgcctaacttttctgcctaaggttagttattcaattcgaataactaaccttaggcaaagtggggcacagttagccacaaaccaaacaagcccttagtctttagtcccttacatagatgctaaaagggactaaaggggaatatttactctaattacccttgcctagaaaactagtgtgaaacaaaaaaaagagtattttgatctttatgtattacatttaatgtatttaaaatctgtttagcccctacaactaaacaatatagagactaaagtttagtttagggactaaactttagtcctaagactaatggagccaaacggggcccaaatctcgtttaaaaatttgaagcaaacacatccttaacggaccgtgggcaatgagctgagtctcccctgactctgggccgcaaagtcctggtacacttgatcatgttggtccatcctatggctgaccgcgacttccctagactgaaatcaacccattcctaggctcctagcccagcccaacggccgagcacacgatccgttcgagagcgagaaggtcctccggcctcagcaccctcaagtacacagtacaccctcgcaccggtaccgcggtccgcggcacacgacacccccactcgtctgtcgactcacgtctgatcgtctctccccaacaatctctcgagtcttgccattcgcctcagcttgcccctcctccaagcgtccaagccccacccggccattgcctcctcctcctgccgcaggtaagctagctgctccagcttctcttgccatcgcgtgtcctgcactcaccgccctcgcgcgtgtaactcctcctccgtcccccgatcgggctactagtgcaggcac

SEQ ID NO: 24 - protein, 44 aa, synthetic construct
  REGION 1..44: note = AID-tag
PKDPAKPPAKAQVVGWPPVRSYRKNVMVSCQKSSGGPEAAAFVK

SEQ ID NO: 25 - protein, 398 aa, Saccharomyces cerevisiae
MALEGLRKKYKTRQELVKALTPKRRSIHLNSNGHSNGTPCSNADVLAHIKHFLSLAANSLEQHQQPISIVFQNKKKKGDTSSPDIHTTLDFPLNGPHLCTHQFKLKRCAILLNLLKVVMEKLPLGKNTTVRDIFYSNVELFQRQANVVQWLDVIRFNFKLSPRKSLNIIPAQKGLVYSPFPIDIYDNILTCENEPKMQKQTIFPGKPCLIPFFQDDAVIKLGTTSMCNIVIVEKEAVFTKLVNNYHKLSTNTMLITGKGFPDFLTRLFLKKLEQYCSKLISDCSIFTDADPYGISIALNYTHSNERNAYICTMANYKGIRITQVLAQNNEVHNKSIQLLSLNQRDYSLAKNLIASLTANSWDIATSPLKNVIIECQREIFFQKKAEMNEIDARIFEYK

SEQ ID NO: 26 - protein, 398 aa, Saccharomyces cerevisiae
MALEGLRKKYKTRQELVKALTPKRRSIHLNSNGHSNGTPCSNADVLAHIKHFLSLAANSLEQHQQPISIVFQNKKKKGDTSSPDIHTTLDFPLNGPHLCTHQFKLKRCAILLNLLKVVMEKLPLGKNTTVRDIFFSNVELFQRQANVVQWLDVIRFNFKLSPRKSLNIIPAQKGLVYSPFPIDIYDNILTCENEPKMQKQTIFPGKPCLIPFFQDDAVIKLGTTSMCNIVIVEKEAVFTKLVNNYHKLSTNTMLITGKGFPDFLTRLFLKKLEQYCSKLISDCSIFTDADPYGISIALNYTHSNERNAYICTMANYKGIRITQVLAQNNEVHNKSIQLLSLNQRDYSLAKNLIASLTANSWDIATSPLKNVIIECQREIFFQKKAEMNEIDARIFEYK

SEQ ID NO: 27 - protein, 362 aa, Arabidopsis thaliana
MEGKFAISESTNLLQRIKDFTQSVVVDLAEGRSPKISINQFRNYCMNPEADCLCSSDKPKGQEIFTLKKEPQTYRIDMLLRVLLIVQQLLQENRHASKRDIYYMHPSAFKAQSIVDRAIGDICILFQCSRYNLNVVSVGNGLVMGWLKFREAGRKFDCLNSLNTAYPVPVLVEEVEDIVSLAEYILVVEKETVFQRLANDMFCKTNRCIVITGRGYPDVSTRRFLRLLMEKLHLPVHCLVDCDPYGFEILATYRFGSMQMAYDIESLRAPDMKWLGAFPSDSEVYSVPKQCLLPLTEEDKKRTEAMLLRCYLKREMPQWRLELETMLKRGVKFEIEALSVHSLSFLSEVYIPSKIRREVSSP

SEQ ID NO: 28 - protein, 487 aa, Oryza sativa
MDDSTDDDSYHPRKHYAYDRQVSSSRWRTSREYIRGPGPETHTTESAQDGQDPPAGVYSYGYFSGSGNDPQVQGHFVPEIQKYNPYVIFKGEQLPVPIWELPEEKVQDFHDRYFIAKDKSRVEARKTLNRLLEGNINTIERGHGYKFNIPKYTDNMEFNEEVKVSLAKAGKTISRSFCNANQREVASRTGYTIDLIERTLGAGLNISKRTVLYTNKDLFGDQSKSDQAINDICALTNIRRGSLGIIAAEKGIVVGNIFLELTNGKSISCSIGVQIPHRLDQIKDVCVEIGSRNIEYILVVEKHTMLNYLLEMDYHTNNNCIILTGCGMPTLQTRDFLRFLKQRTGLPVFGLCDPDPEGISILATYARGSCNSAYDNFNISVPSICWVGLSSSDMIKLNLSETNYSRLSREDKTMLKNLWQDDLSDVWKRRIEEMISFDKKASFEAIHSLGFDYFATNLLPDMINKVREGYVQVQEKKEPQDTEASED

SEQ ID NO: 29 - protein, 381 aa, Oryza sativa
  source 1..381: note = subsp. japonica
MAGREKRRRVAALDGEERRRRQEEAATLLHRIRGLVRWVVAEVAAGRSPTVALHRYQNYCSSASAAAASPCACSYDVPVGTDVLSLLHRGSHASRLNVLLRVLLVVQQLLQQNKHCSKRDIYYMYPSIFQEQAVVDRAINDICVLFKCSRHNLNVVPVAKGLVMGWIRFLEGEKEVYCVTNVNAAFSIPVSIEAIKDVVSVADYILIVEKETVFQRLANDKFCERNRCIVITGRGYPDIPTRRFLRYLVEQLHLPVYCLVDADPYGFDILATYKFGSLQLAYDANFLRVPDIRWLGVFTSDFEDYRLPDCCLLHLSSEDRRKAEGILSRCYLHREAPQWRLELEAMLQKGVKFEIEALSACSISFLSEEYIPKKIKQGRHI

SEQ ID NO: 30 - protein, 381 aa, Oryza sativa; identical to SEQ ID NO: 29
  source 1..381: note = subsp. japonica

SEQ ID NO: 31 - protein, 442 aa, Oryza sativa
  source 1..442: note = subsp. indica
MSEKKRRGGAGAGAASGSASKKPRVSTAASYAESLRSKLRPDASILATLRSLASACSKSKPAGSSSSSSSASKALAAEDDPAASYIVVADQDSASVTSRINRLVLAAARSILSGRGFSFAVPSRAASNQVYLPDLDRIVLVRRESARPFANVATARKATITARVLSLVHAVLRRGIHVTKRDLFYTDVKLFGDQAQSDAVLDDVSCMLGCTRSSLHVVASEKGVVVGRLTFADDGDRIDCTRMGVGGKAIPPNIDRVSGIESDALFILLVEKDAAFMRLAEDRFYNRFPCIILTAKGQPDVATRLFLRRLKVELKLPVLALVDSDPYGLKILSVYMCGSKNMSYDSANLTTPDIKWLGVRPSDLDKYRVPEQCRLPMTDHDIKVGKELLEEDFVKQNEGWVKELETMLRTRQKAEIQALSSFGFQYLTEVYLPLKLQQQDWI

SEQ ID NO: 32 - protein, 385 aa, Oryza sativa
  source 1..385: note = subsp. japonica
MAEAGVAAASLFGADRRLCSADILPPAEVRARIEVAVLNFLAALTDPAAPAISALPLISRGAANRGLRRALLRDDVSSVYLSYASCKRSLTRANDAKAFVRVWKVMEMCYKILGEGKLVTLRELFYTLLSESPTYFTCQRHVNQTVQDVVSLLRCTRQSLGIMASSRGALIGRLVVQGPEEEHVDCSILGPSGHAITGDLNVLSKLIFSSDARYIIVVEKDAIFQRLAEDRIYSHLPCILITAKGYPDLATRFILHRLSQTYPNMPIFALVDWNPAGLAILCTYKYGSISMGLESYRYACNVKWLGLRGDDLQLIPQSAYQELKPRDLQIAKSLLSSKFLQDKHRAELTLMLETGKRAEIEALYSHGFDFLGKYVARKIVQGDYI

SEQ ID NO: 33 - protein, 362 aa, Brassica campestris
MEGNFQISETTNLLRKIKEFTRSIVEDLAEGKSPEISINRFRNYCNDPEADCFCSSDEPKGREILTLRKRSQTYRIDMLLRVLLIVQQLLQENRHGSKRDIYYMHPSAFKAQSVVDRAIADICILFQCSRYNLNVVSVGNGLVMGWLKFREAGRKFDCLSSLNTAFPVPVLVEEVEDIVSLAEYILVVEKETVFQRLANDMFCKTNRCIVVTGRGYPDVSTRRFLRLLMEKLQLPVHCLVDCDPYGFEILATYRFGSMQMAYDIESLRAPEMKWLGAFPSDSDIYGVPHQCLLPLTEEDKKRTEAMLLRCYLKREMPQWRLELETMLKRGVKFEIEALSVHSLSFLSEVYIPSKIRSEGSFH

SEQ ID NO: 34 - protein, 380 aa, Brassica campestris
MEEDASMKFFSDQNTSYADILPPREVRARIEVSVLNLLRVLNSPDPAISDLSLINRKRSNSCINQGILTDVSYIFLSTSFTKSSLTNAKTAKAFVRVWKVMEMCFQILLQEKRVTQRELFYKLLCDSPDLFSSQIEVNRSVQDVVALLRCSRFSLGIMASTRGLVAGRLYLQEPGKEPVDCSACGASGFPISGDLNLLDNTVMTSDARYIILVEKHAIFHRLVEDRVFNHIPCVFITAKGYPDIATRFFLHRMSITFPHLPILALVDWNPAGLAILCTFKFGSIGMGLEAYRYACNVKWIGLRGDDLNLIPEESLVPLKAKDSQIAKSLLSSKILQENYREELSLMIETGKRAEIEALYCHGYSYLGKYIATKIVQGKYI

SEQ ID NO: 35 - protein, 382 aa, Zea mays
MAEANVAAASLFGADGRLCSADILAPPEVRARIEVAVLNFLAALASPSSPAISVLPLISRSSANCSLRSGLLNDVSSVYLSYTFCKRSLTHNPKAFVRVWKVMEMCYKILGEGKLVQQRELFYKLLSDSPKYFSCQRHVNQAIQDVVSLLRCTRQSLGVMASSRGALIGRLVLHEPDGEQIDCSILGASGHAITGDLNLLSKLNLSSGSRYIIVVEKDAVFQRLAEDRLYNQLPCILITAKGYPDIATRFILHRLSQTFPNMPIFALVDWNPAGLAILCTYKYGSISMGLESYRYACNVKWLGVRGGDLHLIPEDAFQELKPRDLQIAKSLMSSKFLQESHRAELALMVERGKRADIEALYSHGFDFLGKYIARKIVQGDYI

SEQ ID NO: 36 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQHLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSHEYIPQKIKQGMHL

SEQ ID NO: 37 - protein, 383 aa, Zea mays
MAGRDKRRRAAPLEGDEQQLRRRLEEAALLLRRIKGLVRWIVEEVAAGRSPSIVLHRYRNYCSSADSASPSPCACSYDIPVGTDVLSLLHKDYHTSRLNVLLRVLFVVQQLLQQNKHCSKRDIYYMYPSIFVEVAVVDRAINDICILFKCSRHNLNVVPVVKGLVMGWIRFMEGEKKVYCITSVNAAFSIPVDIEAIKDVVSVAHYILVVEKETVFQRLANDKFCERNRCIVITGRGYPDIPTRRFLRYLVELLHLPAYCLVDSDPYGFDILATYKFGSLQLAHDANLLRVPDIRWLGVFTSDFEEYCLPDCCLLRLSPEDRRKAEGILARCYLHREAPEWRSELEAMLQKGVKFEIEALSANSISFLSDEYIPQKIKQGMHL

SEQ ID NO: 38 - protein, 382 aa, Zea mays
MAEANVAAASLFGADRRLCSADILAPPEVRGRIEVAVLNFLAALASPSSPAISVLPLISRSSANCSLRSGLLNDVSSVYLSYTFCKRSLTHNPKAFVRVWKVMEMCYKILGEGKLAQQRELFYKLLSDSPKYFSCQRHVNQAIQDVVSLLRCTRQSLGVMASSRGALIGRLVLHEPDGEQIDCSILGASGHAITGDLNLLSKLNLSSGSRYIIVVEKDAVFQRLAEDRLYNQLPCILITAKGYPDIATRFILHRLSQTFPNMPIFALVDWNPAGLAILCTYKYGSISMGLESYRYACNVKWLGVRGGDLHLIPEGAFQELKPRDLQIAKSLMSSKFLQESHRAELALMVDRGKRADIEALYSHGFDFLGKYIARKIVQGDYI

SEQ ID NO: 39 - protein, 218 aa, Capsicum baccatum
MGWLRYAESGRKIDCISNPNTAYPIPVHVEEVNDILSVAQYVLVVEKESVFQRLANDQFCKRNRCVVITGRGYPDVPTRRFLRLLIDKLHLPVYCLVDCDPYGFDILTTYKFGSLQMAYDATFLQVSEIQWLGVFVQDSDNYSIPQQCLLPLTVEDKRKVKAMLHRCYLQREVPKWRFELELLLYKGVKFEIEALSVHSLTFLSHEYLPSKINNGGYI

SEQ ID NO: 40 - protein, 209 aa, Capsicum baccatum
MRNASEGQEGFTRSIVEDLARGNAPLIYIDRFRNYCTGTSGNCSCSSGLPTGKEAISLKRECHVRRLDILLRVLLIVQQLLQENRHGSKRDIYYMHPTVFKEQSVVDRAINDICILLQCSRHNLNVLHVLFTVKDSKILHIHAFDMGNGSSQVSERPREPHKFLALGGEFCLIFLLHDLSFSIAWGVRCLLEMGAYLYTVHISLQECAS

SEQ ID NO: 41 - protein, 382 aa, Carica papaya
MEDLGRSSMRFFSDQRICYADILPPNEARARIEVAVLNFLRILNSRDPAISSLPLVNRKWSNSRVSKGLLTDVSRIFLCRSFCIRSLMRVKATKAFVRVWKVMEMCFRILIQEKRVTQRELFYLLICDSPEYFSSQLEVNRTIQDVVALLQCSRYSLGIMASSRGIVAGRLLLQEPNQEVVDCSACGSSGYTISGDLNLIQTLILKTDARYIIVVEKHAIFQRLLEDGISNHIPSIIITAKGYPDIATRFLLHRMSSKFPGMPILALVDWNPAGLAILCTFKYGSIGMGLEAYRYACNVKWVGLRRDDLHLVPEQALIPLKPRDLQIAKSLMSSEVLQDEYREELASMIESGQRAEIEALFFHGYDYLGKYIAKKVVQANYI

SEQ ID NO: 42 - protein, 382 aa, Solanum lycopersicum
MEGKRSNTRPLTLLRKIKGFTRSIVEDLARGRPPLIYIERFRNYCTDTSGNCSCSSGLSTGKEAISLKRECHARRLDVMLRVLLIVQQLLQENRHGSKRDIYYMHPTVFKEQSVVDRAINDICILLQCSRHNLNVVSVGNGLVMGWLRYVESGRKIDCISNPNTAYPIPVLVEEVDDIISVAQYVIVVEKESVFQRLANDQFCKRNRCIVITGRGYPDVPTRRFLRLLIDKLHLPVYGLVDCDPYGFDILTTYKFGSLQMAYDATFLRVSEIQWLGVFVQDCDNYSIPQQCLLPLTAEDKRKIEAMLPRCYLQREVPQWRFELELLLHKGVKFEIEALSVHSLTFLSHEYLPSKIHNGGLKILSLNQMILHQNRLKTPISLN

SEQ ID NO: 43 - DNA, 25 bp, synthetic construct
  misc_feature 1..25: note = pGAL2 crRNA-B
  source 1..25: mol_type = other DNA
gtccgtgcggagatatctgcgccgt

SEQ ID NO: 44 - DNA, 20 bp, synthetic construct
  misc_feature 1..20: note = pGAL2 gRNA-A
  source 1..20: mol_type = other DNA
caattcggaaagcttccttc

SEQ ID NO: 45 - DNA, 20 bp, synthetic construct
  misc_feature 1..20: note = pGAL2 gRNA-B
  source 1..20: mol_type = other DNA
ttgcctcaggaaggcaccgg

SEQ ID NO: 46 - DNA, 20 bp, synthetic construct
  misc_feature 1..20: note = pGAL2 gRNA-D/E
  source 1..20: mol_type = other DNA
gatcactccgaaccgagatt

SEQ ID NO: 47 - DNA, 20 bp, synthetic construct
  misc_feature 1..20: note = pGAL2 gRNA-4
  source 1..20: mol_type = other DNA
atctcaagatggggagcaaa

SEQ ID NO: 48 - DNA, 20 bp, synthetic construct
  misc_feature 1..20: note = pGAL2 gRNA-5
  source 1..20: mol_type = other DNA
tcttaaattatacaacgttc

SEQ ID NO: 49 - DNA, 20 bp, synthetic construct
  misc_feature 1..20: note = pGAL2 gRNA-6
  source 1..20: mol_type = other DNA
acatttcgcaggctaaaatg

SEQ ID NO: 50 - DNA, 20 bp, synthetic construct
  misc_feature 1..20: note = pGAL2 gRNA-7
  source 1..20: mol_type = other DNA
cagtaattggattgaaaatt

SEQ ID NO: 51 - DNA, 20 bp, synthetic construct
  misc_feature 1..20: note = pGAL2 gRNA-8
  source 1..20: mol_type = other DNA
gttcaggggtccatgtgcct

SEQ ID NO: 52 - DNA, 20 bp, synthetic construct
  misc_feature 1..20: note = pGAL2 gRNA-9
  source 1..20: mol_type = other DNA
tttctattagtagctaaaaa

SEQ ID NO: 53 - DNA, 20 bp, synthetic construct
  misc_feature 1..20: note = pGAL2 gRNA-10
  source 1..20: mol_type = other DNA
caaggaggtttacggaccag

SEQ ID NO: 54 - DNA, 25 bp, synthetic construct
  misc_feature 1..25: note = pGAL2 crRNA-A
  source 1..25: mol_type = other DNA
agaaaaggtattcaacgtcaattcg

SEQ ID NO: 55 - DNA, 25 bp, synthetic construct
  misc_feature 1..25: note = pGAL2 crRNA-D/E
  source 1..25: mol_type = other DNA
ctccccatcttgagatgggaagggc

SEQ ID NO: 56 - DNA, 20 bp, synthetic construct
  misc_feature 1..20: note = GAL2-ORF sgRNA-1
  source 1..20: mol_type = other DNA
aagtggtaccaatatttcat

SEQ ID NO: 57 - DNA, 20 bp, synthetic construct
  misc_feature 1..20: note = GAL2-ORF sgRNA-2
  source 1..20: mol_type = other DNA
ttgggctactgtactaatta

SEQ ID NO: 58 - DNA, 20 bp, synthetic construct
  misc_feature 1..20: note = GAL2-ORF sgRNA-3
  source 1..20: mol_type = other DNA
atccccacgttatttatgtg

SEQ ID NO: 59 - DNA, 20 bp, synthetic construct
  misc_feature 1..20: note = GAL2-ORF sgRNA-4
  source 1..20: mol_type = other DNA
tagtgcacttaccccacgtt

SEQ ID NO: 60 - DNA, 20 bp, synthetic construct
  misc_feature 1..20: note = GAL2-ORF sgRNA-5
  source 1..20: mol_type = other DNA
gcgtcgtaaatgtttacttt

SEQ ID NO: 61 - DNA, 20 bp, synthetic construct
  misc_feature 1..20: note = GAL2-ORF sgRNA-6
  source 1..20: mol_type = other DNA
ttttctgttatgccacaacc

SEQ ID NO: 62 - DNA, 20 bp, synthetic construct
  misc_feature 1..20: note = GAL2-ORF sgRNA-7
  source 1..20: mol_type = other DNA
ataaccgtagtagaagttaa

SEQ ID NO: 63 - DNA, 20 bp, synthetic construct
  misc_feature 1..20: note = pGAL3 sgRNA-1
  source 1..20: mol_type = other DNA
aaagacaatgccaaatcatt

SEQ ID NO: 64 - DNA, 20 bp, synthetic construct
  misc_feature 1..20: note = pGAL3 sgRNA-2
  source 1..20: mol_type = other DNA
gattcttgctagccttttct

SEQ ID NO: 65 - DNA, 20 bp, synthetic construct
  misc_feature 1..20: note = pGAL3 sgRNA-3
  source 1..20: mol_type = other DNA
gtagaagataatagtaaaag

SEQ ID NO: 66 - DNA, 20 bp, synthetic construct
  misc_feature 1..20: note = pGAL3 sgRNA-4
  source 1..20: mol_type = other DNA
tagtgcacttaccccacgtt

SEQ ID NO: 67 - DNA, 20 bp, synthetic construct
  misc_feature 1..20: note = pGAL3 sgRNA-5
  source 1..20: mol_type = other DNA
attgaccgcctgaaacacat

SEQ ID NO: 68 - DNA, 20 bp, synthetic construct
  misc_feature 1..20: note = pGAL3 sgRNA-6
  source 1..20: mol_type = other DNA
gcactcctgattccgctaat

SEQ ID NO: 69 - DNA, 20 bp, synthetic construct
  misc_feature 1..20: note = pGAL3 sgRNA-7
  source 1..20: mol_type = other DNA
cgataaaatcaggtttgaca

SEQ ID NO: 70 - DNA, 25 bp, synthetic construct
  misc_feature 1..25: note = pGAL2 crRNA-A target sequence
  source 1..25: mol_type = other DNA
cgaattgacgttgaataccttttct

SEQ ID NO: 71 - DNA, 25 bp, synthetic construct
  misc_feature 1..25: note = pGAL2 crRNA-B target sequence
  source 1..25: mol_type = other DNA
acggcgcagatatctccgcacggac

SEQ ID NO: 72 - DNA, 25 bp, synthetic construct
  misc_feature 1..25: note = pGAL2 crRNA-D/E target sequence
  source 1..25: mol_type = other DNA
gcccttcccatctcaagatggggag

SEQ ID NO: 73 - DNA, 20 bp, synthetic construct
  misc_feature 1..20: note = pGAL2 gRNA-A target sequence
  source 1..20: mol_type = other DNA
gaaggaagctttccgaattg

SEQ ID NO: 74 - DNA, 20 bp, synthetic construct
  misc_feature 1..20: note = pGAL2 gRNA-B target sequence
  source 1..20: mol_type = other DNA
ccggtgccttcctgaggcaa

SEQ ID NO: 75 - DNA, 20 bp, synthetic construct
  misc_feature 1..20: note = pGAL2 gRNA-D/E target sequence
  source 1..20: mol_type = other DNA
aatctcggttcggagtgatc

SEQ ID NO: 76 - DNA, 20 bp, synthetic construct
  misc_feature 1..20: note = pGAL2 gRNA-4 target sequence
  source 1..20: mol_type = other DNA
tttgctccccatcttgagat

SEQ ID NO: 77 - DNA, 20 bp, synthetic construct
  misc_feature 1..20: note = pGAL2 gRNA-5 target sequence
  source 1..20: mol_type = other DNA
gaacgttgtataatttaaga

SEQ ID NO: 78 - DNA, 20 bp, synthetic construct
  misc_feature 1..20: note = pGAL2 gRNA-6 target sequence
  source 1..20: mol_type = other DNA
cattttagcctgcgaaatgt

SEQ ID NO: 79 - DNA, 20 bp, synthetic construct
  misc_feature 1..20: note = pGAL2 gRNA-7 target sequence
  source 1..20: mol_type = other DNA
aattttcaatccaattactg

SEQ ID NO: 80 - DNA, 20 bp, synthetic construct
  misc_feature 1..20: note = pGAL2 gRNA-8 target sequence
  source 1..20: mol_type = other DNA
aggcacatggacccctgaac

SEQ ID NO: 81 - DNA, 20 bp, synthetic construct
  misc_feature 1..20: note = pGAL2 gRNA-9 target sequence
  source 1..20: mol_type = other DNA
tttttagctactaatagaaa

SEQ ID NO: 82 - DNA, 20 bp, synthetic construct
  misc_feature 1..20: note = pGAL2 gRNA-10 target sequence
  source 1..20: mol_type = other DNA
ctggtccgtaaacctccttg

SEQ ID NO: 83 - DNA, 20 bp, synthetic construct
  misc_feature 1..20: note = GAL2-ORF sgRNA-1 target sequence
  source 1..20: mol_type = other DNA
atgaaatattggtaccactt

SEQ ID NO: 84 - DNA, 20 bp, synthetic construct
  misc_feature 1..20: note = GAL2-ORF sgRNA-2 target sequence
  source 1..20: mol_type = other DNA
taattagtacagtagcccaa

SEQ ID NO: 85 - DNA, 20 bp, synthetic construct
  misc_feature 1..20: note = GAL2-ORF sgRNA-3 target sequence
  source 1..20: mol_type = other DNA
cacataaataacgtggggat

SEQ ID NO: 86 - DNA, 20 bp, synthetic construct
  misc_feature 1..20: note = GAL2-ORF sgRNA-4 target sequence
  source 1..20: mol_type = other DNA
aacgtggggtaagtgcacta

SEQ ID NO: 87 - DNA, 20 bp, synthetic construct
  misc_feature 1..20: note = GAL2-ORF sgRNA-5 target sequence
  source 1..20: mol_type = other DNA
aaagtaaacatttacgacgc

SEQ ID NO: 88 - DNA, 20 bp, synthetic construct
  misc_feature 1..20: note = GAL2-ORF sgRNA-6 target sequence
  source 1..20: mol_type = other DNA
ggttgtggcataacagaaaa

SEQ ID NO: 89 - DNA, 20 bp, synthetic construct
  misc_feature 1..20: note = GAL2-ORF sgRNA-7 target sequence
  source 1..20: mol_type = other DNA
ttaacttctactacggttat

SEQ ID NO: 90 - DNA, 20 bp, synthetic construct
  misc_feature 1..20: note = pGAL3 sgRNA-1 target sequence
  source 1..20: mol_type = other DNA
aatgatttggcattgtcttt

SEQ ID NO: 91 - DNA, 20 bp, synthetic construct
  misc_feature 1..20: note = pGAL3 sgRNA-2 target sequence
  source 1..20: mol_type = other DNA
agaaaaggctagcaagaatc

SEQ ID NO: 92 - DNA, 20 bp, synthetic construct
  misc_feature 1..20: note = pGAL3 sgRNA-3 target sequence
  source 1..20: mol_type = other DNA
cttttactattatcttctac

SEQ ID NO: 93 - DNA, 20 bp, synthetic construct
  misc_feature 1..20: note = pGAL3 sgRNA-4 target sequence
  source 1..20: mol_type = other DNA
aacgtggggtaagtgcacta

SEQ ID NO: 94 - DNA, 20 bp, synthetic construct
  misc_feature 1..20: note = pGAL3 sgRNA-5 target sequence
  source 1..20: mol_type = other DNA
atgtgtttcaggcggtcaat

SEQ ID NO: 95 - DNA, 20 bp, synthetic construct
  misc_feature 1..20: note = pGAL3 sgRNA-6 target sequence
  source 1..20: mol_type = other DNA
attagcggaatcaggagtgc

SEQ ID NO: 96 - DNA, 20 bp, synthetic construct
  misc_feature 1..20: note = pGAL3 sgRNA-7 target sequence
  source 1..20: mol_type = other DNA
tgtcaaacctgattttatcg

SEQ ID NO: 97 - DNA, 816 bp, Zea mays
  source 1..816: mol_type = genomic DNA
aatctagaggtctcaggacgaattccatctaagtatcttggtaaagcatggattaatttggatgcccacttcaggtctatgcagctccggtgccttgtgattgtgagttgtgaccgatgctcatgctattctgcatttctgcgatgtatgtagctagtagatcttcaaaactaacaccgcatgccatcatcatccactgcttgattttagtctcaccgctggccaaaaatgtgatgatgccagaaacctcaactaccttgaatcaacacgggcccaacagtgtgatgacgacagaaacaaaaaaaaatgagccaatagttcagaaggaggcactatgcagaaactacatttctgaaggtgactaaaaggtgagcgtagagtgtaattactagtagtttagccaccattacccaaatgctttcgagcttgtattaagatttcctaagctgagcatcatcactgatctgcaggccaccctcgcttcgctgccaagatcaacagcaaccatgtggcggcaacatccagcattgcacatgggctaaagattgagctttgtgcctcgtctagggatcagctgaggttatcagtctttcctttttttcatccaggtgaggcatcaagctactactgcctcgattggctggacccgaagcccacatgtaggataccagaatgggccgacccaggacgcagtatgttggccagtcccaccggttagtgccatctcggttgctcacatgcgtagaagccagcttaaaaatttagctttggtgactcacagcgtggcagagacgagatctagtctgaggtcgactt

SEQ ID NO: 98 - DNA, 972 bp, Petroselinum crispum
  source 1..972: mol_type = genomic DNA
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagc

SEQ ID NO: 99 - DNA, 758 bp, synthetic construct
  misc_feature 1..758: note = 2x 35S Promoter
  source 1..758: mol_type = other DNA
gccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgasadhttrcgtaagggatgacgcacaatcccactatccttcgcaagacccttcctctatataaggaagttcatttcatttggagaggccgg

SEQ ID NO: 100 - DNA, 443 bp, Arabidopsis thaliana
  source 1..443: mol_type = genomic DNA
gttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattga

SEQ ID NO: 101 - DNA, 381 bp, Oryza sativa
  source 1..381: mol_type = genomic DNA
aaggaatctttaaacatacgaacagatcacttaaagttcttctgaagcaacttaaagttatcaggcatgcatggatcttggaggaatcagatgtgcagtcagggaccatagcacaagacaggcgtcttctactggtgctaccagcaaatgctggaagccgggaacactgggtacgttggaaaccacgtgatgtgaagaagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctgatttaaaagagttgtgcagatgatccgtggca

SEQ ID NO: 102 - DNA, 447 bp, Oryza sativa
  source 1..447: mol_type = genomic DNA
ttttttcctgtagttttcccacaaccattttttaccatccgaatgataggataggaaaaatatccaagtgaacagtattcctataaaattcccgtaaaaagcctgcaatccgaatgagccctgaagtctgaactagccggtcacctgtacaggctatcgagatgccatacaagagacggtagtaggaactaggaagacgatggttgattcgtcaggcgaaatcgtcgtcctgcagtcgcatctatgggcctggacggaataggggaaaaagttggccggataggagggaaaggcccaggtgcttacgtgcgaggtaggcctgggctctcagcacttcgattcgttggcaccggggtaggatgcaatagagagcaacgtttagtaccacctcgcttagctagagcaaactggactgccttatatgcgcgggtgctggcttggctgccg

SEQ ID NO: 103 - DNA, 1269 bp, Zea mays
  source 1..1269: mol_type = genomic DNA
agcttctcatcttggtctttcgatagctgttcttgaactgaaaggatgatttgacaaataagtgaactactaaattgtacatctagtttctattaactcagcacaactccaacatttgtctcttgtaaataactttcgcagtgctcctaattgtgttagttataatccacgaaaaggattgtatgttgacaaggaacagataagacctaatgctagcaaattcacttctgaacatcttaactcctaaaaaaaattcgaccttctcacgtaggtcaagaaaaccctgaacccctgtcacatccatacaaagtgatattatagcccatgtgagaacgaccgggaccggtccttagacctctgcttactcagcttaaaattcgctcccacgaggagtcgaatctatcctggtcactgcaccagccaagatgcagtgatacatgtcacttcaggcatgtagttacactttgcagtttgaaacaatggaaatgatccctgtgtaaaattggcgctatccctgccattctatatacactgcaatgtggcgaatgcgagtattggtggtgatgggagctgtgtaactgattgactcaatgtgcaggattgtccatccagctccaggcgttccagaggccctgatacaggagatgttccagcacaacccaggggtgtccagggagggtctcggcctgtacataagccagaagctggtgaaaacgatgagcggcacgttgcagtacctacgagaagccgacacctcttcgttcatcatcctgatagagttcccggtcgcccagctcagcagcaagcggtccaagccttcgccaagtaaattctgacactgatgctcctggaactgcagtgacggttcggccgtgtcaaattctcgggtgtagggtgtacctctggccaagtgactgatattctgaaaaacatacggacttctagtataggagaagaggaatcctaatcctagtaattatatcttagtagatatgtgcttagtctcttgttaattttttgtcttgagaggactatatatactgataaagtgataatttattcagagaataaagaatgggctccatgctttgtctatgtattctcttcttgagggcaatttacgcctcttgagaagggtggcttcgttgttgggctcttgaaggcccacgcagtccaacggagcccactaagcctaattctgtttcttcgcgatcgattagctgattagctgcatggcggcttcgaaaaaatcaaaataaaaaactagcg

SEQ ID NO: 104 - DNA, 1162 bp, Solanum lycopersicum
  source 1..1162: mol_type = genomic DNA
atatgagttttttgtcaaaaccatccctaaactataccacaaatttagattacaaccttcaaatatttaaaagcgaacagtttttatcttctgttagatatcgtcaaaaaattaagggatcgtacaaaaacatgagcagttcatgtgaccatcaataaagatttcctgcataatttcgttacttgctataaaaagttcttgaaaaaaacatattgaaatgtgtgaggatacttgatctgctctttttcttatttagtaacaaaaagaaggtggagcttaattatcttacctttttcatagtcaaactgtatttagatcaataatttttttggccaatctgacgttgaagtgatcaaaatattgattttgtcttgtatttgagtttcattctacattattcaaagtgaaagtctccaacaaatgttgttctagaatattcaatctcgagccgaaacctaggtcaagcagtggtttgagactcaatctcaatcatgatttcaaatcgagatcggaaatccaacctcgagccgagattattggaccttaatctcaagtcaaggtcgagtaccaaatcgaggtcaagtcaagtcttgaattgaaattggggttgagtctcgaattcaagactaattcaaaaattaaaacagagggggcatcatgacttaaaaagaaataaaaaacctatgcaatataacgccataaatttaatcaatgcatccagtaagttatgattaatacaccctcaagcaactaggtaggtccgcgctacgtctcgaatcaaagtcagtgttggaaatcatgaagttagatcccaactcaaggttggaaatagggcttgaggttgaggtcaagctaaagatcaagatcaagagttaggtattgagtgagggttgagatcaaaagttggttccttgaatggatagggttggtggtgaggttcgaattttttttcttaattttaagtggaatagttattatatctcatattgaaaaaatatttttcaaaatattttaagttaataaaatataaaaatttctaaaatatttttttttctaaaatattaaccttcatgcccaacacgccgagctaacttgcgcgcgcagctataaagctcggctttaccaatttacaaacaacgtgaacctgcagagcgaagaggaa

SEQ ID NO: 105 - DNA, 933 bp, Arabidopsis thaliana
  source 1..933: mol_type = genomic DNA
atattacttattttaatcttatgtttctgtttaggcgcgaggcttcttttctttaggtatctctcattagtttatacgttacaagattacacattgcattgatatatgattttactttcaaaaaaaatttaaatgttgaacgaccttaaaatccgtgcgctttacttttaaatctttccgaatgaaacccaaccaccgacgcgacagcttttcatttgtttactaattaaacggaacaaagcaatttaaatgttatcagtgttgtgttttagttagaaatactataaataatacaagatgcctcttttgttttagaacttttttggtatcatgaattcgttatcggataaaatacgttaaagctaagtagtatattcttagattatatataaattaggtgagcaatctatcatagagaagaaatgattaaaataagtgtatggcataatagattcgaacattttggtatgttcctcacgtgtccgagcataaagcttatgaacaaatctcgaccactagaagtctagaactaattctaatctagtttaccatacacattaattatttcaaatttagtgcacatgattattttatattggcatcactaaaatgtctccaagtctgaataaatcgatttatattggtttgattgttatcatgaatctttttcacaatcactttgcctagtttcggatcgggcctaaaagccaacggcccaacgtgtttaaaaacacatgcgtttttaaaatgacacgtcagttacgtatcgggcctaaatgccaatggcccaacgtcatggaaagtaggaactaaacgacacgcatgtttaaattcgaaggttttaacggcaagcttttcgcacccatcccttttcttcccgtctacaaaaattcgtcgaccattgcatttttcagttttgaaactcgaaagaga

SEQ ID NO: 106 - protein, 7 aa, synthetic construct
PKKKRKV